SEQUENCE LISTING (10 sequences)

SEQ ID NO: 1 - DNA, 7095 bp, Artificial Sequence
  Homo sapiens spectrin beta, non-erythrocytic 1 (SPTBN1), transcript variant 1
atgacgaccacagtagccacagactatgacaacattgagatccagcagcagtacagtgatgtcaacaaccgctgggatgtcgacgactgggacaatgagaacagctctgcgcggctttttgagcggtcccgcatcaaggctctggcagatgagcgtgaagccgtgcagaagaagaccttcaccaagtgggtcaattcccaccttgcccgtgtgtcctgccggatcacagacctgtacactgaccttcgagatggacggatgctcatcaagctgctggaggtcctctctggagagaggctgcctaaacccaccaagggacgaatgcgcatccactgcttagagaatgtggacaaggcccttcagttcctgaaggagcagagagtccatcttgagaacatggggtcccatgacatcgtggatggaaaccaccggctgacccttggcctcatctggaccatcatcctgcgcttccagatccaggatatcagtgtggaaactgaagacaacaaagagaagaaatctgccaaggatgcattgctgttgtggtgccagatgaagacagctgggtaccccaatgtcaacattcacaatttcaccactagctggagggacggcatggccttcaatgcactgatacacaaacaccggcctgacctgatagattttgacaaactaaagaaatctaacgcacactacaacctgcagaatgcatttaatctggcagaacagcacctcggcctcactaaactgttggaccccgaagacatcagcgtggaccatcctgatgagaagtccataatcacttatgtggtgacttattaccactacttctctaagatgaaggccttagctgttgaaggaaaacgaattggaaaggtgcttgacaatgctattgaaacagaaaaaatgattgaaaagtatgaatcacttgcctctgaccttctggaatggattgaacaaaccatcatcattctgaacaatcgcaaatttgccaattcactggtcggggttcaacagcagcttcaggcattcaacacttaccgcactgtggagaaaccacccaaatttactgagaaggggaacttggaagtgctgctcttcaccattcagagcaagatgagggccaacaaccagaaggtctacatgccccgggaggggaagctcatctctgacatcaacaaggcctgggaaagactggaaaaagcggaacacgaaagagaactggctttgcggaatgagctcataagacaggagaaactggaacagctcgcccgcagatttgatcgcaaggcagctatgagggagacttggctgagcgaaaaccagcgtctggtgtctcaggacaactttgggtttgaccttcctgcagttgaggccgccacaaaaaagcacgaggccattgagacagacattgccgcatacgaggagcgtgtgcaggctgtggtagccgtggccagggagctcgaggccgagaattaccacgacatcaagcgcatcacagcgaggaaggacaatgtcatccggctctgggaatacctactggaactgctcagggcccggagacagcggctcgagatgaacctggggctgcagaagatattccaggaaatgctctacattatggactggatggatgaaatgaaggtgctagtattgtctcaagactatggcaaacacttacttggtgtggaagacctgttacagaagcacaccctggttgaagcagacattggcatccaggcagagcgggtgagaggtgtcaatgcctccgcccagaagttcgcaacagacggggaaggttacaagccctgtgacccccaggtgatccgagaccgcgtggcccacatggagttctgttatcaagagctttgccagctggcggctgagcgcagggcccgtctggaagagtcccgccgcctctggaagttcttctgggagatggcagaagaggaaggctggatacgggagaaggagaagatcctgtcctcggacgattacgggaaagacctgaccagcgtcatgcgcctgctcagcaagcaccgggcgttcgaggacgagatgagcggccgcagtggccactttgagcaggccatcaaggaaggcgaagacatgatcgcggaggagcacttcgggtcggagaagatccgtgagaggatcatttacatccgggagcagtgggccaacctagagcagctctcggccattcggaagaagcgcctggaggaggcctccctgctgcaccagttccaggcagatgctgatgacattgatgcctggatgctggacatcctcaagattgtctccagcagcgacgtgggccacgatgagtattccacacagtctctggtcaagaaacacaaggacgtggcggaagagatcgccaattacaggcccacccttgacacgctgcacgaacaagccagcgccctcccccaggagcatgccgagtctccagacgtgaggggcaggctgtcgggcatcgaggagcggtataaggaggtggcagagctgacgcggctgcggaagcaggcactccaggacactctggccctgtacaagatgttcagcgaggctgatgcctgtgagctctggatcgacgagaaggagcagtggctcaacaacatgcagatcccagagaagctggaggatctggaggtcatccagcacagatttgagagcctagaaccagaaatgaacaaccaggcttcccgggttgcagtggtgaaccagattgcacgccagctgatgcacagcggccacccaagtgagaaggaaatcaaagcccagcaggacaaactcaacacaaggtggagccagttcagagaactggttgacaggaagaaggatgccctcctgtctgccctgagcatccagaactaccacctcgagtgcaatgaaaccaaatcctggattcgggaaaagaccaaggtcatcgagtccacccaggacctgggcaatgacctggctggcgtcatggccctgcagcgcaagctgaccggcatggagcgggacttggtggccattgaggcaaagctgagtgacctgcagaaggaggcggagaagctggagtccgagcaccccgaccaggcccaggccatcctgtctcggctggccgagatcagcgacgtgtgggaggagatgaagaccaccctgaaaaaccgagaggcctccctgggagaggccagcaagctgcagcagttcctacgggacttggacgacttccagtcctggctctctaggacccagacagcgatcgcctcggaggacatgccaaacaccctgaccgaggctgagaagctgctcacgcagcacgagaacatcaagaacgagatcgacaactacgaggaggactaccagaagatgagggacatgggcgagatggtcacccaggggcagaccgatgcccagtacatgtttctgcggcagcggctgcaggccctggacactggatggaacgagctccacaagatgtgggagaacagacaaaatctcctatcccagtcacatgcctaccagcagttcctcagagacacgaagcaagccgaagcctttcttaacaaccaggagtatgttctggctcacactgaaatgcctaccaccttggaaggagctgaagcagcaattaaaaagcaagaggacttcatgaccaccatggacgccaatgaggagaagatcaatgctgtggtggagactggccggaggctggtgagcgatgggaacatcaactcagatcgcatccaggagaaggtggactctattgatgacagacataggaagaatcgtgagacagccagtgaacttttgatgaggttgaaggacaacagggatctacagaaattcctgcaagattgtcaagagctgtctctctggatcaatgagaagatgctcacagcccaggacatgtcttacgatgaagccagaaatctgcacagtaaatggttgaagcatcaagcatttatggcagaacttgcatccaacaaagaatggcttgacaaaatcgagaaggaaggaatgcagctcatttcagaaaagcctgagacggaagctgtggtgaaggagaaactcactggtttacataaaatgtgggaagtccttgaatccactacccagacaaaggcccagcggctctttgatgcaaacaaggccgaacttttcacccagagctgtgcagatctagacaaatggctgcacggcctggagagtcagattcagtctgatgactatggcaaagacctgaccagtgtcaatatcctgctgaaaaagcaacagatgctggagaatcagatggaagtgcggaagaaggagatcgaagagctccaaagccaagcccaggccctgagtcaggaagggaagagcaccgacgaggtagacagcaagcgcctcaccgtgcagaccaagttcatggagttgctggagcccttgaacgagaggaagcataacctgctggcctccaaagagatccatcagttcaacagggatgtggaggacgagatcttgtgggttggagagaggatgcctttggcaacttccacggatcatggccacaacctccagactgtgcagctgttaataaagaaaaatcagaccctccagaaagaaatccaggggcaccagcctcgcattgacgacatctttgagaggagccaaaacatcgtcactgacagcagcagcctcagcgctgaggccatcagacagaggcttgccgacctgaagcagctgtggggtctcctcattgaggagacagagaaacgccacaggcggctggaggaggcgcacagggcccagcagtactactttgacgctgctgaggccgaagcctggatgagcgagcaggagctgtacatgatgtcagaggagaaggccaaggatgagcagagtgctgtctccatgttgaagaagcaccagatcttagaacaagctgtggaggactatgcagagaccgtgcatcagctctccaagaccagccgggccctggtggccgacagccatcctgaaagtgagcgcattagcatgcggcagtccaaagtggataaactgtacgctggtctgaaagaccttgctgaagagagaagaggcaagctggatgagagacacaggttattccagctcaaccgggaggtggacgacctggagcagtggatcgctgagagggaggtggtcgcagggtcccatgaactgggacaggactatgagcatgtcacgatgttacaagaacgattccgggagtttgcccgagacaccgggaacattgggcaggagcgcgtggacacggtcaatcacctggcagatgagctcatcaactctggacattcagatgccgccaccatcgctgaatggaaggatggcctcaatgaagcctgggccgacctcctggagctcattgacacaagaacacagattcttgccgcttcctatgaactgcacaagttttaccacgatgccaaggagatctttgggcgtatacaggacaaacacaagaaactccctgaggagcttgggagagatcagaacacagtggagaccttacagagaatgcacactacatttgagcatgacatccaggctctgggcacacaggtgaggcagctgcaggaggatgcagcccgcctccaggcggcctatgcgggtgacaaggccgacgatatccagaagcgcgagaacgaggtcctggaagcctggaagtccctcctggacgcctgtgagagccgcagggtgcggctggtggacacaggggacaagttccgcttcttcagcatggtgcgcgacctcatgctctggatggaggatgtcatccggcagatcgaggcccaggagaagccaagggatgtatcatctgttgaactcttaatgaataatcatcaaggcatcaaagctgaaattgatgcacgtaatgacagtttcacaacctgcattgaacttgggaaatccctgttggcgagaaaacactatgcatctgaggagatcaaggaaaaattactgcagttgacggaaaagaggaaagaaatgatcgacaagtgggaagaccgatgggaatggttaagactgattctggaggtccatcagttctcaagagacgccagtgtggccgaggcctggctgcttggacaggagccgtacctatccagccgagagataggccagagcgtggacgaggtggagaagctcatcaagcgccacgaggcatttgaaaagtctgcagcaacctgggatgagaggttctctgccctggaaaggctgactacattggagttactggaagtgcgcagacagcaagaggaagaggagaggaagaggcggccgccttctcccgagccgagcacgaaggtttcagaggaagccgagtcccagcagcagtgggatacttcaaaaggagaacaagtttcccaaaacggtttgccagctgaacagggatctccacggatggcagaaacggtggacacaagcgaaatggtcaacggcgctacagaacaaaggacgagctctaaagagtccagccccatcccctccccgacctctgatcgtaaagccaagactgccctcccagcccagagtgccgccaccttaccagccagaacccaggagacaccttcggcccagatggaaggcttcctcaatcggaaacacgagtgggaggcccacaataagaaagcctcaagcaggtcctggcacaatgtttattgtgtcataaataaccaagaaatgggtttctacaaagatgcaaagactgctgcttctggaattccctaccacagcgaggtccctgtgagtttgaaagaagctgtctgcgaagtggcccttgattacaaaaagaagaaacacgtattcaagctaagactaaatgatggcaatgagtacctcttccaagccaaagacgatgaggaaatgaacacatggatccaggctatctcttccgccatctcctctgataaacacgaggtgtctgccagcacccagagcacgccagcatccagccgcgcgcagaccctccccaccagcgtcgtcaccatcaccagcgagtccagtcccggcaagcgggaaaaggacaaagagaaagacaaagagaagcggttcagcctttttggcaaaaagaaatga

SEQ ID NO: 2 - DNA, 6465 bp, Artificial Sequence
  Homo sapiens spectrin beta, non-erythrocytic 1 (SPTBN1), transcript variant 2
atggaattgcagaggacgtctagtatctccgggccgctgtcgccggcgtacacggggcaggtgccttacaactacaaccagctggaaggcagattcaagcagctgcaagatgagcgtgaagccgtgcagaagaagaccttcaccaagtgggtcaattcccaccttgcccgtgtgtcctgccggatcacagacctgtacactgaccttcgagatggacggatgctcatcaagctgctggaggtcctctctggagagaggctgcctaaacccaccaagggacgaatgcgcatccactgcttagagaatgtggacaaggcccttcagttcctgaaggagcagagagtccatcttgagaacatggggtcccatgacatcgtggatggaaaccaccggctgacccttggcctcatctggaccatcatcctgcgcttccagatccaggatatcagtgtggaaactgaagacaacaaagagaagaaatctgccaaggatgcattgctgttgtggtgccagatgaagacagctgggtaccccaatgtcaacattcacaatttcaccactagctggagggacggcatggccttcaatgcactgatacacaaacaccggcctgacctgatagattttgacaaactaaagaaatctaacgcacactacaacctgcagaatgcatttaatctggcagaacagcacctcggcctcactaaactgttggaccccgaagacatcagcgtggaccatcctgatgagaagtccataatcacttatgtggtgacttattaccactacttctctaagatgaaggccttagctgttgaaggaaaacgaattggaaaggtgcttgacaatgctattgaaacagaaaaaatgattgaaaagtatgaatcacttgcctctgaccttctggaatggattgaacaaaccatcatcattctgaacaatcgcaaatttgccaattcactggtcggggttcaacagcagcttcaggcattcaacacttaccgcactgtggagaaaccacccaaatttactgagaaggggaacttggaagtgctgctcttcaccattcagagcaagatgagggccaacaaccagaaggtctacatgccccgggaggggaagctcatctctgacatcaacaaggcctgggaaagactggaaaaagcggaacacgaaagagaactggctttgcggaatgagctcataagacaggagaaactggaacagctcgcccgcagatttgatcgcaaggcagctatgagggagacttggctgagcgaaaaccagcgtctggtgtctcaggacaactttgggtttgaccttcctgcagttgaggccgccacaaaaaagcacgaggccattgagacagacattgccgcatacgaggagcgtgtgcaggctgtggtagccgtggccagggagctcgaggccgagaattaccacgacatcaagcgcatcacagcgaggaaggacaatgtcatccggctctgggaatacctactggaactgctcagggcccggagacagcggctcgagatgaacctggggctgcagaagatattccaggaaatgctctacattatggactggatggatgaaatgaaggtgctagtattgtctcaagactatggcaaacacttacttggtgtggaagacctgttacagaagcacaccctggttgaagcagacattggcatccaggcagagcgggtgagaggtgtcaatgcctccgcccagaagttcgcaacagacggggaaggttacaagccctgtgacccccaggtgatccgagaccgcgtggcccacatggagttctgttatcaagagctttgccagctggcggctgagcgcagggcccgtctggaagagtcccgccgcctctggaagttcttctgggagatggcagaagaggaaggctggatacgggagaaggagaagatcctgtcctcggacgattacgggaaagacctgaccagcgtcatgcgcctgctcagcaagcaccgggcgttcgaggacgagatgagcggccgcagtggccactttgagcaggccatcaaggaaggcgaagacatgatcgcggaggagcacttcgggtcggagaagatccgtgagaggatcatttacatccgggagcagtgggccaacctagagcagctctcggccattcggaagaagcgcctggaggaggcctccctgctgcaccagttccaggcagatgctgatgacattgatgcctggatgctggacatcctcaagattgtctccagcagcgacgtgggccacgatgagtattccacacagtctctggtcaagaaacacaaggacgtggcggaagagatcgccaattacaggcccacccttgacacgctgcacgaacaagccagcgccctcccccaggagcatgccgagtctccagacgtgaggggcaggctgtcgggcatcgaggagcggtataaggaggtggcagagctgacgcggctgcggaagcaggcactccaggacactctggccctgtacaagatgttcagcgaggctgatgcctgtgagctctggatcgacgagaaggagcagtggctcaacaacatgcagatcccagagaagctggaggatctggaggtcatccagcacagatttgagagcctagaaccagaaatgaacaaccaggcttcccgggttgcagtggtgaaccagattgcacgccagctgatgcacagcggccacccaagtgagaaggaaatcaaagcccagcaggacaaactcaacacaaggtggagccagttcagagaactggttgacaggaagaaggatgccctcctgtctgccctgagcatccagaactaccacctcgagtgcaatgaaaccaaatcctggattcgggaaaagaccaaggtcatcgagtccacccaggacctgggcaatgacctggctggcgtcatggccctgcagcgcaagctgaccggcatggagcgggacttggtggccattgaggcaaagctgagtgacctgcagaaggaggcggagaagctggagtccgagcaccccgaccaggcccaggccatcctgtctcggctggccgagatcagcgacgtgtgggaggagatgaagaccaccctgaaaaaccgagaggcctccctgggagaggccagcaagctgcagcagttcctacgggacttggacgacttccagtcctggctctctaggacccagacagcgatcgcctcggaggacatgccaaacaccctgaccgaggctgagaagctgctcacgcagcacgagaacatcaagaacgagatcgacaactacgaggaggactaccagaagatgagggacatgggcgagatggtcacccaggggcagaccgatgcccagtacatgtttctgcggcagcggctgcaggccctggacactggatggaacgagctccacaagatgtgggagaacagacaaaatctcctatcccagtcacatgcctaccagcagttcctcagagacacgaagcaagccgaagcctttcttaacaaccaggagtatgttctggctcacactgaaatgcctaccaccttggaaggagctgaagcagcaattaaaaagcaagaggacttcatgaccaccatggacgccaatgaggagaagatcaatgctgtggtggagactggccggaggctggtgagcgatgggaacatcaactcagatcgcatccaggagaaggtggactctattgatgacagacataggaagaatcgtgagacagccagtgaacttttgatgaggttgaaggacaacagggatctacagaaattcctgcaagattgtcaagagctgtctctctggatcaatgagaagatgctcacagcccaggacatgtcttacgatgaagccagaaatctgcacagtaaatggttgaagcatcaagcatttatggcagaacttgcatccaacaaagaatggcttgacaaaatcgagaaggaaggaatgcagctcatttcagaaaagcctgagacggaagctgtggtgaaggagaaactcactggtttacataaaatgtgggaagtccttgaatccactacccagacaaaggcccagcggctctttgatgcaaacaaggccgaacttttcacccagagctgtgcagatctagacaaatggctgcacggcctggagagtcagattcagtctgatgactatggcaaagacctgaccagtgtcaatatcctgctgaaaaagcaacagatgctggagaatcagatggaagtgcggaagaaggagatcgaagagctccaaagccaagcccaggccctgagtcaggaagggaagagcaccgacgaggtagacagcaagcgcctcaccgtgcagaccaagttcatggagttgctggagcccttgaacgagaggaagcataacctgctggcctccaaagagatccatcagttcaacagggatgtggaggacgagatcttgtgggttggagagaggatgcctttggcaacttccacggatcatggccacaacctccagactgtgcagctgttaataaagaaaaatcagaccctccagaaagaaatccaggggcaccagcctcgcattgacgacatctttgagaggagccaaaacatcgtcactgacagcagcagcctcagcgctgaggccatcagacagaggcttgccgacctgaagcagctgtggggtctcctcattgaggagacagagaaacgccacaggcggctggaggaggcgcacagggcccagcagtactactttgacgctgctgaggccgaagcctggatgagcgagcaggagctgtacatgatgtcagaggagaaggccaaggatgagcagagtgctgtctccatgttgaagaagcaccagatcttagaacaagctgtggaggactatgcagagaccgtgcatcagctctccaagaccagccgggccctggtggccgacagccatcctgaaagtgagcgcattagcatgcggcagtccaaagtggataaactgtacgctggtctgaaagaccttgctgaagagagaagaggcaagctggatgagagacacaggttattccagctcaaccgggaggtggacgacctggagcagtggatcgctgagagggaggtggtcgcagggtcccatgaactgggacaggactatgagcatgtcacgatgttacaagaacgattccgggagtttgcccgagacaccgggaacattgggcaggagcgcgtggacacggtcaatcacctggcagatgagctcatcaactctggacattcagatgccgccaccatcgctgaatggaaggatggcctcaatgaagcctgggccgacctcctggagctcattgacacaagaacacagattcttgccgcttcctatgaactgcacaagttttaccacgatgccaaggagatctttgggcgtatacaggacaaacacaagaaactccctgaggagcttgggagagatcagaacacagtggagaccttacagagaatgcacactacatttgagcatgacatccaggctctgggcacacaggtgaggcagctgcaggaggatgcagcccgcctccaggcggcctatgcgggtgacaaggccgacgatatccagaagcgcgagaacgaggtcctggaagcctggaagtccctcctggacgcctgtgagagccgcagggtgcggctggtggacacaggggacaagttccgcttcttcagcatggtgcgcgacctcatgctctggatggaggatgtcatccggcagatcgaggcccaggagaagccaagggatgtatcatctgttgaactcttaatgaataatcatcaaggcatcaaagctgaaattgatgcacgtaatgacagtttcacaacctgcattgaacttgggaaatccctgttggcgagaaaacactatgcatctgaggagatcaaggaaaaattactgcagttgacggaaaagaggaaagaaatgatcgacaagtgggaagaccgatgggaatggttaagactgattctggaggtccatcagttctcaagagacgccagtgtggccgaggcctggctgcttggacaggagccgtacctatccagccgagagataggccagagcgtggacgaggtggagaagctcatcaagcgccacgaggcatttgaaaagtctgcagcaacctgggatgagaggttctctgccctggaaaggctgactacattggagttactggaagtgcgcagacagcaagaggaagaggagaggaagaggcggccgccttctcccgagccgagcacgaaggtttcagaggaagccgagtcccagcagcagtgggatacttcaaaaggagaacaagtttcccaaaacggtttgccagctgaacagggatctccacgggttagttaccgctctcaaacctaccaaaactacaaaaactttaatagcagacggacagccagtgaccagccatggtctggactg

SEQ ID NO: 3 - DNA, 7092 bp, Artificial Sequence
  Mus musculus spectrin beta, non-erythrocytic 1 (Sptbn1), transcript variant 1
atgacgaccacggtagccacagactatgacaacattgagatccagcagcagtacagtgatgtgaacaaccgctgggatgtggatgactgggacaatgagaacagctctgcaaggctctttgagcgctcccgcatcaaggccctggcagatgagcgtgaagctgtacagaagaagaccttcaccaagtgggtcaattcccaccttgcgagagtgtcctgccgaatcacagacctgtacacggaccttcgagatggacggatgctcatcaagctactggaggtcctctctggagagaggctgcctaaacccactaagggacggatgcggatccactgtctggagaatgtcgacaaggctcttcaattcctgaaagagcagagagtccatcttgagaacatgggctcccatgacattgtggatggaaaccaccggctgaccctcggcctcatctggacaattattctgcgcttccagatccaggatattagtgtggagactgaagataacaaagagaaaaagtctgctaaggatgcattgctgctgtggtgccagatgaagacagctgggtaccccaatgtcaacattcacaatttcaccactagctggagggatggcatggccttcaatgcactgatacataaacatcggcctgacctgatagattttgataaactgaagaaatctaatgcacactacaatctgcagaatgcatttaacctggcagagcagcaccttggcctcactaaactgttagaccctgaagatatcagtgtggaccaccctgatgagaagtctatcatcacatacgtggtgacttactaccactacttctccaagatgaaggccttggctgtcgaaggaaagcgcattggaaaggtgcttgataatgctatagaaacagagaaaatgattgagaagtacgagtcacttgcttctgaccttctggagtggattgaacaaaccatcatcatcctaaacaaccgcaaatttgctaattcactggttggggtccagcagcagctccaggcattcaacacgtaccgcacagtggagaaaccacctaagtttactgagaaggggaatttggaggtgctccttttcacaattcagagcaagatgcgagcgaataatcagaaggtctacatgccccgcgaggggaagctcatctctgacatcaacaaggcctgggaaagactggaaaaagcagaacatgagagagaactggctctgcggaatgagctcatacggcaggaaaaactggaacaactcgcccgaagatttgatcgcaaggcagctatgagggagacatggctgagtgaaaaccagcgtcttgtgtctcaggacaactttggatttgaccttcccgctgttgaggctgctaccaaaaaacacgaggccattgagacagacatcgctgcatatgaagaacgagttcaggccgtggtggctgtggccagggaacttgaagccgagaactaccatgacatcaagcgcatcacagcgaggaaggacaatgtcatccggctctgggaatacttgctggaactgctcagggccaggaggcagcgtcttgagatgaacctgggattgcaaaagatattccaggaaatgctttatattatggactggatggatgaaatgaaggtgctattgctgtctcaagactatggcaaacacttacttggtgttgaagacctgttacagaagcatgccctggttgaagcagacattgcaatccaagcagagcgtgtaagaggtgtgaatgcctctgcccagaagtttgcaacagatggggaaggctacaagccatgtgacccccaggtaattcgagaccgtgttgcccacatggagttctgctatcaagagctttgtcagctggctgccgagcgtagggctcgcctggaagagtcccgtcgcctctggaagttcttctgggagatggcagaagaggaaggctggatacgagagaaggagaagatcctgtcctctgatgattacgggaaagacttgaccagtgtcatgcgcctgctgagcaagcaccgggcatttgaggatgagatgagtggccgtagtggccattttgagcaggccattaaagaaggtgaagacatgattgcagaggaacactttggatcggaaaagatccgtgagagaatcatttatatccgggagcagtgggccaacctggaacagctctcagccattaggaagaagcgcctagaggaagcctcattactgcaccagttccaggctgatgctgatgatattgatgcttggatgttagatatactcaagattgtctccagcaatgatgtgggccatgatgagtactccacgcagtctctggtcaagaagcataaagatgtagcagaagagatcaccaactacaggcccactattgacacactgcatgagcaagccagtgcccttccacaagcacatgcagagtctccagatgtgaagggccggctggcaggaattgaggagcgctgcaaggagatggcagagttaacacggctaaggaagcaggctctgcaggacaccctggccctgtacaagatgttcagtgaggctgatgcctgtgagctctggattgacgagaaggagcagtggctcaacaacatgcagatcccagagaagctggaggacctggaagtcatccagcacagatttgagagcctagaaccagaaatgaacaaccaggcttcccgggttgctgtggtgaaccagattgcacggcagctgatgcacaatggccaccccagtgaaaaggaaatcagagctcagcaagacaaactcaacacgaggtggagtcagttcagagaactggtggacaggaaaaaggatgctcttctgtctgccctgagcatccagaactaccacctcgagtgcaatgaaaccaaatcctggatccgggagaagaccaaggtcatcgagtctacccaagaccttggcaatgacctggcaggtgtcatggccctgcagcgcaagctgactggcatggaacgagacttggtagccattgaggcgaagctgagtgacctgcagaaagaagctgagaagctggagtccgagcaccctgaccaggctcaagctatcctgtctcggctggccgagatcagtgatgtgtgggaggaaatgaagacaaccctgaagaaccgagaggcctccctgggagaggccagcaagctgcagcagtttctgcgggacttggacgacttccagtcttggctctccaggacccagactgctatcgcctcagaggacatgcccaataccctcactgaggcagagaagcttctcacacagcacgagaatatcaaaaatgagatcgacaattatgaggaagactaccagaagatgcgggacatgggcgagatggtcacccaggggcagactgatgcccagtatatgtttctgcggcagcggctgcaggccttagacactggctggaatgagctccacaaaatgtgggagaacaggcaaaacctcctctcccagtcccatgcctaccagcagttccttagggacaccaaacaagctgaagcttttcttaataaccaggagtatgttttggctcatactgaaatgcccaccaccctggaaggagctgaagcagccattaaaaagcaggaggacttcatgaccaccatggatgccaacgaggagaagatcaatgctgttgtggagactggccgaagactggtgagcgatgggaacatcaactccgaccgcatccaggagaaggtggactctattgacgacagacacaggaagaatcgagaagcagccagtgaacttctgatgaggttaaaggacaaccgtgatctacagaagttcctgcaagattgtcaagagctgtccctctggatcaatgaaaagatgcttacagctcaagacatgtcttatgatgaagccagaaatctgcacagtaaatggttaaagcatcaagcatttatggcggaacttgcatccaacaaagaatggcttgacaaaattgagaaggaaggaatgcagcttatttcagaaaagccagaaacagaagctgtggtaaaggaaaaactcactggtttacataaaatgtgggaagtccttgaatccacaacccagaccaaggcccagcggctctttgatgcaaataaggctgagcttttcacacaaagctgcgcagatcttgacaaatggctacatggcctggagagccagattcaatctgacgactatggcaaagaccttaccagtgtcaatattcttctgaaaaagcaacagatgctggagaatcagatggaagttcggaagaaagagatcgaggaactgcagagccaagcccaggcgctgagtcaggaggggaagagcacagatgaggtggacagcaaacgccttactgtgcagaccaagttcatggagcttctggagcccttgagtgagaggaagcataacctgttagcttccaaggagatccatcagttcaacagggatgtggaggacgaaatcctatgggttggcgagaggatgcctttggcaacttccacagatcatggccataaccttcaaactgtgcagctgttaataaagaaaaaccagaccctccagaaagaaatccagggacaccagcctcgtattgatgacatctttgagaggagtcaaaacatcatcacagatagcagcagcctcaatgccgaggctatcaggcagaggctcgctgacctgaagcagctgtgggggctcctcattgaggaaactgagaaacgccatagacggctggaggaggcacacaaggcgcagcagtactactttgatgcagctgaagccgaggcatggatgagtgaacaggagttgtacatgatgtctgaggaaaaggccaaggatgagcagagtgctgtctctatgttgaaaaagcaccagattttagagcaagctgttgaggactatgcagagacagtacaccagctctccaagactagccgggcgctggtggctgacagccatcccgaaagtgagcgtattagcatgcggcagtcaaaggtcgacaagctgtatgctggcctgaaggaccttgctgaggagaggagaggaaaacttgatgagaggcacaggctgttccagctcaacagagaggtggatgacctggaacagtggatcgctgagagggaagtggtcgcaggctcccatgagttgggacaggactatgagcatgtcacgatgttacaagaacggttccgagaatttgctcgagacacaggaaacattgggcaggagcgtgtggatacagttaataacatggcagatgaactcatcaactctggacattcagatgctgccaccattgctgagtggaaagatggtctcaatgaagcctgggctgacctcctggagctcattgacacaagaacacagattcttgctgcctcatatgaacttcataagttttaccatgatgccaaggagatctttggccgaatccaggacaaacacaagaaactccctgaggagcttggaagagatcaaaacactgtggaaactttacagagaatgcacaccacctttgagcacgacatccaagctctgggcactcaggtgaggcagctgcaggaggatgcagctcgcctccaggcagcctatgcaggggacaaggctgatgacatccagaagcgtgagaatgaggtcctggaagcctggaagtccctgctggatgcttgtgagggtcgcagggtgcggctggtagacacaggagacaagttccgcttcttcagcatggtgcgtgacctcatgctctggatggaagatgtcatccggcagatcgaggcccaggagaaaccacgggatgtgtcatctgttgaactgttaatgaataatcatcaaggtatcaaagctgaaattgatgctcgtaatgacagctttacagcctgcattgagcttgggaaatccctgctggcacggaaacactatgcttctgaggagatcaaggaaaagttactgcagctgacagagaaaagaaaagaaatgattgacaagtgggaagaccggtgggagtggttaagactgattttggaggtccatcagttctcaagggatgccagtgtggcagaggcttggctgcttggacaggaaccatacctatccagccgtgaaattggccagagtgtagacgaagtggagaagcttattaagcgccatgaggcgtttgaaaagtctgcagcgacctgggatgagagattctctgctctggaaaggctgacaacgttggagctactggaagtgcgcagacagcaagaggaagaagaaagaaagaggcggccaccttctccggacccaaacacgaaggtttcagaggaggctgagtcccagcaatgggatacttcaaaaggagaccaagtttcccagaatggtttgccggctgagcagggatctccacggatggcaggaaccatggaaacgagtgaaatggtcaacggtgctgctgagcagaggacaagctccaaagagtccagtcctgttccctctcccaccttggaccgaaaggccaaatctgcacttccagcccagagtgctgccaccctgccagccaggaccctggagacacccgctgcccagatggaaggcttcctcaatcggaagcatgagtgggaggcccacaataagaaagcctcgagcaggtcctggcacaatgtatattgtgtcataaataaccaagaaatgggcttctataaagatgccaagagtgctgcttctggcatcccctaccacagtgaggtccctgtgagtttgaaagaggccatctgcgaagtggcccttgattacaaaaagaagaagcacgtgttcaagctaagactaagtgatggaaacgagtacctcttccaagccaaagatgatgaggaaatgaacacatggatccaggctatctcctctgccatctcctctgacaaacacgacacatctgccagcacccagagtacgccagcatccagtcgggcgcagaccttacccaccagcgtcgtcaccatcaccagcgagtccagtcctggcaagagggagaaggataaagagaaagacaaagagaagaggttcagccttttcggcaagaagaagtga

SEQ ID NO: 4 - DNA, 6462 bp, Artificial Sequence
  Mus musculus spectrin beta, non-erythrocytic 1 (Sptbn1), transcript variant 2
atggagttgcagaggacatccagcatttcagggccgctgtcgccggcctacaccgggcaggtgccttacaactacaaccaactggaaggaagattcaaacagctccaagatgagcgtgaagctgtacagaagaagaccttcaccaagtgggtcaattcccaccttgcgagagtgtcctgccgaatcacagacctgtacacggaccttcgagatggacggatgctcatcaagctactggaggtcctctctggagagaggctgcctaaacccactaagggacggatgcggatccactgtctggagaatgtcgacaaggctcttcaattcctgaaagagcagagagtccatcttgagaacatgggctcccatgacattgtggatggaaaccaccggctgaccctcggcctcatctggacaattattctgcgcttccagatccaggatattagtgtggagactgaagataacaaagagaaaaagtctgctaaggatgcattgctgctgtggtgccagatgaagacagctgggtaccccaatgtcaacattcacaatttcaccactagctggagggatggcatggccttcaatgcactgatacataaacatcggcctgacctgatagattttgataaactgaagaaatctaatgcacactacaatctgcagaatgcatttaacctggcagagcagcaccttggcctcactaaactgttagaccctgaagatatcagtgtggaccaccctgatgagaagtctatcatcacatacgtggtgacttactaccactacttctccaagatgaaggccttggctgtcgaaggaaagcgcattggaaaggtgcttgataatgctatagaaacagagaaaatgattgagaagtacgagtcacttgcttctgaccttctggagtggattgaacaaaccatcatcatcctaaacaaccgcaaatttgctaattcactggttggggtccagcagcagctccaggcattcaacacgtaccgcacagtggagaaaccacctaagtttactgagaaggggaatttggaggtgctccttttcacaattcagagcaagatgcgagcgaataatcagaaggtctacatgccccgcgaggggaagctcatctctgacatcaacaaggcctgggaaagactggaaaaagcagaacatgagagagaactggctctgcggaatgagctcatacggcaggaaaaactggaacaactcgcccgaagatttgatcgcaaggcagctatgagggagacatggctgagtgaaaaccagcgtcttgtgtctcaggacaactttggatttgaccttcccgctgttgaggctgctaccaaaaaacacgaggccattgagacagacatcgctgcatatgaagaacgagttcaggccgtggtggctgtggccagggaacttgaagccgagaactaccatgacatcaagcgcatcacagcgaggaaggacaatgtcatccggctctgggaatacttgctggaactgctcagggccaggaggcagcgtcttgagatgaacctgggattgcaaaagatattccaggaaatgctttatattatggactggatggatgaaatgaaggtgctattgctgtctcaagactatggcaaacacttacttggtgttgaagacctgttacagaagcatgccctggttgaagcagacattgcaatccaagcagagcgtgtaagaggtgtgaatgcctctgcccagaagtttgcaacagatggggaaggctacaagccatgtgacccccaggtaattcgagaccgtgttgcccacatggagttctgctatcaagagctttgtcagctggctgccgagcgtagggctcgcctggaagagtcccgtcgcctctggaagttcttctgggagatggcagaagaggaaggctggatacgagagaaggagaagatcctgtcctctgatgattacgggaaagacttgaccagtgtcatgcgcctgctgagcaagcaccgggcatttgaggatgagatgagtggccgtagtggccattttgagcaggccattaaagaaggtgaagacatgattgcagaggaacactttggatcggaaaagatccgtgagagaatcatttatatccgggagcagtgggccaacctggaacagctctcagccattaggaagaagcgcctagaggaagcctcattactgcaccagttccaggctgatgctgatgatattgatgcttggatgttagatatactcaagattgtctccagcaatgatgtgggccatgatgagtactccacgcagtctctggtcaagaagcataaagatgtagcagaagagatcaccaactacaggcccactattgacacactgcatgagcaagccagtgcccttccacaagcacatgcagagtctccagatgtgaagggccggctggcaggaattgaggagcgctgcaaggagatggcagagttaacacggctaaggaagcaggctctgcaggacaccctggccctgtacaagatgttcagtgaggctgatgcctgtgagctctggattgacgagaaggagcagtggctcaacaacatgcagatcccagagaagctggaggacctggaagtcatccagcacagatttgagagcctagaaccagaaatgaacaaccaggcttcccgggttgctgtggtgaaccagattgcacggcagctgatgcacaatggccaccccagtgaaaaggaaatcagagctcagcaagacaaactcaacacgaggtggagtcagttcagagaactggtggacaggaaaaaggatgctcttctgtctgccctgagcatccagaactaccacctcgagtgcaatgaaaccaaatcctggatccgggagaagaccaaggtcatcgagtctacccaagaccttggcaatgacctggcaggtgtcatggccctgcagcgcaagctgactggcatggaacgagacttggtagccattgaggcgaagctgagtgacctgcagaaagaagctgagaagctggagtccgagcaccctgaccaggctcaagctatcctgtctcggctggccgagatcagtgatgtgtgggaggaaatgaagacaaccctgaagaaccgagaggcctccctgggagaggccagcaagctgcagcagtttctgcgggacttggacgacttccagtcttggctctccaggacccagactgctatcgcctcagaggacatgcccaataccctcactgaggcagagaagcttctcacacagcacgagaatatcaaaaatgagatcgacaattatgaggaagactaccagaagatgcgggacatgggcgagatggtcacccaggggcagactgatgcccagtatatgtttctgcggcagcggctgcaggccttagacactggctggaatgagctccacaaaatgtgggagaacaggcaaaacctcctctcccagtcccatgcctaccagcagttccttagggacaccaaacaagctgaagcttttcttaataaccaggagtatgttttggctcatactgaaatgcccaccaccctggaaggagctgaagcagccattaaaaagcaggaggacttcatgaccaccatggatgccaacgaggagaagatcaatgctgttgtggagactggccgaagactggtgagcgatgggaacatcaactccgaccgcatccaggagaaggtggactctattgacgacagacacaggaagaatcgagaagcagccagtgaacttctgatgaggttaaaggacaaccgtgatctacagaagttcctgcaagattgtcaagagctgtccctctggatcaatgaaaagatgcttacagctcaagacatgtcttatgatgaagccagaaatctgcacagtaaatggttaaagcatcaagcatttatggcggaacttgcatccaacaaagaatggcttgacaaaattgagaaggaaggaatgcagcttatttcagaaaagccagaaacagaagctgtggtaaaggaaaaactcactggtttacataaaatgtgggaagtccttgaatccacaacccagaccaaggcccagcggctctttgatgcaaataaggctgagcttttcacacaaagctgcgcagatcttgacaaatggctacatggcctggagagccagattcaatctgacgactatggcaaagaccttaccagtgtcaatattcttctgaaaaagcaacagatgctggagaatcagatggaagttcggaagaaagagatcgaggaactgcagagccaagcccaggcgctgagtcaggaggggaagagcacagatgaggtggacagcaaacgccttactgtgcagaccaagttcatggagcttctggagcccttgagtgagaggaagcataacctgttagcttccaaggagatccatcagttcaacagggatgtggaggacgaaatcctatgggttggcgagaggatgcctttggcaacttccacagatcatggccataaccttcaaactgtgcagctgttaataaagaaaaaccagaccctccagaaagaaatccagggacaccagcctcgtattgatgacatctttgagaggagtcaaaacatcatcacagatagcagcagcctcaatgccgaggctatcaggcagaggctcgctgacctgaagcagctgtgggggctcctcattgaggaaactgagaaacgccatagacggctggaggaggcacacaaggcgcagcagtactactttgatgcagctgaagccgaggcatggatgagtgaacaggagttgtacatgatgtctgaggaaaaggccaaggatgagcagagtgctgtctctatgttgaaaaagcaccagattttagagcaagctgttgaggactatgcagagacagtacaccagctctccaagactagccgggcgctggtggctgacagccatcccgaaagtgagcgtattagcatgcggcagtcaaaggtcgacaagctgtatgctggcctgaaggaccttgctgaggagaggagaggaaaacttgatgagaggcacaggctgttccagctcaacagagaggtggatgacctggaacagtggatcgctgagagggaagtggtcgcaggctcccatgagttgggacaggactatgagcatgtcacgatgttacaagaacggttccgagaatttgctcgagacacaggaaacattgggcaggagcgtgtggatacagttaataacatggcagatgaactcatcaactctggacattcagatgctgccaccattgctgagtggaaagatggtctcaatgaagcctgggctgacctcctggagctcattgacacaagaacacagattcttgctgcctcatatgaacttcataagttttaccatgatgccaaggagatctttggccgaatccaggacaaacacaagaaactccctgaggagcttggaagagatcaaaacactgtggaaactttacagagaatgcacaccacctttgagcacgacatccaagctctgggcactcaggtgaggcagctgcaggaggatgcagctcgcctccaggcagcctatgcaggggacaaggctgatgacatccagaagcgtgagaatgaggtcctggaagcctggaagtccctgctggatgcttgtgagggtcgcagggtgcggctggtagacacaggagacaagttccgcttcttcagcatggtgcgtgacctcatgctctggatggaagatgtcatccggcagatcgaggcccaggagaaaccacgggatgtgtcatctgttgaactgttaatgaataatcatcaaggtatcaaagctgaaattgatgctcgtaatgacagctttacagcctgcattgagcttgggaaatccctgctggcacggaaacactatgcttctgaggagatcaaggaaaagttactgcagctgacagagaaaagaaaagaaatgattgacaagtgggaagaccggtgggagtggttaagactgattttggaggtccatcagttctcaagggatgccagtgtggcagaggcttggctgcttggacaggaaccatacctatccagccgtgaaattggccagagtgtagacgaagtggagaagcttattaagcgccatgaggcgtttgaaaagtctgcagcgacctgggatgagagattctctgctctggaaaggctgacaacgttggagctactggaagtgcgcagacagcaagaggaagaagaaagaaagaggcggccaccttctccggacccaaacacgaaggtttcagaggaggctgagtcccagcaatgggatacttcaaaaggagaccaagtttcccagaatggtttgccggctgagcagggatctccacgggttagttaccgctctcaaacgtaccaaaactacaaaaactttaatagcagacggacagccagtgaccattcatggtctggaatg

SEQ ID NO: 5 - DNA, 21 bp, Artificial Sequence
  Mouse siRNA Targeting Sptbn1 Sense Sequence
cgauguuacaagaacgguutt

SEQ ID NO: 6 - DNA, 21 bp, Artificial Sequence
  Mouse siRNA Targeting Sptbn1 Antisense Sequence
aaccguucuuguaacaucgtg

SEQ ID NO: 7 - RNA, 19 nt, Artificial Sequence
  Human siRNA target Sequence
ccugaaagugagcgcauua

SEQ ID NO: 8 - RNA, 19 nt, Artificial Sequence
  Human siRNA target Sequence
ccgcauacgaggagcgugu

SEQ ID NO: 9 - RNA, 19 nt, Artificial Sequence
  Human siRNA target Sequence
ggacaugucuuacgaugaa

SEQ ID NO: 10 - RNA, 19 nt, Artificial Sequence
  Human siRNA target Sequence
gugacaaggccgacgauau

(19) United States
(12) Patent Application Publication
(10) Pub. No.: US 2023/0416739 A1
(43) Pub. Date: Dec. 28, 2023

(54) B-SPECTRIN (SPTBN1) DEFICIENCY PROTECTS MICE FROM HIGH-FAT DIET-INDUCED LIVER DISEASE AND CANCER DEVELOPMENT

(71) Applicant: The George Washington University, A Congressionally Chartered Not-For-Profit Corporation, Washington, DC (US)

(72) Inventors: Lopa MISHRA, Washington, DC (US); Shuyun RAO, Vienna, VA (US); Wilma JOGUNOORI, Fairfax, VA (US); Bibhuti MISHRA, Washington, DC (US); Kazufumi OHSHIRO, Washington, DC (US); Sobia ZAIDI, Washington, DC (US)

(73) Assignee: The George Washington University, A Congressionally Chartered Not-For-Profit Corporation, Washington, DC (US)

(21) Appl. No.: 18/252,771

(22) PCT Filed: Nov. 12, 2021
(86) PCT No.: PCT/US2021/059245

§ 371 Date: May 12, 2023

Related U.S. Application Data
(60) Provisional application No. 63/147,141, filed on Feb. 8, 2021.
(60) Provisional application No. 63/113,745, filed on Nov. 13, 2020.

(51) Int. Cl. C12N 15/113; A61P 1/16

(52) U.S. Cl. CPC C12N 15/113; A61P 1/16; C12N 2310/14

(57) ABSTRACT

This disclosure provides methods of treating obesity, nonalcohol-related fatty liver disease, nonalcoholic steatohepatitis, or hepatocellular cancer in a subject in need thereof, the method comprising administering a therapeutically effective amount of at least one siRNA molecule targeting SPTBN1.

### GOVERNMENT SUPPORT

This invention was made with government support under U01 CA230690 and R01 M023146 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3973_018PC02_Seglisting_ST25.txt; Size: 37,736 bytes; and Date of Creation: Nov. 3, 2021) filed with the application is herein incorporated by reference in its entirety.

### TECHNICAL FIELD

This disclosure provides methods of treating obesity, nonalcohol-related fatty liver disease, nonalcoholic steatohepatitis, or hepatocellular cancer in a subject in need thereof, the method comprising administering a therapeutically effective amount of an siRNA molecule targeting SPTBN1.

### BACKGROUND OF THE INVENTION

Nonalcohol-related fatty liver diseases (NAFLD) and nonalcoholic steatohepatitis (NASH) arise from obesity and metabolic disorders and affect up to a third of the world's population (1, 2). These diseases comprise a spectrum including lipid accumulation in the liver (steatosis), injury, inflammation, hepatocyte ballooning (cell death), and progressive fibrosis (cirrhosis), ultimately leading to carcinogenesis (3, 4). Lipid accumulation in the liver promotes chronic oxidative and endoplasmic reticulum (ER) stress, cell death, immune cell infiltration, fibrogenesis, and disease progression. These effects are exacerbated by factors such as increased intake of free fatty acids (FFAs), sedentary lifestyle, and hyperinsulinemia. Depending on whether NASH occurs with or without cirrhosis, liver cancer incidence can vary from 2.4% (without cirrhosis) to 12.8% (with cirrhosis) (1, 3). Obesity increases the risk of liver cancer mortality twofold, and together with NASH, accounts for the alarming increase in this cancer (5, 6). Despite new therapeutic approaches targeting NASH, few single agents reverse both fibrosis and steatosis, thus NASH presents a major clinical challenge (5, 7). Therefore, understanding the molecular mechanisms that converge on abnormal lipid accumulation, fibrosis, and the fatal switch to hepatocarcinogenesis could lead to new approaches targeting NASH in specific groups susceptible to progression of disease.

Initiation of NAFLD is considered to involve de novo lipogenesis with abnormal accumulation of free fatty acids, triglycerides, and cholesterol. Activation of hepatocyte death receptor pathways, tumor necrosis factor (TNF), and caspases contribute to tissue injury and steatohepatitis observed in NASH (8, 9). De novo lipogenesis is stimulated by activation of the transcription factors sterol regulatory element (SRE)-binding proteins (SREBPs) and repression of energy-sensing pathways, such as the pathway involving adenosine monophosphate (AMP)-activated protein kinase (AMPK). SREBP1 is a master lipogenic transcriptional factor driving fatty acid synthesis and contributing to liver steatosis (10). SREBP proteins are maintained in the endoplasmic reticulum through interactions with the proteins INSIG and SCAP. SREBP activation in response to sterol depletion or ER stress requires disassociation of INSIG and SCAP-induced cleavage of SREBP by site-1 protease (S1P) followed by a second cleavage by site-2 protease (S2P) to produce the mature form of the SREBP proteins that translocate into the nucleus and regulate target gene transcription (11). In stressed cultured cells with activated caspase-3, SREBP1 and SREBP2 are cleaved and activated by caspase-3, but the physiological context for this is unknown (12). The cleaved, nuclear form is referred as n-SREBP and the full-length, ER-localized form as pre-SREBP.

The degree of fibrosis is considered the strongest predictive factor for progression of NALFD to NASH and ultimately hepatocellular cancer (HCC) (1, 13, 14). Critical to hepatic fibrosis is activation of the transforming growth factor β (TGF-β) pathway (15, 16). TGF-β1 is the founding member of this family, and this ligand signals through two serine-threonine kinase receptors (TGFBR2 and TGFBR1), which activate the SMAD transcriptional regulators. SMAD complexes containing SMAD3 serving as central to progression of fibrosis by causing excessive extracellular matrix gene expression, such as those encoding collagens COL1A1, COL1A2, COL3A1, COL5A2, COL6A1, and COL6A3, and stimulating genes encoding the protease inhibitors tissue inhibitor of metalloproteinases (TIMP) and plasminogen activator inhibitor-1 (PAI-1) (16). SMAD3 complexes also repress the gene encoding peroxisome proliferator-activated receptor gamma coactivator 1α (PGC-α) (17). Pro-fibrotic effects of TGF-β1 through SMAD3 involve multiple mechanisms and cell types, including enhanced infiltration or proliferation (or both) of tissue-resident fibroblasts, generation of myofibroblasts, induction of epithelial-mesenchymal transition (EMT), and inhibition of collagen lysis (18).

SPTBN1 (also called β2-spectrin, β2SP) is multidomain adaptor protein with functions in the cytoplasm and nucleus (19-21). In particular, SPTBN1 promotes TGF-β receptor activation of SMAD3 in the cytosol (22) and interacts with SMAD3 in the nucleus to regulate specific target genes (23, 24). SPTBN1 is a dynamic, tetrameric protein consisting of two antiparallel dimers of alpha and beta subunits. SPTBN1 binding partners include ankyrin, which functions to connect proteins at the cell membrane to the spectrin-containing cytoskeleton, and lamins and the chromatin modulator CTCF (CCCTC-binding factor), which function in the nucleus to organize chromatin and regulate gene expression (19, 23, 25, 26). SPTBN1 is a substrate for caspase-3 and -7, and cleavage at the SPTBN1 1454DEVD1457 peptide sequence produces two fragments (160 and 80 kDa) with distinct and separate functions in apoptosis and transcription (27). The importance of SPTBN1 in liver disease arises from finding that mice treated with shRNA targeting SPTBN1 exhibit less acetaminophen-induced hepatotoxicity (27).

Increased amounts of liver SMAD3 and SPTBN1, as well as TGF-β pathway members associated with pro-fibrotic pathways, are observed in ˜40% of HCCs, and many HCCs are associated with NASH (24, 28).

### SUMMARY OF THE INVENTION

The present disclosure provides methods of treating a disease, disorder, or condition, e.g., obesity, nonalcohol-related fatty liver disease, nonalcoholic steatohepatitis, or hepatocellular cancer, in a subject in need thereof, the method comprising administering a therapeutically effective amount of at least one siRNA molecule that inhibits expression of SPTBN1.

### DETAILED DESCRIPTION OF THE INVENTION

The role of SPTBN1 in liver tumorigenesis was investigated by generating liver-specific SPTBN1 conditional knockout (LSKO) mice. LSKO mice or mice treated with SPTBN1-targeted siRNA were shown to be protected from detrimental effects of a high-fat diet through a mechanism involving reduction of the expression of pro-fibrosis genes and genes involved in de novo lipogenesis. The mice did not become obese or develop NASH or HCC. The translational importance of the results was confirmed by analysis of the expression of SPTBN1 in human NASH and HCC and finding that siRNA targeting SPTBN1 reversed transcriptional changes in genes involved in fatty acid metabolism and fibrosis induced in a human 3D culture model of NASH. Thus, the results identified a previously unknown role for SPTBN1 in regulating SREBP activity induced by caspase-3 in response to stress conditions caused by a high-fat diet (HFD).

In one embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1.

In another embodiment, at least one of the siRNA molecules that inhibits expression of SPTBN1 comprises 15 to 30 nucleotides.

In another embodiment, at least one of the siRNA molecules that inhibits expression of SPTBN1 comprises 15 to 20 nucleotides.

In another embodiment, at least one of the siRNA molecules that inhibits expression of SPTBN1 comprises an overhang region of 1 to 6 nucleotides.

In another embodiment, at least one of the siRNA molecules that inhibits expression of SPTBN1 is comprises no overhang region.

In another embodiment, at least one of the siRNA molecules that inhibits expression of SPTBN1 is set forth as SEQ ID No. 7.

In another embodiment, at least one of the siRNA molecules that inhibits expression of SPTBN1 is set forth as SEQ ID No. 8.

In another embodiment, at least one of the siRNA molecules that inhibits expression of SPTBN1 is set forth as SEQ ID No. 9.

In another embodiment, at least one of the siRNA molecules that inhibits expression of SPTBN1 is set forth as SEQ ID No. 10.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 is homologous with at least 10 nucleotides of SEQ ID No. 7. In another embodiment, at least one of the siRNA molecules that inhibits expression of SPTBN1 is homologous with at least 15 nucleotides of SEQ ID No. 7.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 is homologous with at least 10 nucleotides of SEQ ID No. 8. In another embodiment, at least one of the siRNA molecules that inhibits expression of SPTBN1 is homologous with at least 15 nucleotides of SEQ ID No. 8.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 is homologous with at least 10 nucleotides of SEQ ID No. 9. In another embodiment, at least one of the siRNA molecules that inhibits expression of SPTBN1 is homologous with at least 15 nucleotides of SEQ ID No. 9.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 is homologous with at least 10 nucleotides of SEQ ID No. 10. In another embodiment, at least one of the siRNA molecules that inhibits expression of SPTBN1 is homologous with at least 15 nucleotides of SEQ ID No. 10.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 has at least 90% sequence identity with SEQ ID No. 7. In another embodiment, at least one of the siRNA molecules that inhibits expression of SPTBN1 has at least 95% sequence identity with SEQ ID No. 7.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 has at least 90% sequence identity with SEQ ID No. 8. In another embodiment, at least one of the siRNA molecules that inhibits expression of SPTBN1 has at least 95% sequence identity with SEQ ID No. 8.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 has at least 90% sequence identity with SEQ ID No. 9. In another embodiment, at least one of the siRNA molecules that inhibits expression of SPTBN1 has at least 95% sequence identity with SEQ ID No. 9.

In another embodiment, the disclosure provides a method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 has at least 90% sequence identity SEQ ID No. 10. In another embodiment, at least one of the siRNA molecules that inhibits expression of SPTBN1 has at least 95% sequence identity with SEQ ID No. 10.

In another embodiment, the disclosure provides a methods for treating obesity in a subject in need thereof. In another embodiment, treating obesity comprises reducing the amount of body fat in the subject and/or reducing the body weight of the subject.

In another embodiment, the disclosure provides a methods for treating nonalcohol-related fatty liver disease in a subject in need thereof. In another embodiment, the treating nonalcohol-related fatty liver disease comprises reducing blood triglycerides in the subject.

In another embodiment, the disclosure provides a methods for treating nonalcoholic steatohepatitis in a subject in need thereof. In another embodiment, the treating nonalcoholic steatohepatitis comprises reducing blood triglycerides in the subject.

In another embodiment, the disclosure provides a methods for treating hepatocellular cancer in a subject in need thereof. In another embodiment, treating hepatocellular cancer comprises reducing tumor mass in the subject.

In another embodiment, one to ten siRNA molecules, e.g, one to five siRNA molecules, e.g., one to three siRNA molecules, that inhibit(s) expression of SPTBN1 are administered to the subject. In another embodiment, one siRNA molecule that inhibits expression of SPTBN1 is administered to the subject. In another embodiment, two siRNA molecules that inhibit expression of SPTBN1 are administered to the subject. In another embodiment, three siRNA molecules that inhibit expression of SPTBN1 are administered to the subject. In another embodiment, four siRNA molecules that inhibit expression of SPTBN1 are administered to the subject. In another embodiment, five siRNA molecules that inhibit expression of SPTBN1 are administered to the subject. In another embodiment, six siRNA molecules that inhibit expression of SPTBN1 are administered to the subject. In another embodiment, seven siRNA molecules that inhibit expression of SPTBN1 are administered to the subject. In another embodiment, eight siRNA molecules that inhibit expression of SPTBN1 are administered to the subject. In another embodiment, nine siRNA molecules that inhibit expression of SPTBN1 are administered to the subject. In another embodiment, ten siRNA molecules that inhibit expression of SPTBN1 are administered to the subject.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of the therapeutic agent, e.g., an siRNA molecule that inhibits expression of SPTBN1, sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that causes a therapeutic response, e.g., normalization of blood counts, decrease in the rate of tumor growth, decrease in tumor mass, decrease in the number of metastases, increase in time to tumor progression, and/or increase subject survival time by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more. With respect to the treatment of obesity, in one embodiment, a therapeutically effective amount will refer to the amount of a therapeutic agent that causes a reduction of body fat or weight in the subject by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more. With respect to the treatment of NAFLD or NASH, in one embodiment, a therapeutically effective amount will refer to the amount of a therapeutic agent that causes a reduce of blood triglycerides in the subject by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The terms “G,” “C,” “A,” “T,” and “U” each generally stand for a naturally-occurring nucleotide that contains guanine, cytosine, adenine, thymidine, and uracil as a base, respectively. However, it will be understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “nucleobase” and “base” include the purine (e.g. adenine and guanine) and pyrimidine (e.g. uracil, thymine, and cytosine) moiety present in nucleosides and nucleotides which form hydrogen bonds in nucleic acid hybridization. In the context of the present invention, the term nucleobase also encompasses alternative nucleobases which may differ from naturally-occurring nucleobases, but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are for example described in Hirao et al (2012) Accounts of Chemical Research vol 45 page 2055 and Bergstrom (2009) Current Protocols in Nucleic Acid Chemistry Suppl. 37 1.4.1.

The term “nucleoside” refers to a monomeric unit of an oligonucleotide or a polynucleotide having a nucleobase and a sugar moiety. A nucleoside may include those that are naturally-occurring as well as alternative nucleosides, such as those described herein. The nucleobase of a nucleoside may be a naturally-occurring nucleobase or an alternative nucleobase. Similarly, the sugar moiety of a nucleoside may be a naturally-occurring sugar or an alternative sugar.

The term “alternative nucleoside” refers to a nucleoside having an alternative sugar or an alternative nucleobase, such as those described herein.

In a some embodiments the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine 5-thiazolo-uridine, 2-thio-uridine, pseudouridine, 1-methylpseudouridine, 5-methoxyuridine, 2′-thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g. A, T, G, C, or U, wherein each letter may optionally include alternative nucleobases of equivalent function.

A “sugar” or “sugar moiety,” includes naturally occurring sugars having a furanose ring. A sugar also include an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, 0-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein refers to a monomeric unit of an oligonucleotide or polynucleotide that comprises a nucleoside and an internucleosidic linkage. The internucleosidic linkage may or may not include a phosphate linkage. Similarly, “linked nucleosides” may or may not be linked by phosphate linkages. Many “alternative internucleosidic linkages” are known in the art, including, but not limited to, phosphate, phosphorothioate, and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide,” as used herein, refers to a nucleotide having an alternative nucleoside or an alternative sugar, and an internucleoside linkage, which may include alternative nucleoside linkages.

The terms “oligonucleotide” and “polynucleotide,” as used herein, are defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention may be man-made, and is chemically synthesized, and is typically purified or isolated. Oligonucleotide is also intended to include (i) compounds that have one or more furanose moieties that are replaced by furanose derivatives or by any structure, cyclic or acyclic, that may be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that may be used as a point of covalent attachment for the base moiety. The oligonucleotides of the invention may comprise one or more alternative nucleosides or nucleotides (e.g., including those described herein). It is also understood that oligonucleotide includes compositions lacking a sugar moiety or nucleobase but is still capable of forming a pairing with or hybridizing to a target sequence.

“Oligonucleotide” refers to a short polynucleotide (e.g., of 100 or fewer linked nucleosides).

As used herein, the term “strand” refers to an oligonucleotide comprising a chain of linked nucleosides. A “strand comprising a nucleobase sequence” refers to an oligonucleotide comprising a chain of linked nucleosides that is described by the sequence referred to using the standard nucleobase nomenclature.

The term “antisense,” as used herein, refers to a nucleic acid comprising an oligonucleotide or polynucleotide that is sufficiently complementary to all or a portion of a gene, primary transcript, or processed mRNA, so as to interfere with expression of the endogenous gene (e.g., MLH3). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The terms “antisense strand” and “guide strand” refer to the strand of a dsRNA that includes a region that is substantially complementary to a target sequence, e.g., an MLH3 mRNA.

The terms “sense strand” and “passenger strand,” as used herein, refer to the strand of a dsRNA that includes a region that is substantially complementary to a region of the antisense strand as that term is defined herein.

The term “dsRNA” refers to an agent that includes a sense strand and antisense strand that contains linked nucleosides as that term is defined herein. dsRNA includes, for example, siRNAs and shRNAs, which mediate the targeted cleavage of an RNA transcript via an RNA-induced silencing complex (RISC) pathway. dsRNA directs the sequence-specific degradation of mRNA through a process known as RNA interference (RNAi). The dsRNA reduces the expression of MLH3 in a cell, e.g., a cell within a subject, such as a mammalian subject. In general, the majority of linked nucleosides of each strand of a dsRNA are ribonucleosides, but as described in detail herein, each or both strands can also include one or more non-ribonucleosides, e.g., deoxyribonucleosides and/or alternative nucleosides.

The terms “siRNA” and “short interfering RNA” (also known as “small interfering RNA”) refer to an RNA agent, preferably a double-stranded agent, of about 10-50 nucleotides in length, the strands optionally having overhanging ends comprising, for example 1, 2 or 3 overhanging linked nucleosides, which is capable of directing or mediating RNA interference. Naturally-occurring siRNAs are generated from longer dsRNA molecules (e.g., >25 linked nucleosides in length) by a cell's RNAi machinery (e.g., Dicer or a homolog thereof).

The terms “shRNA” and “short hairpin RNA,” as used herein, refer to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleobases within the loop region.

“Chimeric” dsRNA or “chimeras,” in the context of this invention, is dsRNA which contain two or more chemically distinct regions, each made up of at least one monomer unit, i.e., a nucleoside or nucleotide in the case of a dsRNA.

The duplex region may be of any length that permits specific degradation of a desired target RNA through a RISC pathway, and may range from about 9 to 36 base pairs in length, e.g., about 10-30 base pairs in length, e.g., about 15-30 base pairs in length or about 18-20 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one longer oligonucleotide molecule, or they may be separate oligonucleotide molecules. Where the two strands are part of one larger molecule, and therefore are connected by an uninterrupted chain of linked nucleosides between the 3′-end of one strand and the 5′-end of the respective other strand forming the duplex structure, the connecting chain is referred to as a “hairpin loop.” A hairpin loop can comprise at least one unpaired nucleobase. In some embodiments, the hairpin loop can comprise at least 2, at least 3, at least 4, at least 5, at least 6, at least 7, at least 8, at least 9, at least 10, at least 20, at least 23 or more unpaired nucleobases. In some embodiments, the hairpin loop can be 10 or fewer linked nucleosides. In some embodiments, the hairpin loop can be 8 or fewer unpaired nucleobases. In some embodiments, the hairpin loop can be 4-10 unpaired nucleobases. In some embodiments, the hairpin loop can be 4-8 linked nucleosides.

In one embodiment, each strand of the dsRNA includes 19-23 linked nucleosides that interacts with a target RNA sequence, e.g., an MLH3 target mRNA sequence, to direct the cleavage of the target RNA. Without wishing to be bound by theory, long double stranded RNA introduced into cells is broken down by a Type III endonuclease known as Dicer (Sharp et al. (2001) Genes Dev. 15:485). Dicer, a ribonuclease-II-like enzyme, processes the RNA into 19-23 base pair short interfering RNAs with characteristic two-base 3′ overhangs (Bernstein, et al., (2001) Nature 409:363). The dsRNAs are then incorporated into an RNA-induced silencing complex (RISC) where one or more helicases unwind the dsRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Where the two substantially complementary strands of a dsRNA are comprised of separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.”

The term “linker” or “linking group” means an organic moiety that connects two parts of a compound, e.g., covalently attaches two parts of a compound. The RNA strands may have the same or a different number of linked nucleosides. The maximum number of base pairs is the number of linked nucleosides in the shortest strand of the dsRNA minus any overhangs that are present in the duplex. In addition to the duplex structure, a dsRNA may comprise one or more nucleoside overhangs. In one embodiment of the dsRNA, at least one strand comprises a 3′ overhang of at least 1 nucleoside. In another embodiment, at least one strand comprises a 3′ overhang of at least 2 linked nucleosides, e.g., 2, 3, 4, 5, 6, 7, 9, 10, 11, 12, 13, 14, or 15 linked nucleosides. In other embodiments, at least one strand of the dsRNA comprises a 5′ overhang of at least 1 nucleoside. In certain embodiments, at least one strand comprises a 5′ overhang of at least 2 linked nucleosides, e.g., 2, 3, 4, 5, 6, 7, 9, 10, 11, 12, 13, 14, or 15 linked nucleosides. In still other embodiments, both the 3′ and the 5′ end of one strand of the dsRNA comprise an overhang of at least 1 nucleoside.

Linkers or linking groups also refer to a connection between two atoms that links one chemical group or segment of interest to another chemical group or segment of interest via one or more covalent bonds. Conjugate moieties can be attached to the dsRNA directly or through a linking moiety (e.g. linker or tether). Linkers serve to covalently connect a third region, e.g. a conjugate moiety to a dsRNA (e.g. the termini of region A or C). In some embodiments of the invention the conjugate or dsRNA conjugate of the invention may optionally, comprise a linker region which is positioned between the dsRNA and the conjugate moiety. In some embodiments, the linker between the conjugate and dsRNA is biocleavable. Phosphodiester containing biocleavable linkers are described in more detail in WO 2014/076195 (herein incorporated by reference).

As used herein, the term “nucleoside overhang” refers to at least one unpaired nucleobase that protrudes from the duplex structure of a dsRNA or siRNA. For example, when a 3′-end of one strand of a dsRNA extends beyond the 5′-end of the other strand, or vice versa, there is a nucleoside overhang. A dsRNA can comprise an overhang of at least one nucleoside; alternatively, the overhang can comprise at least two nucleosides, at least three nucleosides, at least four nucleosides, at least five nucleosides or more. A nucleoside overhang can comprise or consist of an alternative nucleoside, including a deoxynucleotide/nucleoside. The overhang(s) can be on the sense strand, the antisense strand, or any combination thereof. Furthermore, the nucleoside(s) of an overhang can be present on the 5′-end, 3′-end or both ends of either an antisense or sense strand of a dsRNA. In certain embodiments, the overhang includes a self-complementary portion such that the overhang is capable of forming a hairpin structure that is stable under physiological conditions.

The terms “a” and “an” refer to one or more.

### EXAMPLES

### General Methods and Sequence Listings

### Experimental Model and Subject Details

Study population (Patients with NASH)

The Cambridge cohort consisted of 58 consecutive NAFLD patients (NAFL: 19; NASH FO-2: 24; NASH F3-4: 15) recruited at NASH Service at the Cambridge University Hospital. All the patients had clinical and biopsy-proven diagnosis of NAFLD (patients with alternate diagnoses and fatty liver from different etiologies were excluded), histology scored by a trained human pathologist according to the NASH CRN Scoring System (NAS1), and snap-frozen tissue for research purposes (Gene Expression by Next Generations Sequencing, see below). All the comparisons have been carried out against the NAFLD group. This study was approved by the local Ethics Committee; the principles of the Declaration of Helsinki were followed. All patients gave their informed consent for the use of clinical/omics data and samples for research purposes. For NASH/HCC patient information from single cell sequencing from UCLA, detail patient information was attached in Table 1.

TABLE 1
Analysis of single cell sequencing of liver nuclei from NASH/HCC patient

| p_val | avg_logF [symbol] | pct.1 | pct.2 | p_val_adj | cluster | gene | Chrm | Start | End | Strand | Name | Type |
|---|---|---|---|---|---|---|---|---|---|---|---|---|
| 5.58E−123 | 0.372807 | 0.523 | 0.344 | 3.25E−118 | Hep-1 | ENSG00000186480.12 | chr7 | 155297776 | 155310235 | + | INSIG1 | protein_coding |
| 1.36E−114 | 0.354331 | 0.443 | 0.273 | 7.91E−110 | Hep-1 | ENSG00000186951.16 | chr22 | 46150521 | 46243756 | + | PPARA | protein_coding |
| 1.03E−112 | 1.664505 | 0.837 | 0.152 | 5.99E−108 | Chol-2 | ENSG00000160789.19 | chr1 | 156082573 | 156140089 | + | LMNA | protein_coding |
| 7.13E−50 | 0.418587 | 0.555 | 0.297 | 4.15E−45 | Hep-6 | ENSG00000186951.16 | chr22 | 46150521 | 46243756 | + | PPARA | protein_coding |
| 2.95E−47 | 0.772837 | 0.32 | 0.15 | 1.72E−42 | LSEC-PP | ENSG00000160789.19 | chr1 | 156082573 | 156140089 | + | LMNA | protein_coding |
| 3.10E−42 | 0.439884 | 0.295 | 0.149 | 1.80E−37 | Chol-1 | ENSG00000160789.19 | chr1 | 156082573 | 156140089 | + | LMNA | protein_coding |
| 2.31E−38 | 0.396356 | 0.676 | 0.505 | 1.34E−33 | Hep-5 | ENSG00000115306.15 | chr2 | 54456285 | 54671445 | + | SPTBN1 | protein_coding |
| 5.34E−35 | 0.328918 | 0.603 | 0.37 | 3.11E−30 | Hep-6 | ENSG00000186480.12 | chr7 | 155297776 | 155310235 | + | INSIG1 | protein_coding |
| 4.27E−30 | 0.693709 | 0.415 | 0.101 | 2.49E−25 | Chol-2 | ENSG00000112033.13 | chr6 | 35342558 | 35428191 | + | PPARD | protein_coding |
| 2.78E−28 | 0.321256 | 0.555 | 0.401 | 1.62E−23 | Chol-1 | ENSG00000163513.17 | chr3 | 30606502 | 30694142 | + | TGFBR2 | protein_coding |
| 3.29E−28 | 0.557019 | 0.54 | 0.403 | 1.91E−23 | LSEC-PP | ENSG00000163513.17 | chr3 | 30606502 | 30694142 | + | TGFBR2 | protein_coding |
| 1.38E−27 | 0.329247 | 0.518 | 0.371 | 8.03E−23 | Hep-4 | ENSG00000186480.12 | chr7 | 155297776 | 155310235 | + | INSIG1 | protein_coding |
| 5.52E−27 | 0.315623 | 0.648 | 0.505 | 3.21E−22 | Hep-4 | ENSG00000115306.15 | chr2 | 54456285 | 54671445 | + | SPTBN1 | protein_coding |
| 2.28E−20 | 0.355195 | 0.678 | 0.508 | 1.33E−15 | Hep-7 | ENSG00000115306.15 | chr2 | 54456285 | 54671445 | + | SPTBN1 | protein_coding |
| 2.25E−18 | 0.309887 | 0.391 | 0.233 | 1.31E−13 | Hep-7 | ENSG00000198911.11 | chr22 | 41833079 | 41907308 | + | SREBF2 | protein_coding |
| 2.29E−17 | 0.430105 | 0.276 | 0.073 | 1.34E−12 | Chol-2 | ENSG00000072310.16 | chr17 | 17810399 | 17837002 | − | SREBF1 | protein_coding |
| 5.81E−16 | 0.573896 | 0.245 | 0.152 | 3.38E−11 | Stellate | ENSG00000160789.19 | chr1 | 156082573 | 156140089 | + | LMNA | protein_coding |
| 1.03E−15 | 0.3198 | 0.535 | 0.374 | 6.02E−11 | Hep-7 | ENSG00000186480.12 | chr7 | 155297776 | 155310235 | + | INSIG1 | protein_coding |
| 1.51E−14 | 0.274569 | 0.328 | 0.233 | 8.77E−10 | Chol-1 | ENSG00000198911.11 | chr22 | 41833079 | 41907308 | + | SREBF2 | protein_coding |
| 7.65E−13 | 0.260307 | 0.6 | 0.509 | 4.45E−08 | LSEC-PP | ENSG00000115306.15 | chr2 | 54456285 | 54671445 | + | SPTBN1 | protein_coding |
| 1.78E−11 | 0.701164 | 0.326 | 0.146 | 1.04E−06 | MAC-Fe | ENSG00000111275.12 | chr12 | 111766887 | 111817529 | + | ALDH2 | protein_coding |
| 1.95E−10 | 0.292599 | 0.222 | 0.182 | 1.13E−05 | LSEC-CV | ENSG00000132170.19 | chr3 | 12287368 | 12434356 | + | PPARG | protein_coding |
| 9.98E−10 | 0.302748 | 0.127 | 0.057 | 5.81E−05 | Hep-9 | ENSG00000185591.9 | chr12 | 53380176 | 53416446 | + | SP1 | protein_coding |
| 1.27E−08 | 0.261584 | 0.425 | 0.406 | 0.00073911 | LSEC-CV | ENSG00000163513.17 | chr3 | 30606502 | 30694142 | + | TGFBR2 | protein_coding |
| 3.71E−08 | 0.388648 | 0.463 | 0.236 | 0.00216064 | Chol-2 | ENSG00000198911.11 | chr22 | 41833079 | 41907308 | + | SREBF2 | protein_coding |
| 7.16E−07 | 0.301858 | 0.219 | 0.183 | 0.04171039 | Kuppfer | ENSG00000132170.19 | chr3 | 12287368 | 12434356 | + | PPARG | protein_coding |

[symbol]

indicates data missing or illegible when filed

### Mouse Strains, HFD Feeding and DEN Injections

All animal experiments were performed according to the guidelines for the care and use of laboratory animals and were approved by the Institutional Biomedical Research Ethics Committee of The George Washington University for Biomedical Research. C57BL/6 mice were purchased from The Jackson Laboratories and were engineered. Both male and female mice were used in our study. To generate liver-specific deletion of Sptbn1 mice, Flox sites were inserted into the flanks of exon 24 to exon 26 of Sptbn1 gene locus, with Neo cassette. Neo cassettes were then removed by intercrossing with Flp mice. Sptbn1-Flox mice were then intercrossed with Albumin-Cre to generate liver-specific deletion of Sptbn1 mice. For high-fat diet (HFD) induced liver steatosis, 10 to 12-week-old male and female mice were fed with control diet or HFD (ENVIGO, Cat. TD.06414) for 12 weeks to 20 weeks. Blood glucose, TG, and cholesterol levels were measured by a glucometer, CardioChek PA analyzer, and PTS Panels Lipid Panel test strips. For DEN-induced liver cancer models, 25 mg/kg DEN was injected in 14-15 days old male and female mice. Tumor development was analyzed 6-10 months later. Liver and visceral adipose tissues were excised and weighed. Spleen, heart, brain, muscle, brown adipose tissues were also collected for further histological analysis.

### Generation of Cell Lines and Cell Culture

Sptbn1^{-/-}, Sptbn1^{+/-} and WT MEFs were generated from Sptbn1 intercrossed mice. Human liver cancer cell lines, Hep3B and HepG2, Huh7 as well as the mouse immortalized liver cell line, AML12, were purchased from ATCC and cultured in a complete culture medium, DMEM/F12 medium (Corning, Cat. 10-090-CV) supplemented with 1% Streptomycin-Penicillin and 10% FBS (Hyclone, Cat. SH30396.03). The human immortalized liver cell line THLE2, purchased from ATCC, was cultured in BEGM medium (Lonza/Clonetics Corporation, Cat. CC3170) supplemented with 1% Streptomycin-Penicillin, 10% FBS (Sigma-Aldrich, Cat. F2442), 40ug phosphor-ethanolamine (Sigma-Aldrich, Cat. P0503), and 3ug human recombinant EGF (Corning®, Cat. 354052) in addition to BPE (Bovine Pituitary Extract), hydrocortisone, hEGF, insulin, Triiodothyronine, transferrin, and retinoic acid from the BEGM culture medium kit (Lonza/Clonetics Corporation, Cat. CC3170). THLE2 cells were cultured using flasks or plates pre-coated with a mixture of 0.01 mg/ml fibronectin, 0.03 mg/ml bovine collagen type I and 0.01 mg/ml bovine serum albumin. For HepG2 cell line with stable knockout of Smad3 and/or Sptbn1 by CRISPR/Cas9, HepG2 cells plated on 6 well plates were transfected with Sptbn1/HDR knockout CRISPR plasmids (Santa Cruz, sc-401818, sc-401818-HDR) using Lipofectamine LTX (Invitrogen) and Opti-MEM medium (Invitrogen) on 3 wells each, according to the manufacturer's instruction. After 48 hours, Puromycin (5 μg/ml) was added into the media for the selection of stable knockout cells. The selective media were replaced every 2-3 days.

### Method Details

### Reagents

DMEM/F12 medium was obtained from Corning (Cat. 10-090-CV); Streptomycin-Penicillin (Corning, Cat. 30-002-C1), Fetal Bovine Serum was purchased from HyClone (Cat. SH30396.03), phosphor-ethanolamine (Cat. P0503), and cell line THEL2 was purchased from ATCC (ATCC Cat. CRL-2706).

Lipoprotein deficient serum from fecal calf was obtained from Sigma-Aldrich (Cat. S5394). BEGM culture medium kit was obtained from Lonza/Clonetics Corporation (Cat. CC3170). Human recombinant EGF was purchased from (Corning, Cat. 354052). Collagen-coated flasks for THLE2 cells were from Thermo Scientific (Cat. 132707). Targeting SPTBN1 by siRNA in mouse and human 3D Culture

For hydrodynamic injection of siRNA to mice tail, siSptbn1 and siCtrl were resuspended in Nuclease-free Water (Life Technologies) to 0.25 mM, and incubated with RNAiMAX transfection reagent (Invitrogen) by a 1:1 ratio at room temperature for 20 minutes. Then these complexes were diluted by TransIT®-QR hydrodynamic delivery solution (Mirus, Madison, WI) to 0.625 uM. Mice received 2.0 mL of diluted siSptbn1 (1.25 nmol) or siCtrl through hydrodynamic tail vein injection as previously described. The injection was repeated in 6 weeks with 2 weeks interval in between after 1 week of HFD fed. For human 3D perfused microphysical systems composed of primary human hepatocytes, hepatic Kupffer cells, and stellate cells, these cells were co-culture in medium enriched in fatty acids, sugars, and insulin for 2 weeks as a culture model of human NASH.

### Transfection and Cell Fractionation

Cells plated in 60 mm plate at day 0 were transfected with 3-5 μg of indicated plasmid mixes using Lipofectamine 3000 (Thermo Fisher Scientific, Cat. L3000015), according to manufacturer's instructions at day 1. For TGF-β treatment at day 2, cells were incubated in serum-free DMEM/F12 medium overnight. Then, TGF-β 1 (Sigma-Aldrich, Cat. T1654; R&D, Cat. 240-B) was added to a final concentration of 200 pM for 0.5, 2, 12, 24 or 48 hrs before harvesting the cells. For knockdown experiments, siRNA targeting Sptbn1 or Smad3 (Dharmacon) was transfected into HepG2 or Hep3B cells by Lipofectamine RNAiMAX (Thermo Fisher, Cat. 13778150) for 24 to 48 hrs.

For cell fractionation, cells were harvested and lysed with cytoplasmic lysis buffer and then nuclear lysis buffer following the manufacturer's instruction for the Nuclear/Cytosol Fractionation Kit (Biovision, Cat. K266-25).

### Immunoprecipitation and Immunoblotting

Cells with or without treatment were lysed with (20 mM Tris-HCL pH7.4, 150 mM NaCl, 1 mM EDTA, 1% Triton-X100, 1% sodium deoxycholate, 0.1% SDS) or NP-40 lysis buffer (50 mM Tris-HCl, pH 7.5, 0.15 M NaCl, 1% NP-40, 1 mM EDTA), freshly made with complete Protease Inhibitor and Phosphatase Inhibitor Cocktail (Sigma-Aldrich, Cat. 4906845001, and Cat. 11836170001), followed by SDS-PAGE and immunoblotting using the following antibodies: anti-SPTBN1 (In house, rabbit), anti-SREBP1 (Abcam, Cat. Ab191857), anti-Vinculin (Proteintech, Cat. 26520-1-AP) and anti-GAPDH (Santa Cruz, Cat. sc-32233), anti-Tubulin (Cell Signaling, Cat. 3873), and anti-Smad3 (Cell Signaling, Cat. 9513). For immunoprecipitation, each fractionated lysate or whole cell lysate was precleared for 30 mins to remove the non-specific binding, followed by immunoprecipitation for 3 hrs or overnight using Protein A/G Mix Magnetic Bead (MilliporeSigma™, Cat. LSKMAGAG02) mixed with anti-SPTBN1 (In house) (23) or rabbit IgG (Cell Signaling, Cat. 2729) as the control. Immunoprecipitated complexes (Magnetic Bead-lysate-antibody mix) were washed 4 times with wash buffer (10 mM EDTA-Tris, 150 mM NaCl, 20 mM MgCl2) and eluted with sample buffer followed by Western Blot analysis.

### Luciferase Assay

For transcriptional reporter assays, HepG2 or Hep3B cells were seeded at a density of 1×10⁴ cells per well in 24-well dishes. The next day, the cells were co-transfected with siRNA (control or siRNA targeting Sptbn1 and Smad3) cells and a luciferase reporter (containing a LDLR promoter or SCD promoter) using Lipofectamine 3000 (Thermo-Fisher, Cat. L3000015). For MEF cells deficient with SMAD3 (Smad3^{-/-}) or Sptbn1 (Sptbn1^{-/-}), Sptbn1^{+/-} and wild type, a luciferase reporter containing either wild type LDLR promoter region (pLDLR-luc, Addgene Cat. 14940), LDLR promoter region harboring a SREBP-unresponsive mutant SRE (pLDLR-luc MUT, Addgene Cat. 14945), or SCD1 promoter (pGL3-SCD1, a gift from Dr. Giovanni Sorrentino and Dr. Giannino Del Sal) were co-transfected into the cells with Renilla (Promega) using Lipofectamine 3000 (Thermo Fisher, Cat. L3000015). For rescue experiments, pLDLR-luc or pGL3-SCD1 were co-transfected with control plasmid or V5-Sptbn1 to Sptbn1^{-/-} MEF cells; or Flag-SMAD3 plasmids to Smad3^{-/-} MEF cells. After 24 hrs of transfection, the cells were treated with 200 pM TGF-β1 (R&D, Cat. 240-B) for 24 hrs following overnight serum starvation. In all luciferase assays, the expression plasmid Renilla (Promega) served as an internal control to correct for transfection efficiency. The cells were extracted using 100 μl of luciferase cell culture lysis reagent. Ten microliters of cell extract were used for measuring Renilla enzyme activity. Twenty microliters of cell extract were used for the luciferase assay using a dual luciferase assay kit according to the manufacturer's instructions (Promega, Cat. E1980). The luciferase activity was normalized to Renilla activity (AU) for each sample, and fold changes were calculated.

### Immunohistochemical and Immunofluorescence Analyses

For immunohistochemical analyses, mouse tissues were fixed in 10% formalin and embedded in paraffin in accordance with standard procedures. FFPE sections were stained with hematoxylin and eosin (H&E) to evaluate gross morphology and ballooning, with Oil-Red to determine triglyceride accumulation and Sirius red for fibrosis. Different cell types were also determined using Hepatocyte nuclear factor 4 (HNF4) (for hepatocytes), Desmin (for hepatic stellate cell), CD31 (for liver sinusoidal endothelial cells), F4/80 (for macrophages), CD142, ICAM1 and CK19 (for Cholangiocytes). Sections were labeled with antibodies specific to SPTBN1, SREBP1, and Smads (Cell Signaling Technology, 2899) antibodies. Diaminobenzidine was used as a chromogen.

For immunofluorescence analyses, cells were seeded onto glass bottom dishes at a density of 2×10⁴ cells per dish in DMEM/F12 supplemented with 10% FBS or 1% lipoprotein deficient serum (LPDS) for 12 hrs. Cells treated with Oleic acid, Staurosporine, TNF-α, with or without TGF-β for different time points were then fixed with 10% formalin for 15 mins followed by permeabilization using 0.1% saponin for 5 mins. Cells were then incubated with blocking solution (0.1% saponin, 10% Bovine serum albumin in PBS) for 1 hr at RT followed by incubation with primary antibody (1:200 anti-rabbit SREBP1, anti-rabbit-Sptbn1 and anti-rabbit SMAD3) overnight at 4° C. Next day, the cells were washed with PBS and incubated with secondary antibody (Anti-rabbit Alexa Fluor Plus 488, goat-anti-mouse Alexa Fluor 568, goat-anti-rabbit Alexa Fluor 488, Invitrogen) for 1 hr at RT. DAPI was used to counterstain nuclei. Images were taken using ×40 objectives on IX81 Olympus microscope and confocal microscope.

### RNA Extraction and RNA Integrity

Human RNA was isolated with the following protocol: biopsies were homogenized in STAT-60 (AMS biotechnology, CS-502) (1 ml) with a tissue homogenizer, mixed (vortexing) and centrifuged at 13,000 g/RT for 5 minutes; the supernatant was mixed with 200 μl chloroform (Sigma, Cat. 650471) and centrifuged (12,000 g/4° C./15 mins); the supernatant was then mixed with 500 ul isopropanol (Sigma, Cat. 33539) and centrifuged (10,000 g/4° C./10 minutes) to pellet RNA; the pellet was washed with ethanol (75%; 1 ml) and dried at RT; the RNA was re-suspended in DEPC treated water (Quality Biological, Cat. 351-068-131). All reagents, plastic ware, and supplies used were nuclease free, sterile, and of molecular biology grade. RNA purity (A260/A280>1.80) and concentration were determined using Nanodrop (Thermo Fisher Scientific, Delaware USA). RNA integrity was studied using the 2100 Bioanalyzer (RNA 6000 Nano Kit; Agilent, Santa Clara, California, USA): a RNA Integrity Number (RIN) of >8 was considered the lowest cut-off for sequencing. For mouse, RNA was extracted from either liver tissues or liver cells using RNeasy Plus Mini Kit (Qiagen, Cat. 74134). RNA quality and concentration were assessed using the Thermo Scientific Nanodrop 3300 spectrophotometer before RNA-sequencing (Novogene Corporation). For real-time PCR analysis, total RNA was extracted with the use of TRIzol reagent (Invitrogen, Cat. AM9738) according to the manufacturer's instructions. Reverse transcription of 1-2ug total RNA was performed by using a Super-Script III First-Strand Kit (Invitrogen). Each cDNA was amplified in triplicate with the iQ™ SYBR Green SuperMix PCR Kit (Bio-Rad Laboratories) for 40 cycles on a Bio-Rad system (iCycler Thermal Cycler). Primers for the Realtime PCR are listed in the following table.

### Whole-Transcriptome Amplification and RNA Sequencing

One microgram of RNA was used to generate barcoded sequencing libraries using Illumina TruSeq® Stranded mRNA Library Preparation Kit (Illumina) following manufacturer instructions. The sequencing libraries were normalized for concentration and combined into 96-plex pools and sequenced on 3 lanes of an Illumina HiSeq 4000 instrument at single-end 50 bp (SE50), yielding an average of 15 million reads/sample. Library preparation was performed by the Genomics and Transcriptomic Core at the Institute of Metabolic Science; the sequencing was performed at the Genomics Core, Cancer Research UK Cambridge Institute; both at the University of Cambridge.

### NGS Data Processing and Statistical Analysis

RNA sequencing data was aligned using hisat2 V2.1.0 2 to the human GRCh38 genome and Mus Musculus (GRCm38/mm10); the genes that passed the QC were counted using HTSeq2 (v 0.11.1) 3. The raw gene-level counts were then used for differential gene expression analysis using DESeq2 4. Gene Expression was normalized using Log2-Transformed Copies per Million (Log2CPM) and statistical significance (p<0.05) assessed by R using the Wald Test in DESeq2. The raw p values were then adjusted by the Benjamini-Hochberg procedure to control the False Discovery Rate (FDR)₅.

### Bioinformatics Functional Analyses

Differentially expressed genes within groups were studied using Ingenuity Pathway Analysis (IPA, Qiagen) imputing the whole transcriptome and filtering it in IPA for statistically significant (p<0.05) hits with a Log2FC less than −0.3785 or greater than 0.3785, and Log2CPM >0.5 for human and or FPKM >1 for mice. Upstream Regulators networks were generated and ranked in terms of significance of participating genes (p<0.05 for human) and activation status (Z-score). We considered “biologically relevant” only those genes that are statistically significant (p<0.05), with a Log2FC less than −0.3785 or greater than 0.3785, and enriched in ‘significantly modulated’ networks in the comparative analysis, and/or those genes with an FDR<0.05.

### Structure Modeling of Human SPTBN1 and SREBP1c

Three-dimensional structures of human SPTBN1 and SREBP1c are still unknown, thus in-silico structural modeling was performed through the homology-based fold recognition approach while using the Phyre2 (Protein Homology/analogY Recognition Engine V 2.0) web-based server (53). This approach uses multiple templates covering different query regions (with different identity hence, varying confidence) of an amino acid sequence to generate a three-dimensional model of a protein structure. At first, a template identification was carried out through the P-BLAST (Protein Basic Local Alignment Search Tool) against the RCSB Protein Data Bank (PDB), where several templates were found showing suitable sequence identity and coverage with SPTBN1 and SREBP1c to model their structures. The templates from the PDB and folds from the Phyre in-house fold library, having the highest sequence identity and coverage, were used to generate the models of the human SPTBN1 and SREBP1c (Table 2). The loop modeling was optimized while using the ModLoop (54) followed by model optimization using Swiss-PDB Viewer (55).

TABLE 2
Patient information of single cell sequencing of liver nuclei from NASH/HCC patient.

| | | | | | | | | | | Clinical dx | | | |
|---|---|---|---|---|---|---|---|---|---|---|---|---|---|
| | | | Weight* | Height* | | Dyslip- | | | | NASH in | Tumor | Differen- | |
| ID | | Sex | (kg) | (cm) | HTN | idemia | Statin? | CAD | T2D | hepatology? | size | tiation | Path |
| Sample 2 | Hispanic | F | 53.40 | 147.30 | Y | N | N | N | N | Y | 2.70 | well | Steatohepatitis with Mallory |
| (S2_hcc and | | | | | | | | | | | | | Denke bodies and ballooning, |
| S2_nash) | | | | | | | | | | | | | micro and macrovesicular |
| | | | | | | | | | | | | | steatotis (20%); trichrome with |
| | | | | | | | | | | | | | “no significant fibrosis |
| Sample 3 | Not | F | 56.30 | 157.50 | Y | N | N | N | Y | Y | 2.00 | moder- | “Across the lobules, there is no |
| (S3_hcc and | Hispanic | | | | | | | | | | | ately | significant inflammation. Focal |
| S3_nash) | | | | | | | | | | | | | areas with mild macrovesicular |
| | | | | | | | | | | | | | steatosis is seen (<10%), |
| | | | | | | | | | | | | | however, hepatocyte ballooning |
| | | | | | | | | | | | | | or Mallory-Denk bodies are not |
| | | | | | | | | | | | | | seen. Cholestasis is not present. |
| | | | | | | | | | | | | | Trichrome stain shows extensive |
| | | | | | | | | | | | | | bridging fibrosis but no definite |
| | | | | | | | | | | | | | nodularity (confirmed by |
| | | | | | | | | | | | | | reticulin stain).” |
| Sample 1 | Not | F | 76.40 | 164.00 | Y | Y | Y | Y | Y | N (only seen | 6.40 | poorly | “The core biopsy of the liver |
| (S1_hcc and | Hispanic | | | | | | | | | by once and | | | shows two foci of hepatocytes |
| S2_norm) | | | | | | | | | | surgery) | | | that exhibit large cell and small |
| | | | | | | | | | | | | | cell change/dysplasia with |
| | | | | | | | | | | | | | increased steatosis, but portal |
| | | | | | | | | | | | | | structures are still present in |
| | | | | | | | | | | | | | these areas. Otherwise, the |
| | | | | | | | | | | | | | biopsy exhibits relatively well- |
| | | | | | | | | | | | | | preserved hepatic architecture |
| | | | | | | | | | | | | | (confirmed by reticulin stain). |
| | | | | | | | | | | | | | There are minimal to mild |
| | | | | | | | | | | | | | inflammatory cell infiltrates in |
| | | | | | | | | | | | | | portal areas. Mild ductular |
| | | | | | | | | | | | | | reaction is seen. The native bile |
| | | | | | | | | | | | | | ducts appear unremarkable. |
| | | | | | | | | | | | | | There is patchy mild sinusoidal |
| | | | | | | | | | | | | | dilatation. Focal steatosis and |
| | | | | | | | | | | | | | scattered glycogenated nuclei |
| | | | | | | | | | | | | | are noted. No Mallory-Denk |
| | | | | | | | | | | | | | bodies are identified. Trichrome |
| | | | | | | | | | | | | | stain shows portal fibrosis in a |
| | | | | | | | | | | | | | few portal tracts. No bridging |
| | | | | | | | | | | | | | fibrosis or features of cirrhosis |
| | | | | | | | | | | | | | are demonstrated. PAS stain |
| | | | | | | | | | | | | | confirms the above findings. |
| | | | | | | | | | | | | | PASD stain highlights small |
| | | | | | | | | | | | | | intracytoplasmic hyaline globules |
| | | | | | | | | | | | | | in some of hepatocytes, |
| | | | | | | | | | | | | | suggestive of megamitochondria |
| | | | | | | | | | | | | | (immune-stain for alpha-1- |
| | | | | | | | | | | | | | antitrypsin pending). Iron stain |
| | | | | | | | | | | | | | reveals minimal iron deposition in |
| | | | | | | | | | | | | | rare hepatocytes.” |

Model optimization and energy minimization were also performed to find the most stable, lowest energy conformations of SPTBN1 and SREBP1c to avoid any consequential errors and high energy configurations which might lead to physical perturbations and instability of the structures. The Swiss-PDB Viewer was used in energy minimization and in optimizing the structures by changing coordinate geometries in such a way as to release internal constraints and reduce the total potential energy of the models.

### Structure-Based Molecular Docking

Further, after modeling SPTBN1 and SREBP1c structures, their binding pattern was explored by using the molecular docking approach. Structural coordinates of SPTBN1 fragments covering D50-T975, Q1132-T2155 and A2198-K2364, and SREBP1c fragments covering Q295-K374 and P546-S705 were taken from our modelled structures. The docking was performed using the ClusPro 2.0 webserver (38). First, the docking results were screened for higher binding affinity, and then top docked conformations were selected and further analyzed using PyMOL and LigPlot+ for their possible interactions.

### Statistics

Differences between 2 groups were evaluated using 2-tailed Student's t-tests using GraphPad Prism. Pairwise comparison was performed for body weight gain measured weekly. For multiple comparisons, One-Way ANOVA with post-hoc Bonferroni's test was used. In vitro experiments were performed 2-4 times. All luciferase experiments were performed at least 3 times in triplicate. Results are expressed as mean±SEM unless otherwise indicated. For all statistical analyses, p<0.05 was considered statistically significant.

### Various Human and Mouse Sequence Listings

Homo sapiens spectrin beta, non-erythrocytic 1
(SPTBN1), transcript variant 1, mRNA
NCBI Reference Sequence: NM_003128.3
(SEQ ID No. 1)
atgacgaccacagtagccacagactatgacaacattgaga
tccagcagcagtacagtgatgtcaacaaccgctgggatgt
cgacgactgggacaatgagaacagctctgcgcggcttttt
gagcggtcccgcatcaaggctctggcagatgagcgtgaag
ccgtgcagaagaagaccttcaccaagtgggtcaattccca
ccttgcccgtgtgtcctgccggatcacagacctgtacact
gaccttcgagatggacggatgctcatcaagctgctggagg
tcctctctggagagaggctgcctaaacccaccaagggacg
aatgcgcatccactgcttagagaatgtggacaaggccctt
cagttcctgaaggagcagagagtccatcttgagaacatgg
ggtcccatgacatcgtggatggaaaccaccggctgaccct
tggcctcatctggaccatcatcctgcgcttccagatccag
gatatcagtgtggaaactgaagacaacaaagagaagaaat
ctgccaaggatgcattgctgttgtggtgccagatgaagac
agctgggtaccccaatgtcaacattcacaatttcaccact
agctggagggacggcatggccttcaatgcactgatacaca
aacaccggcctgacctgatagattttgacaaactaaagaa
atctaacgcacactacaacctgcagaatgcatttaatctg
gcagaacagcacctcggcctcactaaactgttggaccccg
aagacatcagcgtggaccatcctgatgagaagtccataat
cacttatgtggtgacttattaccactacttctctaagatg
aaggccttagctgttgaaggaaaacgaattggaaaggtgc
ttgacaatgctattgaaacagaaaaaatgattgaaaagta
tgaatcacttgcctctgaccttctggaatggattgaacaa
accatcatcattctgaacaatcgcaaatttgccaattcac
tggtcggggttcaacagcagcttcaggcattcaacactta
ccgcactgtggagaaaccacccaaatttactgagaagggg
aacttggaagtgctgctcttcaccattcagagcaagatga
gggccaacaaccagaaggtctacatgccccgggaggggaa
gctcatctctgacatcaacaaggcctgggaaagactggaa
aaagcggaacacgaaagagaactggctttgcggaatgagc
tcataagacaggagaaactggaacagctcgcccgcagatt
tgatcgcaaggcagctatgagggagacttggctgagcgaa
aaccagcgtctggtgtctcaggacaactttgggtttgacc
ttcctgcagttgaggccgccacaaaaaagcacgaggccat
tgagacagacattgccgcatacgaggagcgtgtgcaggct
gtggtagccgtggccagggagctcgaggccgagaattacc
acgacatcaagcgcatcacagcgaggaaggacaatgtcat
ccggctctgggaatacctactggaactgctcagggcccgg
agacagcggctcgagatgaacctggggctgcagaagatat
tccaggaaatgctctacattatggactggatggatgaaat
gaaggtgctagtattgtctcaagactatggcaaacactta
cttggtgtggaagacctgttacagaagcacaccctggttg
aagcagacattggcatccaggcagagcgggtgagaggtgt
caatgcctccgcccagaagttcgcaacagacggggaaggt
tacaagccctgtgacccccaggtgatccgagaccgcgtgg
cccacatggagttctgttatcaagagctttgccagctggc
ggctgagcgcagggcccgtctggaagagtcccgccgcctc
tggaagttcttctgggagatggcagaagaggaaggctgga
tacgggagaaggagaagatcctgtcctcggacgattacgg
gaaagacctgaccagcgtcatgcgcctgctcagcaagcac
cgggcgttcgaggacgagatgagcggccgcagtggccact
ttgagcaggccatcaaggaaggcgaagacatgatcgcgga
ggagcacttcgggtcggagaagatccgtgagaggatcatt
tacatccgggagcagtgggccaacctagagcagctctcgg
ccattcggaagaagcgcctggaggaggcctccctgctgca
ccagttccaggcagatgctgatgacattgatgcctggatg
ctggacatcctcaagattgtctccagcagcgacgtgggcc
acgatgagtattccacacagtctctggtcaagaaacacaa
ggacgtggcggaagagatcgccaattacaggcccaccctt
gacacgctgcacgaacaagccagcgccctcccccaggagc
atgccgagtctccagacgtgaggggcaggctgtcgggcat
cgaggagcggtataaggaggtggcagagctgacgcggctg
cggaagcaggcactccaggacactctggccctgtacaaga
tgttcagcgaggctgatgcctgtgagctctggatcgacga
gaaggagcagtggctcaacaacatgcagatcccagagaag
ctggaggatctggaggtcatccagcacagatttgagagcc
tagaaccagaaatgaacaaccaggcttcccgggttgcagt
ggtgaaccagattgcacgccagctgatgcacagcggccac
ccaagtgagaaggaaatcaaagcccagcaggacaaactca
acacaaggtggagccagttcagagaactggttgacaggaa
gaaggatgccctcctgtctgccctgagcatccagaactac
cacctcgagtgcaatgaaaccaaatcctggattogggaaa
agaccaaggtcatcgagtccacccaggacctgggcaatga
cctggctggcgtcatggccctgcagcgcaagctgaccggc
atggagcgggacttggtggccattgaggcaaagctgagtg
acctgcagaaggaggcggagaagctggagtccgagcaccc
cgaccaggcccaggccatcctgtctcggctggccgagatc
agcgacgtgtgggaggagatgaagaccaccctgaaaaacc
gagaggcctccctgggagaggccagcaagctgcagcagtt
cctacgggacttggacgacttccagtcctggctctctagg
acccagacagcgatcgcctcggaggacatgccaaacaccc
tgaccgaggctgagaagctgctcacgcagcacgagaacat
caagaacgagatcgacaactacgaggaggactaccagaag
atgagggacatgggcgagatggtcacccaggggcagaccg
atgcccagtacatgtttctgcggcagcggctgcaggccct
ggacactggatggaacgagctccacaagatgtgggagaac
agacaaaatctcctatcccagtcacatgcctaccagcagt
tcctcagagacacgaagcaagccgaagcctttcttaacaa
ccaggagtatgttctggctcacactgaaatgcctaccacc
ttggaaggagctgaagcagcaattaaaaagcaagaggact
tcatgaccaccatggacgccaatgaggagaagatcaatgc
tgtggtggagactggccggaggctggtgagcgatgggaac
atcaactcagatcgcatccaggagaaggtggactctattg
atgacagacataggaagaatcgtgagacagccagtgaact
tttgatgaggttgaaggacaacagggatctacagaaattc
ctgcaagattgtcaagagctgtctctctggatcaatgaga
agatgctcacagcccaggacatgtcttacgatgaagccag
aaatctgcacagtaaatggttgaagcatcaagcatttatg
gcagaacttgcatccaacaaagaatggcttgacaaaatcg
agaaggaaggaatgcagctcatttcagaaaagcctgagac
ggaagctgtggtgaaggagaaactcactggtttacataaa
atgtgggaagtccttgaatccactacccagacaaaggccc
agcggctctttgatgcaaacaaggccgaacttttcaccca
gagctgtgcagatctagacaaatggctgcacggcctggag
agtcagattcagtctgatgactatggcaaagacctgacca
gtgtcaatatcctgctgaaaaagcaacagatgctggagaa
tcagatggaagtgcggaagaaggagatcgaagagctccaa
agccaagcccaggccctgagtcaggaagggaagagcaccg
acgaggtagacagcaagcgcctcaccgtgcagaccaagtt
catggagttgctggagcccttgaacgagaggaagcataac
ctgctggcctccaaagagatccatcagttcaacagggatg
tggaggacgagatcttgtgggttggagagaggatgccttt
ggcaacttccacggatcatggccacaacctccagactgtg
cagctgttaataaagaaaaatcagaccctccagaaagaaa
tccaggggcaccagcctcgcattgacgacatctttgagag
gagccaaaacatcgtcactgacagcagcagcctcagcgct
gaggccatcagacagaggcttgccgacctgaagcagctgt
ggggtctcctcattgaggagacagagaaacgccacaggcg
gctggaggaggcgcacagggcccagcagtactactttgac
gctgctgaggccgaagcctggatgagcgagcaggagctgt
acatgatgtcagaggagaaggccaaggatgagcagagtgc
tgtctccatgttgaagaagcaccagatcttagaacaagct
gtggaggactatgcagagaccgtgcatcagctctccaaga
ccagccgggccctggtggccgacagccatcctgaaagtga
gcgcattagcatgcggcagtccaaagtggataaactgtac
gctggtctgaaagaccttgctgaagagagaagaggcaagc
tggatgagagacacaggttattccagctcaaccgggaggt
ggacgacctggagcagtggatcgctgagagggaggtggtc
gcagggtcccatgaactgggacaggactatgagcatgtca
cgatgttacaagaacgattccgggagtttgcccgagacac
cgggaacattgggcaggagcgcgtggacacggtcaatcac
ctggcagatgagctcatcaactctggacattcagatgccg
ccaccatcgctgaatggaaggatggcctcaatgaagcctg
ggccgacctcctggagctcattgacacaagaacacagatt
cttgccgcttcctatgaactgcacaagttttaccacgatg
ccaaggagatctttgggcgtatacaggacaaacacaagaa
actccctgaggagcttgggagagatcagaacacagtggag
accttacagagaatgcacactacatttgagcatgacatcc
aggctctgggcacacaggtgaggcagctgcaggaggatgc
agcccgcctccaggcggcctatgcgggtgacaaggccgac
gatatccagaagcgcgagaacgaggtcctggaagcctgga
agtccctcctggacgcctgtgagagccgcagggtgcggct
ggtggacacaggggacaagttccgcttcttcagcatggtg
cgcgacctcatgctctggatggaggatgtcatccggcaga
tcgaggcccaggagaagccaagggatgtatcatctgttga
actcttaatgaataatcatcaaggcatcaaagctgaaatt
gatgcacgtaatgacagtttcacaacctgcattgaacttg
ggaaatccctgttggcgagaaaacactatgcatctgagga
gatcaaggaaaaattactgcagttgacggaaaagaggaaa
gaaatgatcgacaagtgggaagaccgatgggaatggttaa
gactgattctggaggtccatcagttctcaagagacgccag
tgtggccgaggcctggctgcttggacaggagccgtaccta
tccagccgagagataggccagagcgtggacgaggtggaga
agctcatcaagcgccacgaggcatttgaaaagtctgcagc
aacctgggatgagaggttctctgccctggaaaggctgact
acattggagttactggaagtgcgcagacagcaagaggaag
aggagaggaagaggcggccgccttctcccgagccgagcac
gaaggtttcagaggaagccgagtcccagcagcagtgggat
acttcaaaaggagaacaagtttcccaaaacggtttgccag
ctgaacagggatctccacggatggcagaaacggtggacac
aagcgaaatggtcaacggcgctacagaacaaaggacgagc
tctaaagagtccagccccatcccctccccgacctctgate
gtaaagccaagactgccctcccagcccagagtgccgccac
cttaccagccagaacccaggagacaccttcggcccagatg
gaaggcttcctcaatcggaaacacgagtgggaggcccaca
ataagaaagcctcaagcaggtcctggcacaatgtttattg
tgtcataaataaccaagaaatgggtttctacaaagatgca
aagactgctgcttctggaattccctaccacagcgaggtcc
ctgtgagtttgaaagaagctgtctgcgaagtggcccttga
ttacaaaaagaagaaacacgtattcaagctaagactaaat
gatggcaatgagtacctcttccaagccaaagacgatgagg
aaatgaacacatggatccaggctatctcttccgccatctc
ctctgataaacacgaggtgtctgccagcacccagagcacg
ccagcatccagccgegcgcagaccctccccaccagcgtcg
tcaccatcaccagcgagtccagtcccggcaagcgggaaaa
ggacaaagagaaagacaaagagaagcggttcagccttttt
ggcaaaaagaaatga.
Homo sapiens spectrin beta, non-erythrocytic 1
(SPTBN1), transcript variant 2, mRNA
NCBI Reference Sequence: NM_178313.2
(SEQ ID No. 2)
ATGGAATTGCAGAGGACGTCTAGTATCTCCGGGCCGCTGT
CGCCGGCGTACACGGGGCAGGTGCCTTACAACTACAACCA
GCTGGAAGGCAGATTCAAGCAGCTGCAAGATGAGCGTGAA
GCCGTGCAGAAGAAGACCTTCACCAAGTGGGTCAATTCCC
ACCTTGCCCGTGTGTCCTGCCGGATCACAGACCTGTACAC
TGACCTTCGAGATGGACGGATGCTCATCAAGCTGCTGGAG
GTCCTCTCTGGAGAGAGGCTGCCTAAACCCACCAAGGGAC
GAATGCGCATCCACTGCTTAGAGAATGTGGACAAGGCCCT
TCAGTTCCTGAAGGAGCAGAGAGTCCATCTTGAGAACATG
GGGTCCCATGACATCGTGGATGGAAACCACCGGCTGACCC
TTGGCCTCATCTGGACCATCATCCTGCGCTTCCAGATCCA
GGATATCAGTGTGGAAACTGAAGACAACAAAGAGAAGAAA
TCTGCCAAGGATGCATTGCTGTTGTGGTGCCAGATGAAGA
CAGCTGGGTACCCCAATGTCAACATTCACAATTTCACCAC
TAGCTGGAGGGACGGCATGGCCTTCAATGCACTGATACAC
AAACACCGGCCTGACCTGATAGATTTTGACAAACTAAAGA
AATCTAACGCACACTACAACCTGCAGAATGCATTTAATCT
GGCAGAACAGCACCTCGGCCTCACTAAACTGTTGGACCCC
GAAGACATCAGCGTGGACCATCCTGATGAGAAGTCCATAA
TCACTTATGTGGTGACTTATTACCACTACTTCTCTAAGAT
GAAGGCCTTAGCTGTTGAAGGAAAACGAATTGGAAAGGTG
CTTGACAATGCTATTGAAACAGAAAAAATGATTGAAAAGT
ATGAATCACTTGCCTCTGACCTTCTGGAATGGATTGAACA
AACCATCATCATTCTGAACAATCGCAAATTTGCCAATTCA
CTGGTCGGGGTTCAACAGCAGCTTCAGGCATTCAACACTT
ACCGCACTGTGGAGAAACCACCCAAATTTACTGAGAAGGG
GAACTTGGAAGTGCTGCTCTTCACCATTCAGAGCAAGATG
AGGGCCAACAACCAGAAGGTCTACATGCCCCGGGAGGGGA
AGCTCATCTCTGACATCAACAAGGCCTGGGAAAGACTGGA
AAAAGCGGAACACGAAAGAGAACTGGCTTTGCGGAATGAG
CTCATAAGACAGGAGAAACTGGAACAGCTCGCCCGCAGAT
TTGATCGCAAGGCAGCTATGAGGGAGACTTGGCTGAGCGA
AAACCAGCGTCTGGTGTCTCAGGACAACTTTGGGTTTGAC
CTTCCTGCAGTTGAGGCCGCCACAAAAAAGCACGAGGCCA
TTGAGACAGACATTGCCGCATACGAGGAGCGTGTGCAGGC
TGTGGTAGCCGTGGCCAGGGAGCTCGAGGCCGAGAATTAC
CACGACATCAAGCGCATCACAGCGAGGAAGGACAATGTCA
TCCGGCTCTGGGAATACCTACTGGAACTGCTCAGGGCCCG
GAGACAGCGGCTCGAGATGAACCTGGGGCTGCAGAAGATA
TTCCAGGAAATGCTCTACATTATGGACTGGATGGATGAAA
TGAAGGTGCTAGTATTGTCTCAAGACTATGGCAAACACTT
ACTTGGTGTGGAAGACCTGTTACAGAAGCACACCCTGGTT
GAAGCAGACATTGGCATCCAGGCAGAGCGGGTGAGAGGTG
TCAATGCCTCCGCCCAGAAGTTCGCAACAGACGGGGAAGG
TTACAAGCCCTGTGACCCCCAGGTGATCCGAGACCGCGTG
GCCCACATGGAGTTCTGTTATCAAGAGCTTTGCCAGCTGG
CGGCTGAGCGCAGGGCCCGTCTGGAAGAGTCCCGCCGCCT
CTGGAAGTTCTTCTGGGAGATGGCAGAAGAGGAAGGCTGG
ATACGGGAGAAGGAGAAGATCCTGTCCTCGGACGATTACG
GGAAAGACCTGACCAGCGTCATGCGCCTGCTCAGCAAGCA
CCGGGCGTTCGAGGACGAGATGAGCGGCCGCAGTGGCCAC
TTTGAGCAGGCCATCAAGGAAGGCGAAGACATGATCGCGG
AGGAGCACTTCGGGTCGGAGAAGATCCGTGAGAGGATCAT
TTACATCCGGGAGCAGTGGGCCAACCTAGAGCAGCTCTCG
GCCATTCGGAAGAAGCGCCTGGAGGAGGCCTCCCTGCTGC
ACCAGTTCCAGGCAGATGCTGATGACATTGATGCCTGGAT
GCTGGACATCCTCAAGATTGTCTCCAGCAGCGACGTGGGC
CACGATGAGTATTCCACACAGTCTCTGGTCAAGAAACACA
AGGACGTGGCGGAAGAGATCGCCAATTACAGGCCCACCCT
TGACACGCTGCACGAACAAGCCAGCGCCCTCCCCCAGGAG
CATGCCGAGTCTCCAGACGTGAGGGGCAGGCTGTCGGGCA
TCGAGGAGCGGTATAAGGAGGTGGCAGAGCTGACGCGGCT
GCGGAAGCAGGCACTCCAGGACACTCTGGCCCTGTACAAG
ATGTTCAGCGAGGCTGATGCCTGTGAGCTCTGGATCGACG
AGAAGGAGCAGTGGCTCAACAACATGCAGATCCCAGAGAA
GCTGGAGGATCTGGAGGTCATCCAGCACAGATTTGAGAGC
CTAGAACCAGAAATGAACAACCAGGCTTCCCGGGTTGCAG
TGGTGAACCAGATTGCACGCCAGCTGATGCACAGCGGCCA
CCCAAGTGAGAAGGAAATCAAAGCCCAGCAGGACAAACTC
AACACAAGGTGGAGCCAGTTCAGAGAACTGGTTGACAGGA
AGAAGGATGCCCTCCTGTCTGCCCTGAGCATCCAGAACTA
CCACCTCGAGTGCAATGAAACCAAATCCTGGATTCGGGAA
AAGACCAAGGTCATCGAGTCCACCCAGGACCTGGGCAATG
ACCTGGCTGGCGTCATGGCCCTGCAGCGCAAGCTGACCGG
CATGGAGCGGGACTTGGTGGCCATTGAGGCAAAGCTGAGT
GACCTGCAGAAGGAGGCGGAGAAGCTGGAGTCCGAGCACC
CCGACCAGGCCCAGGCCATCCTGTCTCGGCTGGCCGAGAT
CAGCGACGTGTGGGAGGAGATGAAGACCACCCTGAAAAAC
CGAGAGGCCTCCCTGGGAGAGGCCAGCAAGCTGCAGCAGT
TCCTACGGGACTTGGACGACTTCCAGTCCTGGCTCTCTAG
GACCCAGACAGCGATCGCCTCGGAGGACATGCCAAACACC
CTGACCGAGGCTGAGAAGCTGCTCACGCAGCACGAGAACA
TCAAGAACGAGATCGACAACTACGAGGAGGACTACCAGAA
GATGAGGGACATGGGCGAGATGGTCACCCAGGGGCAGACC
GATGCCCAGTACATGTTTCTGCGGCAGCGGCTGCAGGCCC
TGGACACTGGATGGAACGAGCTCCACAAGATGTGGGAGAA
CAGACAAAATCTCCTATCCCAGTCACATGCCTACCAGCAG
TTCCTCAGAGACACGAAGCAAGCCGAAGCCTTTCTTAACA
ACCAGGAGTATGTTCTGGCTCACACTGAAATGCCTACCAC
CTTGGAAGGAGCTGAAGCAGCAATTAAAAAGCAAGAGGAC
TTCATGACCACCATGGACGCCAATGAGGAGAAGATCAATG
CTGTGGTGGAGACTGGCCGGAGGCTGGTGAGCGATGGGAA
CATCAACTCAGATCGCATCCAGGAGAAGGTGGACTCTATT
GATGACAGACATAGGAAGAATCGTGAGACAGCCAGTGAAC
TTTTGATGAGGTTGAAGGACAACAGGGATCTACAGAAATT
CCTGCAAGATTGTCAAGAGCTGTCTCTCTGGATCAATGAG
AAGATGCTCACAGCCCAGGACATGTCTTACGATGAAGCCA
GAAATCTGCACAGTAAATGGTTGAAGCATCAAGCATTTAT
GGCAGAACTTGCATCCAACAAAGAATGGCTTGACAAAATC
GAGAAGGAAGGAATGCAGCTCATTTCAGAAAAGCCTGAGA
CGGAAGCTGTGGTGAAGGAGAAACTCACTGGTTTACATAA
AATGTGGGAAGTCCTTGAATCCACTACCCAGACAAAGGCC
CAGCGGCTCTTTGATGCAAACAAGGCCGAACTTTTCACCC
AGAGCTGTGCAGATCTAGACAAATGGCTGCACGGCCTGGA
GAGTCAGATTCAGTCTGATGACTATGGCAAAGACCTGACC
AGTGTCAATATCCTGCTGAAAAAGCAACAGATGCTGGAGA
ATCAGATGGAAGTGCGGAAGAAGGAGATCGAAGAGCTCCA
AAGCCAAGCCCAGGCCCTGAGTCAGGAAGGGAAGAGCACC
GACGAGGTAGACAGCAAGCGCCTCACCGTGCAGACCAAGT
TCATGGAGTTGCTGGAGCCCTTGAACGAGAGGAAGCATAA
CCTGCTGGCCTCCAAAGAGATCCATCAGTTCAACAGGGAT
GTGGAGGACGAGATCTTGTGGGTTGGAGAGAGGATGCCTT
TGGCAACTTCCACGGATCATGGCCACAACCTCCAGACTGT
GCAGCTGTTAATAAAGAAAAATCAGACCCTCCAGAAAGAA
ATCCAGGGGCACCAGCCTCGCATTGACGACATCTTTGAGA
GGAGCCAAAACATCGTCACTGACAGCAGCAGCCTCAGCGC
TGAGGCCATCAGACAGAGGCTTGCCGACCTGAAGCAGCTG
TGGGGTCTCCTCATTGAGGAGACAGAGAAACGCCACAGGC
GGCTGGAGGAGGCGCACAGGGCCCAGCAGTACTACTTTGA
CGCTGCTGAGGCCGAAGCCTGGATGAGCGAGCAGGAGCTG
TACATGATGTCAGAGGAGAAGGCCAAGGATGAGCAGAGTG
CTGTCTCCATGTTGAAGAAGCACCAGATCTTAGAACAAGC
TGTGGAGGACTATGCAGAGACCGTGCATCAGCTCTCCAAG
ACCAGCCGGGCCCTGGTGGCCGACAGCCATCCTGAAAGTG
AGCGCATTAGCATGCGGCAGTCCAAAGTGGATAAACTGTA
CGCTGGTCTGAAAGACCTTGCTGAAGAGAGAAGAGGCAAG
CTGGATGAGAGACACAGGTTATTCCAGCTCAACCGGGAGG
TGGACGACCTGGAGCAGTGGATCGCTGAGAGGGAGGTGGT
CGCAGGGTCCCATGAACTGGGACAGGACTATGAGCATGTC
ACGATGTTACAAGAACGATTCCGGGAGTTTGCCCGAGACA
CCGGGAACATTGGGCAGGAGCGCGTGGACACGGTCAATCA
CCTGGCAGATGAGCTCATCAACTCTGGACATTCAGATGCC
GCCACCATCGCTGAATGGAAGGATGGCCTCAATGAAGCCT
GGGCCGACCTCCTGGAGCTCATTGACACAAGAACACAGAT
TCTTGCCGCTTCCTATGAACTGCACAAGTTTTACCACGAT
GCCAAGGAGATCTTTGGGCGTATACAGGACAAACACAAGA
AACTCCCTGAGGAGCTTGGGAGAGATCAGAACACAGTGGA
GACCTTACAGAGAATGCACACTACATTTGAGCATGACATC
CAGGCTCTGGGCACACAGGTGAGGCAGCTGCAGGAGGATG
CAGCCCGCCTCCAGGCGGCCTATGCGGGTGACAAGGCCGA
CGATATCCAGAAGCGCGAGAACGAGGTCCTGGAAGCCTGG
AAGTCCCTCCTGGACGCCTGTGAGAGCCGCAGGGTGCGGC
TGGTGGACACAGGGGACAAGTTCCGCTTCTTCAGCATGGT
GCGCGACCTCATGCTCTGGATGGAGGATGTCATCCGGCAG
ATCGAGGCCCAGGAGAAGCCAAGGGATGTATCATCTGTTG
AACTCTTAATGAATAATCATCAAGGCATCAAAGCTGAAAT
TGATGCACGTAATGACAGTTTCACAACCTGCATTGAACTT
GGGAAATCCCTGTTGGCGAGAAAACACTATGCATCTGAGG
AGATCAAGGAAAAATTACTGCAGTTGACGGAAAAGAGGAA
AGAAATGATCGACAAGTGGGAAGACCGATGGGAATGGTTA
AGACTGATTCTGGAGGTCCATCAGTTCTCAAGAGACGCCA
GTGTGGCCGAGGCCTGGCTGCTTGGACAGGAGCCGTACCT
ATCCAGCCGAGAGATAGGCCAGAGCGTGGACGAGGTGGAG
AAGCTCATCAAGCGCCACGAGGCATTTGAAAAGTCTGCAG
CAACCTGGGATGAGAGGTTCTCTGCCCTGGAAAGGCTGAC
TACATTGGAGTTACTGGAAGTGCGCAGACAGCAAGAGGAA
GAGGAGAGGAAGAGGCGGCCGCCTTCTCCCGAGCCGAGCA
CGAAGGTTTCAGAGGAAGCCGAGTCCCAGCAGCAGTGGGA
TACTTCAAAAGGAGAACAAGTTTCCCAAAACGGTTTGCCA
GCTGAACAGGGATCTCCACGGGTTAGTTACCGCTCTCAAA
CCTACCAAAACTACAAAAACTTTAATAGCAGACGGACAGC
CAGTGACCAGCCATGGTCTGGACTG.
Mus musculus spectrin beta, non-erythrocytic 1
(Sptbn1), transcript variant 1, mRNA
NCBI Reference Sequence: NM_175836.2
(SEQ ID No. 3)
atgacgaccacggtagccacagactatgacaacattgaga
tccagcagcagtacagtgatgtgaacaaccgctgggatgt
ggatgactgggacaatgagaacagctctgcaaggctcttt
gagcgctcccgcatcaaggccctggcagatgagcgtgaag
ctgtacagaagaagaccttcaccaagtgggtcaattccca
ccttgcgagagtgtcctgccgaatcacagacctgtacacg
gaccttcgagatggacggatgctcatcaagctactggagg
tcctctctggagagaggctgcctaaacccactaagggacg
gatgcggatccactgtctggagaatgtcgacaaggctctt
caattcctgaaagagcagagagtccatcttgagaacatgg
gctcccatgacattgtggatggaaaccaccggctgaccct
cggcctcatctggacaattattctgcgcttccagatccag
gatattagtgtggagactgaagataacaaagagaaaaagt
ctgctaaggatgcattgctgctgtggtgccagatgaagac
agctgggtaccccaatgtcaacattcacaatttcaccact
agctggagggatggcatggccttcaatgcactgatacata
aacatcggcctgacctgatagattttgataaactgaagaa
atctaatgcacactacaatctgcagaatgcatttaacctg
gcagagcagcaccttggcctcactaaactgttagaccctg
aagatatcagtgtggaccaccctgatgagaagtctatcat
cacatacgtggtgacttactaccactacttctccaagatg
aaggccttggctgtcgaaggaaagcgcattggaaaggtgc
ttgataatgctatagaaacagagaaaatgattgagaagta
cgagtcacttgcttctgaccttctggagtggattgaacaa
accatcatcatcctaaacaaccgcaaatttgctaattcac
tggttggggtccagcagcagctccaggcattcaacacgta
ccgcacagtggagaaaccacctaagtttactgagaagggg
aatttggaggtgctccttttcacaattcagagcaagatgc
gagcgaataatcagaaggtctacatgccccgcgaggggaa
gctcatctctgacatcaacaaggcctgggaaagactggaa
aaagcagaacatgagagagaactggctctgcggaatgagc
tcatacggcaggaaaaactggaacaactcgcccgaagatt
tgatcgcaaggcagctatgagggagacatggctgagtgaa
aaccagcgtcttgtgtctcaggacaactttggatttgacc
ttcccgctgttgaggctgctaccaaaaaacacgaggccat
tgagacagacatcgctgcatatgaagaacgagttcaggcc
gtggtggctgtggccagggaacttgaagccgagaactacc
atgacatcaagcgcatcacagcgaggaaggacaatgtcat
ccggctctgggaatacttgctggaactgctcagggccagg
aggcagcgtcttgagatgaacctgggattgcaaaagatat
tccaggaaatgctttatattatggactggatggatgaaat
gaaggtgctattgctgtctcaagactatggcaaacactta
cttggtgttgaagacctgttacagaagcatgccctggttg
aagcagacattgcaatccaagcagagcgtgtaagaggtgt
gaatgcctctgcccagaagtttgcaacagatggggaaggc
tacaagccatgtgacccccaggtaattcgagaccgtgttg
cccacatggagttctgctatcaagagctttgtcagctggc
tgccgagcgtagggctcgcctggaagagtcccgtcgcctc
tggaagttcttctgggagatggcagaagaggaaggctgga
tacgagagaaggagaagatcctgtcctctgatgattacgg
gaaagacttgaccagtgtcatgcgcctgctgagcaagcac
cgggcatttgaggatgagatgagtggccgtagtggccatt
ttgagcaggccattaaagaaggtgaagacatgattgcaga
ggaacactttggatcggaaaagatccgtgagagaatcatt
tatatccgggagcagtgggccaacctggaacagctctcag
ccattaggaagaagcgcctagaggaagcctcattactgca
ccagttccaggctgatgctgatgatattgatgcttggatg
ttagatatactcaagattgtctccagcaatgatgtgggcc
atgatgagtactccacgcagtctctggtcaagaagcataa
agatgtagcagaagagatcaccaactacaggcccactatt
gacacactgcatgagcaagccagtgcccttccacaagcac
atgcagagtctccagatgtgaagggccggctggcaggaat
tgaggagcgctgcaaggagatggcagagttaacacggcta
aggaagcaggctctgcaggacaccctggccctgtacaaga
tgttcagtgaggctgatgcctgtgagctctggattgacga
gaaggagcagtggctcaacaacatgcagatcccagagaag
ctggaggacctggaagtcatccagcacagatttgagagcc
tagaaccagaaatgaacaaccaggcttcccgggttgctgt
ggtgaaccagattgcacggcagctgatgcacaatggccac
cccagtgaaaaggaaatcagagctcagcaagacaaactca
acacgaggtggagtcagttcagagaactggtggacaggaa
aaaggatgctcttctgtctgccctgagcatccagaactac
cacctcgagtgcaatgaaaccaaatcctggatccgggaga
agaccaaggtcatcgagtctacccaagaccttggcaatga
cctggcaggtgtcatggccctgcagcgcaagctgactggc
atggaacgagacttggtagccattgaggcgaagctgagtg
acctgcagaaagaagctgagaagctggagtccgagcaccc
tgaccaggctcaagctatcctgtctcggctggccgagatc
agtgatgtgtgggaggaaatgaagacaaccctgaagaacc
gagaggcctccctgggagaggccagcaagctgcagcagtt
tctgcgggacttggacgacttccagtcttggctctccagg
acccagactgctatcgcctcagaggacatgcccaataccc
tcactgaggcagagaagcttctcacacagcacgagaatat
caaaaatgagatcgacaattatgaggaagactaccagaag
atgcgggacatgggcgagatggtcacccaggggcagactg
atgcccagtatatgtttctgcggcagcggctgcaggcctt
agacactggctggaatgagctccacaaaatgtgggagaac
aggcaaaacctcctctcccagtcccatgcctaccagcagt
tccttagggacaccaaacaagctgaagcttttcttaataa
ccaggagtatgttttggctcatactgaaatgcccaccacc
ctggaaggagctgaagcagccattaaaaagcaggaggact
tcatgaccaccatggatgccaacgaggagaagatcaatgc
tgttgtggagactggccgaagactggtgagcgatgggaac
atcaactccgaccgcatccaggagaaggtggactctattg
acgacagacacaggaagaatcgagaagcagccagtgaact
tctgatgaggttaaaggacaaccgtgatctacagaagttc
ctgcaagattgtcaagagctgtccctctggatcaatgaaa
agatgcttacagctcaagacatgtcttatgatgaagccag
aaatctgcacagtaaatggttaaagcatcaagcatttatg
gcggaacttgcatccaacaaagaatggcttgacaaaattg
agaaggaaggaatgcagcttatttcagaaaagccagaaac
agaagctgtggtaaaggaaaaactcactggtttacataaa
atgtgggaagtccttgaatccacaacccagaccaaggccc
agcggctctttgatgcaaataaggctgagcttttcacaca
aagctgcgcagatcttgacaaatggctacatggcctggag
agccagattcaatctgacgactatggcaaagaccttacca
gtgtcaatattcttctgaaaaagcaacagatgctggagaa
tcagatggaagttcggaagaaagagatcgaggaactgcag
agccaagcccaggcgctgagtcaggaggggaagagcacag
atgaggtggacagcaaacgccttactgtgcagaccaagtt
catggagcttctggagcccttgagtgagaggaagcataac
ctgttagcttccaaggagatccatcagttcaacagggatg
tggaggacgaaatcctatgggttggcgagaggatgccttt
ggcaacttccacagatcatggccataaccttcaaactgtg
cagctgttaataaagaaaaaccagaccctccagaaagaaa
tccagggacaccagcctcgtattgatgacatctttgagag
gagtcaaaacatcatcacagatagcagcagcctcaatgcc
gaggctatcaggcagaggctcgctgacctgaagcagctgt
gggggctcctcattgaggaaactgagaaacgccatagacg
gctggaggaggcacacaaggcgcagcagtactactttgat
gcagctgaagccgaggcatggatgagtgaacaggagttgt
acatgatgtctgaggaaaaggccaaggatgagcagagtgc
tgtctctatgttgaaaaagcaccagattttagagcaagct
gttgaggactatgcagagacagtacaccagctctccaaga
ctagccgggcgctggtggctgacagccatcccgaaagtga
gcgtattagcatgcggcagtcaaaggtcgacaagctgtat
gctggcctgaaggaccttgctgaggagaggagaggaaaac
ttgatgagaggcacaggctgttccagctcaacagagaggt
ggatgacctggaacagtggatcgctgagagggaagtggtc
gcaggctcccatgagttgggacaggactatgagcatgtca
cgatgttacaagaacggttccgagaatttgctcgagacac
aggaaacattgggcaggagcgtgtggatacagttaataac
atggcagatgaactcatcaactctggacattcagatgctg
ccaccattgctgagtggaaagatggtctcaatgaagcctg
ggctgacctcctggagctcattgacacaagaacacagatt
cttgctgcctcatatgaacttcataagttttaccatgatg
ccaaggagatctttggccgaatccaggacaaacacaagaa
actccctgaggagcttggaagagatcaaaacactgtggaa
actttacagagaatgcacaccacctttgagcacgacatcc
aagctctgggcactcaggtgaggcagctgcaggaggatgc
agctcgcctccaggcagcctatgcaggggacaaggctgat
gacatccagaagcgtgagaatgaggtcctggaagcctgga
agtccctgctggatgcttgtgagggtcgcagggtgcggct
ggtagacacaggagacaagttccgcttcttcagcatggtg
cgtgacctcatgctctggatggaagatgtcatccggcaga
tcgaggcccaggagaaaccacgggatgtgtcatctgttga
actgttaatgaataatcatcaaggtatcaaagctgaaatt
gatgctcgtaatgacagctttacagcctgcattgagcttg
ggaaatccctgctggcacggaaacactatgcttctgagga
gatcaaggaaaagttactgcagctgacagagaaaagaaaa
gaaatgattgacaagtgggaagaccggtgggagtggttaa
gactgattttggaggtccatcagttctcaagggatgccag
tgtggcagaggcttggctgcttggacaggaaccataccta
tccagccgtgaaattggccagagtgtagacgaagtggaga
agcttattaagcgccatgaggcgtttgaaaagtctgcagc
gacctgggatgagagattctctgctctggaaaggctgaca
acgttggagctactggaagtgcgcagacagcaagaggaag
aagaaagaaagaggcggccaccttctccggacccaaacac
gaaggtttcagaggaggctgagtcccagcaatgggatact
tcaaaaggagaccaagtttcccagaatggtttgccggctg
agcagggatctccacggatggcaggaaccatggaaacgag
tgaaatggtcaacggtgctgctgagcagaggacaagctcc
aaagagtccagtcctgttccctctcccaccttggaccgaa
aggccaaatctgcacttccagcccagagtgctgccaccct
gccagccaggaccctggagacacccgctgcccagatggaa
ggcttcctcaatcggaagcatgagtgggaggcccacaata
agaaagcctcgagcaggtcctggcacaatgtatattgtgt
cataaataaccaagaaatgggcttctataaagatgccaag
agtgctgcttctggcatcccctaccacagtgaggtccctg
tgagtttgaaagaggccatctgcgaagtggcccttgatta
caaaaagaagaagcacgtgttcaagctaagactaagtgat
ggaaacgagtacctcttccaagccaaagatgatgaggaaa
tgaacacatggatccaggctatctcctctgccatctcctc
tgacaaacacgacacatctgccagcacccagagtacgcca
gcatccagtcgggcgcagaccttacccaccagcgtcgtca
ccatcaccagcgagtccagtcctggcaagagggagaagga
taaagagaaagacaaagagaagaggttcagccttttcggc
aagaagaagtga.
Mus musculus spectrin beta, non-erythrocytic 1
(Sptbn1), transcript variant 2, mRNA
NCBI Reference Sequence: NM_009260.2
(SEQ ID No. 4)
ATGGAGTTGCAGAGGACATCCAGCATTTCAGGGCCGCTGT
CGCCGGCCTACACCGGGCAGGTGCCTTACAACTACAACCA
ACTGGAAGGAAGATTCAAACAGCTCCAAGATGAGCGTGAA
GCTGTACAGAAGAAGACCTTCACCAAGTGGGTCAATTCCC
ACCTTGCGAGAGTGTCCTGCCGAATCACAGACCTGTACAC
GGACCTTCGAGATGGACGGATGCTCATCAAGCTACTGGAG
GTCCTCTCTGGAGAGAGGCTGCCTAAACCCACTAAGGGAC
GGATGCGGATCCACTGTCTGGAGAATGTCGACAAGGCTCT
TCAATTCCTGAAAGAGCAGAGAGTCCATCTTGAGAACATG
GGCTCCCATGACATTGTGGATGGAAACCACCGGCTGACCC
TCGGCCTCATCTGGACAATTATTCTGCGCTTCCAGATCCA
GGATATTAGTGTGGAGACTGAAGATAACAAAGAGAAAAAG
TCTGCTAAGGATGCATTGCTGCTGTGGTGCCAGATGAAGA
CAGCTGGGTACCCCAATGTCAACATTCACAATTTCACCAC
TAGCTGGAGGGATGGCATGGCCTTCAATGCACTGATACAT
AAACATCGGCCTGACCTGATAGATTTTGATAAACTGAAGA
AATCTAATGCACACTACAATCTGCAGAATGCATTTAACCT
GGCAGAGCAGCACCTTGGCCTCACTAAACTGTTAGACCCT
GAAGATATCAGTGTGGACCACCCTGATGAGAAGTCTATCA
TCACATACGTGGTGACTTACTACCACTACTTCTCCAAGAT
GAAGGCCTTGGCTGTCGAAGGAAAGCGCATTGGAAAGGTG
CTTGATAATGCTATAGAAACAGAGAAAATGATTGAGAAGT
ACGAGTCACTTGCTTCTGACCTTCTGGAGTGGATTGAACA
AACCATCATCATCCTAAACAACCGCAAATTTGCTAATTCA
CTGGTTGGGGTCCAGCAGCAGCTCCAGGCATTCAACACGT
ACCGCACAGTGGAGAAACCACCTAAGTTTACTGAGAAGGG
GAATTTGGAGGTGCTCCTTTTCACAATTCAGAGCAAGATG
CGAGCGAATAATCAGAAGGTCTACATGCCCCGCGAGGGGA
AGCTCATCTCTGACATCAACAAGGCCTGGGAAAGACTGGA
AAAAGCAGAACATGAGAGAGAACTGGCTCTGCGGAATGAG
CTCATACGGCAGGAAAAACTGGAACAACTCGCCCGAAGAT
TTGATCGCAAGGCAGCTATGAGGGAGACATGGCTGAGTGA
AAACCAGCGTCTTGTGTCTCAGGACAACTTTGGATTTGAC
CTTCCCGCTGTTGAGGCTGCTACCAAAAAACACGAGGCCA
TTGAGACAGACATCGCTGCATATGAAGAACGAGTTCAGGC
CGTGGTGGCTGTGGCCAGGGAACTTGAAGCCGAGAACTAC
CATGACATCAAGCGCATCACAGCGAGGAAGGACAATGTCA
TCCGGCTCTGGGAATACTTGCTGGAACTGCTCAGGGCCAG
GAGGCAGCGTCTTGAGATGAACCTGGGATTGCAAAAGATA
TTCCAGGAAATGCTTTATATTATGGACTGGATGGATGAAA
TGAAGGTGCTATTGCTGTCTCAAGACTATGGCAAACACTT
ACTTGGTGTTGAAGACCTGTTACAGAAGCATGCCCTGGTT
GAAGCAGACATTGCAATCCAAGCAGAGCGTGTAAGAGGTG
TGAATGCCTCTGCCCAGAAGTTTGCAACAGATGGGGAAGG
CTACAAGCCATGTGACCCCCAGGTAATTCGAGACCGTGTT
GCCCACATGGAGTTCTGCTATCAAGAGCTTTGTCAGCTGG
CTGCCGAGCGTAGGGCTCGCCTGGAAGAGTCCCGTCGCCT
CTGGAAGTTCTTCTGGGAGATGGCAGAAGAGGAAGGCTGG
ATACGAGAGAAGGAGAAGATCCTGTCCTCTGATGATTACG
GGAAAGACTTGACCAGTGTCATGCGCCTGCTGAGCAAGCA
CCGGGCATTTGAGGATGAGATGAGTGGCCGTAGTGGCCAT
TTTGAGCAGGCCATTAAAGAAGGTGAAGACATGATTGCAG
AGGAACACTTTGGATCGGAAAAGATCCGTGAGAGAATCAT
TTATATCCGGGAGCAGTGGGCCAACCTGGAACAGCTCTCA
GCCATTAGGAAGAAGCGCCTAGAGGAAGCCTCATTACTGC
ACCAGTTCCAGGCTGATGCTGATGATATTGATGCTTGGAT
GTTAGATATACTCAAGATTGTCTCCAGCAATGATGTGGGC
CATGATGAGTACTCCACGCAGTCTCTGGTCAAGAAGCATA
AAGATGTAGCAGAAGAGATCACCAACTACAGGCCCACTAT
TGACACACTGCATGAGCAAGCCAGTGCCCTTCCACAAGCA
CATGCAGAGTCTCCAGATGTGAAGGGCCGGCTGGCAGGAA
TTGAGGAGCGCTGCAAGGAGATGGCAGAGTTAACACGGCT
AAGGAAGCAGGCTCTGCAGGACACCCTGGCCCTGTACAAG
ATGTTCAGTGAGGCTGATGCCTGTGAGCTCTGGATTGACG
AGAAGGAGCAGTGGCTCAACAACATGCAGATCCCAGAGAA
GCTGGAGGACCTGGAAGTCATCCAGCACAGATTTGAGAGC
CTAGAACCAGAAATGAACAACCAGGCTTCCCGGGTTGCTG
TGGTGAACCAGATTGCACGGCAGCTGATGCACAATGGCCA
CCCCAGTGAAAAGGAAATCAGAGCTCAGCAAGACAAACTC
AACACGAGGTGGAGTCAGTTCAGAGAACTGGTGGACAGGA
AAAAGGATGCTCTTCTGTCTGCCCTGAGCATCCAGAACTA
CCACCTCGAGTGCAATGAAACCAAATCCTGGATCCGGGAG
AAGACCAAGGTCATCGAGTCTACCCAAGACCTTGGCAATG
ACCTGGCAGGTGTCATGGCCCTGCAGCGCAAGCTGACTGG
CATGGAACGAGACTTGGTAGCCATTGAGGCGAAGCTGAGT
GACCTGCAGAAAGAAGCTGAGAAGCTGGAGTCCGAGCACC
CTGACCAGGCTCAAGCTATCCTGTCTCGGCTGGCCGAGAT
CAGTGATGTGTGGGAGGAAATGAAGACAACCCTGAAGAAC
CGAGAGGCCTCCCTGGGAGAGGCCAGCAAGCTGCAGCAGT
TTCTGCGGGACTTGGACGACTTCCAGTCTTGGCTCTCCAG
GACCCAGACTGCTATCGCCTCAGAGGACATGCCCAATACC
CTCACTGAGGCAGAGAAGCTTCTCACACAGCACGAGAATA
TCAAAAATGAGATCGACAATTATGAGGAAGACTACCAGAA
GATGCGGGACATGGGCGAGATGGTCACCCAGGGGCAGACT
GATGCCCAGTATATGTTTCTGCGGCAGCGGCTGCAGGCCT
TAGACACTGGCTGGAATGAGCTCCACAAAATGTGGGAGAA
CAGGCAAAACCTCCTCTCCCAGTCCCATGCCTACCAGCAG
TTCCTTAGGGACACCAAACAAGCTGAAGCTTTTCTTAATA
ACCAGGAGTATGTTTTGGCTCATACTGAAATGCCCACCAC
CCTGGAAGGAGCTGAAGCAGCCATTAAAAAGCAGGAGGAC
TTCATGACCACCATGGATGCCAACGAGGAGAAGATCAATG
CTGTTGTGGAGACTGGCCGAAGACTGGTGAGCGATGGGAA
CATCAACTCCGACCGCATCCAGGAGAAGGTGGACTCTATT
GACGACAGACACAGGAAGAATCGAGAAGCAGCCAGTGAAC
TTCTGATGAGGTTAAAGGACAACCGTGATCTACAGAAGTT
CCTGCAAGATTGTCAAGAGCTGTCCCTCTGGATCAATGAA
AAGATGCTTACAGCTCAAGACATGTCTTATGATGAAGCCA
GAAATCTGCACAGTAAATGGTTAAAGCATCAAGCATTTAT
GGCGGAACTTGCATCCAACAAAGAATGGCTTGACAAAATT
GAGAAGGAAGGAATGCAGCTTATTTCAGAAAAGCCAGAAA
CAGAAGCTGTGGTAAAGGAAAAACTCACTGGTTTACATAA
AATGTGGGAAGTCCTTGAATCCACAACCCAGACCAAGGCC
CAGCGGCTCTTTGATGCAAATAAGGCTGAGCTTTTCACAC
AAAGCTGCGCAGATCTTGACAAATGGCTACATGGCCTGGA
GAGCCAGATTCAATCTGACGACTATGGCAAAGACCTTACC
AGTGTCAATATTCTTCTGAAAAAGCAACAGATGCTGGAGA
ATCAGATGGAAGTTCGGAAGAAAGAGATCGAGGAACTGCA
GAGCCAAGCCCAGGCGCTGAGTCAGGAGGGGAAGAGCACA
GATGAGGTGGACAGCAAACGCCTTACTGTGCAGACCAAGT
TCATGGAGCTTCTGGAGCCCTTGAGTGAGAGGAAGCATAA
CCTGTTAGCTTCCAAGGAGATCCATCAGTTCAACAGGGAT
GTGGAGGACGAAATCCTATGGGTTGGCGAGAGGATGCCTT
TGGCAACTTCCACAGATCATGGCCATAACCTTCAAACTGT
GCAGCTGTTAATAAAGAAAAACCAGACCCTCCAGAAAGAA
ATCCAGGGACACCAGCCTCGTATTGATGACATCTTTGAGA
GGAGTCAAAACATCATCACAGATAGCAGCAGCCTCAATGC
CGAGGCTATCAGGCAGAGGCTCGCTGACCTGAAGCAGCTG
TGGGGGCTCCTCATTGAGGAAACTGAGAAACGCCATAGAC
GGCTGGAGGAGGCACACAAGGCGCAGCAGTACTACTTTGA
TGCAGCTGAAGCCGAGGCATGGATGAGTGAACAGGAGTTG
TACATGATGTCTGAGGAAAAGGCCAAGGATGAGCAGAGTG
CTGTCTCTATGTTGAAAAAGCACCAGATTTTAGAGCAAGC
TGTTGAGGACTATGCAGAGACAGTACACCAGCTCTCCAAG
ACTAGCCGGGCGCTGGTGGCTGACAGCCATCCCGAAAGTG
AGCGTATTAGCATGCGGCAGTCAAAGGTCGACAAGCTGTA
TGCTGGCCTGAAGGACCTTGCTGAGGAGAGGAGAGGAAAA
CTTGATGAGAGGCACAGGCTGTTCCAGCTCAACAGAGAGG
TGGATGACCTGGAACAGTGGATCGCTGAGAGGGAAGTGGT
CGCAGGCTCCCATGAGTTGGGACAGGACTATGAGCATGTC
ACGATGTTACAAGAACGGTTCCGAGAATTTGCTCGAGACA
CAGGAAACATTGGGCAGGAGCGTGTGGATACAGTTAATAA
CATGGCAGATGAACTCATCAACTCTGGACATTCAGATGCT
GCCACCATTGCTGAGTGGAAAGATGGTCTCAATGAAGCCT
GGGCTGACCTCCTGGAGCTCATTGACACAAGAACACAGAT
TCTTGCTGCCTCATATGAACTTCATAAGTTTTACCATGAT
GCCAAGGAGATCTTTGGCCGAATCCAGGACAAACACAAGA
AACTCCCTGAGGAGCTTGGAAGAGATCAAAACACTGTGGA
AACTTTACAGAGAATGCACACCACCTTTGAGCACGACATC
CAAGCTCTGGGCACTCAGGTGAGGCAGCTGCAGGAGGATG
CAGCTCGCCTCCAGGCAGCCTATGCAGGGGACAAGGCTGA
TGACATCCAGAAGCGTGAGAATGAGGTCCTGGAAGCCTGG
AAGTCCCTGCTGGATGCTTGTGAGGGTCGCAGGGTGCGGC
TGGTAGACACAGGAGACAAGTTCCGCTTCTTCAGCATGGT
GCGTGACCTCATGCTCTGGATGGAAGATGTCATCCGGCAG
ATCGAGGCCCAGGAGAAACCACGGGATGTGTCATCTGTTG
AACTGTTAATGAATAATCATCAAGGTATCAAAGCTGAAAT
TGATGCTCGTAATGACAGCTTTACAGCCTGCATTGAGCTT
GGGAAATCCCTGCTGGCACGGAAACACTATGCTTCTGAGG
AGATCAAGGAAAAGTTACTGCAGCTGACAGAGAAAAGAAA
AGAAATGATTGACAAGTGGGAAGACCGGTGGGAGTGGTTA
AGACTGATTTTGGAGGTCCATCAGTTCTCAAGGGATGCCA
GTGTGGCAGAGGCTTGGCTGCTTGGACAGGAACCATACCT
ATCCAGCCGTGAAATTGGCCAGAGTGTAGACGAAGTGGAG
AAGCTTATTAAGCGCCATGAGGCGTTTGAAAAGTCTGCAG
CGACCTGGGATGAGAGATTCTCTGCTCTGGAAAGGCTGAC
AACGTTGGAGCTACTGGAAGTGCGCAGACAGCAAGAGGAA
GAAGAAAGAAAGAGGCGGCCACCTTCTCCGGACCCAAACA
CGAAGGTTTCAGAGGAGGCTGAGTCCCAGCAATGGGATAC
TTCAAAAGGAGACCAAGTTTCCCAGAATGGTTTGCCGGCT
GAGCAGGGATCTCCACGGGTTAGTTACCGCTCTCAAACGT
ACCAAAACTACAAAAACTTTAATAGCAGACGGACAGCCAG
TGACCATTCATGGTCTGGAATG.

### Mouse SiRNA Targeting Sptbn1

Target Sequence:

Sense:
(SEQ ID No. 5)
CGAUGUUACAAGAACGGUUTT.
Antisense:
(SEQ ID NO. 6)
AACCGUUCUUGUAACAUCGTG.

Human siRNA Targeting

Target Sequence:
(SEQ ID No. 7)
CCUGAAAGUGAGCGCAUUA.
Target Sequence:
(SEQ ID No. 8)
CCGCAUACGAGGAGCGUGU.
Target Sequence:
(SEQ ID No. 9)
GGACAUGUCUUACGAUGAA.
Target Sequence:
(SEQ ID NO. 10)
GUGACAAGGCCGACGAUAU.

### EXAMPLE 1

Liver-Specific SPTBN1 Knockout Protects Mice from HFD-Induced NASH

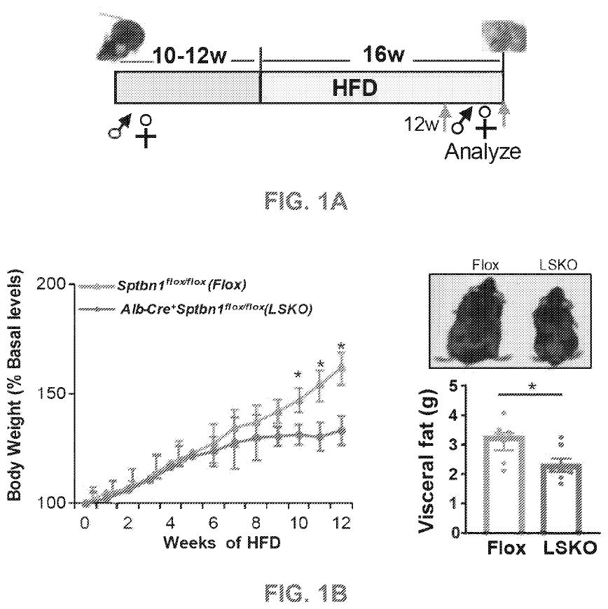

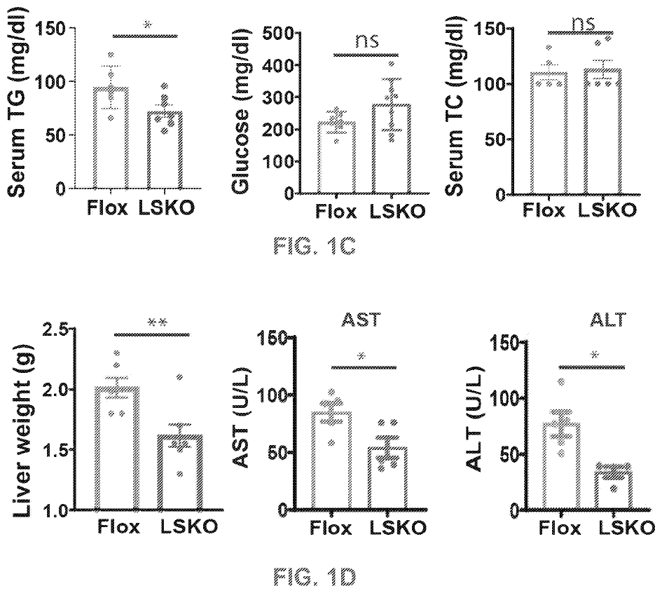

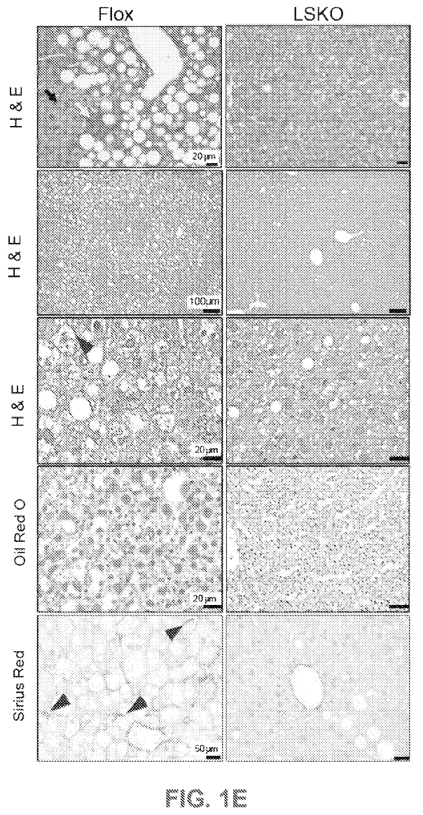

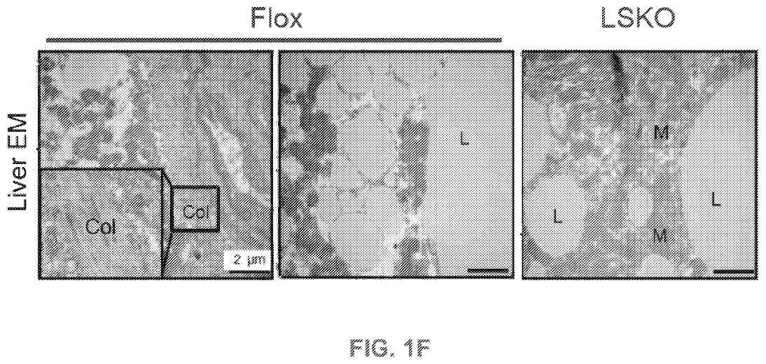

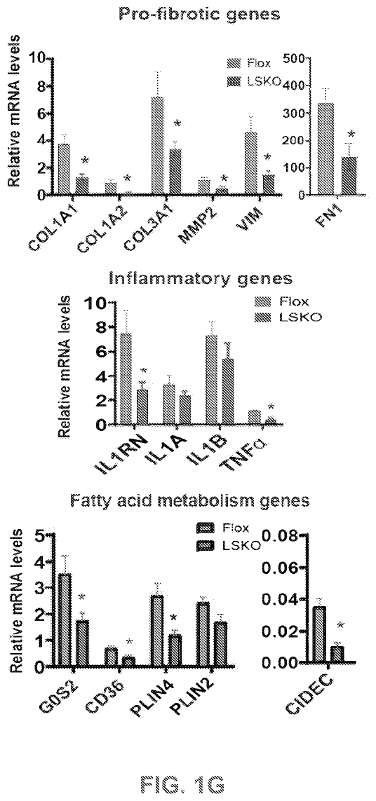

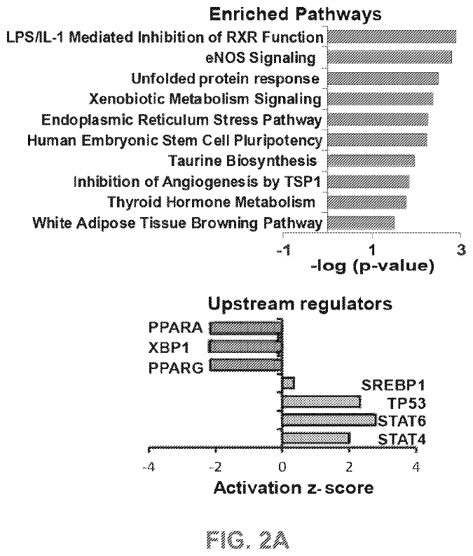

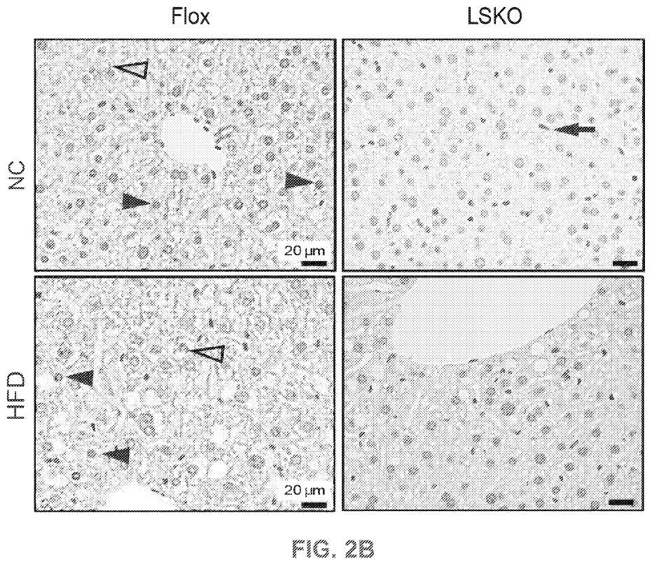

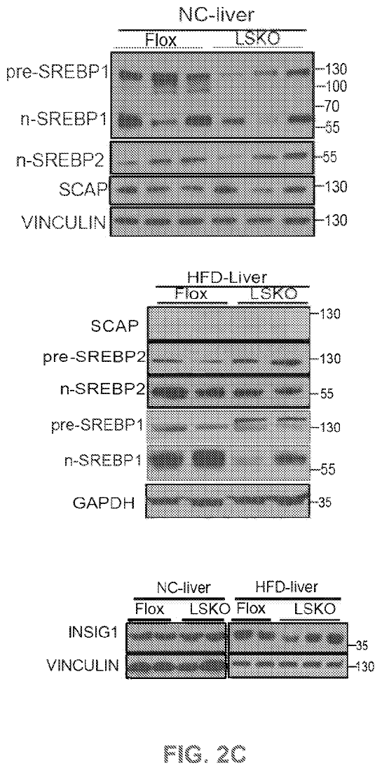

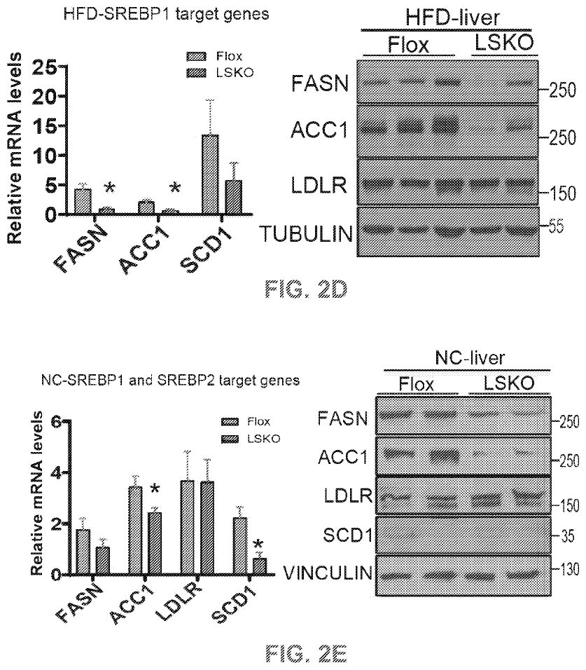

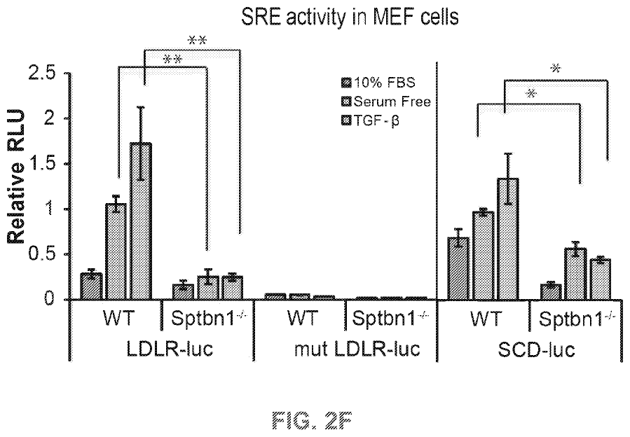

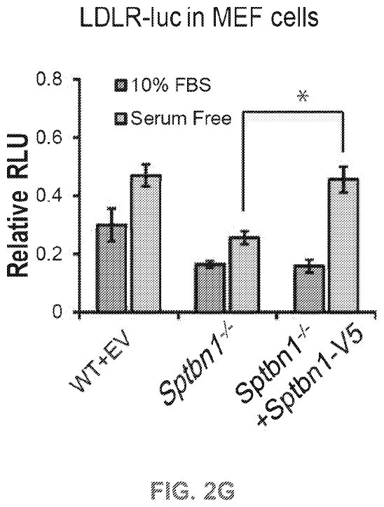

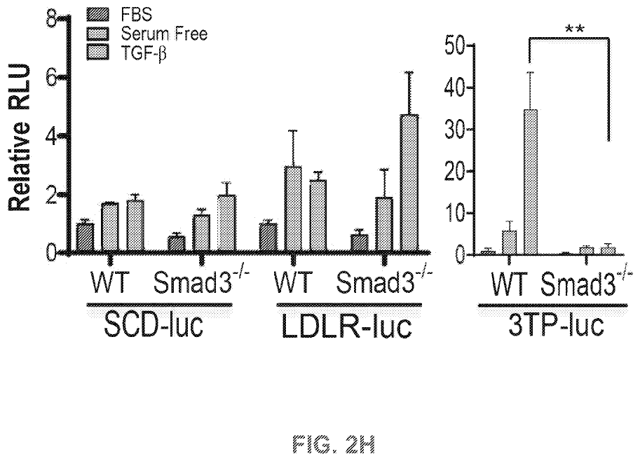

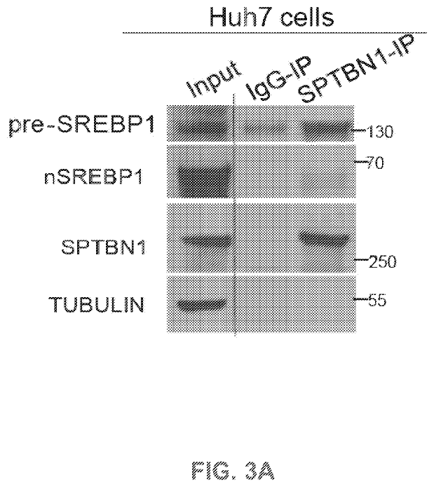

Liver-specific conditional SPTBN1 (Sptbn1-flox) knockout mice (LSKO) were generated without hepatocyte SPTBN1 (Supplementary FIG. 1A, B). LSKO mice are viable and fertile. Hepatocyte-specific deletion of SPTBN1 was confirmed by lack of detectable protein by immunoblot or immunohistochemical staining (Supplementary FIG. 1B, C. The liver histology and metabolic status were characterized for the LSKO mice fed a normal chow diet (Supplementary FIG. 1D-H). On normal chow, the LSKO mice were similar in body weight to the Flox control mice (Supplementary FIG. 1E), but the LSKO displayed significantly decreased concentrations of serum triacylglycerol (TG) (Supplementary FIG. 1F) and blood glucose (Supplementary FIG. 1G) with slightly increased insulin sensitivity in an insulin tolerance test (Supplementary FIG. 1H). Histologically, the livers of the LSKO mice had reduced lipid accumulation (Supplementary FIG. 1D) when compared to Flox mice. Additionally, the LSKO mice and Flox control mice had similar food and water intake and urine output (Supplementary FIG. 2A).

The LSKO mice were used to evaluate the effects of HFD. 10-12-week-old male and female Flox control and LSKO mice were placed on a HFD for 12 to 20 weeks (FIG. 1A). The HFD had 60% of calories from fat (29-31). Food and water intake and urine output were similar between the Flox control and LSKO mice after 12 weeks on a HFD (Supplementary FIG. 2A). However, after 12 weeks of HFD, LSKO mice gained less weight, had less visceral fat, and were protected from HFD-induced obesity (FIG. 1B). Metabolic profiling indicated that the HFD-fed LSKO mice had a lower concentration of serum triglyceride (TG) but were similar to the HFD-fed Flox control mice for serum glucose and total cholesterol concentrations (FIG. 1C). HFD-fed LSKO mouse livers were smaller (FIG. 1D) than those of HFD-fed Flox control mice, despite both mouse genotypes having comparable Ki67 labeling of proliferating cells in their livers (Supplementary FIG. 2B). Analysis of liver function by measuring serum aspartate transaminase (AST) and alanine transaminase (ALT) concentrations showed reduced concentrations in the LSKO mice, suggesting better liver function and less liver damage in HFD-fed LSKO mice compared to HFD-fed Flox control mice (FIG. 1D).

Consistent with less liver damage in the HFD-fed LSKO mice, liver histology of these mice revealed a normal liver architecture, minimal lipid accumulation, and the absence of possible ballooning or signs of inflammation in the liver (FIG. 1E). In the livers of the HFD-fed Flox mice, electron microscopy revealed fibrogenesis, indicated by collagen deposition at the extracellular matrix, and steatosis, indicated by the accumulation of lipid droplets; these changes were not present in the livers of the LSKO mice fed a HFD (FIG. 1F). Compared with livers from the Flox mice fed a HFD for 16 weeks, livers from the LSKO mice had decreased expression of TGF-β/SMAD3-regulated genes associated with liver fibrosis, altered expression of genes associated with inflammation, and reduced expression of genes encoding proteins involved in fatty acid metabolism (FIG. 1G).

The changes in the Flox control mice indicated that these mice developed NAFLD with progression to NASH by 16 weeks of HFD. Furthermore, the LSKO mice were protected from this diet-induced liver condition. The LSKO mice fed a HFD were found to be protected from development of obesity and NAFLD and progression to NAFLD, as well as the development of HCC.

### EXAMPLE 2

### SPTBN1 Regulates SREBP Pathways and Fatty Acid Metabolism

To understand how liver-specific loss of SPTBN1 protected mice from liver disease, targeted investigation was performed for specific key regulators in the Flox and LSKO mice fed a HFD for 16 weeks, and global liver transcript analysis of RNA-sequencing (RNA-seq) data was performed to explore differences in gene expression in the livers of Flox and LSKO mice after 16 weeks of HFD. Transcript and protein abundance of the transcriptional regulator C/EBPα were examined, which regulates adipogenesis (32), and uncoupling protein 2 (UCP2), which reduces mitochondrial ATP production and is associated with NASH (33). Neither of these were different in the livers of the HFD-fed Flox and LSKO mice at the level of transcript or protein abundance (Supplementary FIG. 2C). Bioinformatic analysis by Ingenuity Pathway Analysis (IPA) showed that several of the pathways associated with the differentially expressed genes were associated with stress or infection (FIG. 2A). Ingenuity “Upstream Regulator” analysis of the differentially regulated genes in the LSKO mice indicated an association with reduced or low activity of transcription factors that control lipid and cholesterol metabolism- SREBP, PPARγ (peroxisome proliferator activated receptor γ) and PPARα- and of XBP1, a transcription factor activated by ER stress (FIG. 2A). Given the association between loss of SPTBN1 and a reduction in the SREBP and PPARG lipid pathways in the liver, a limited mass spectrometry data was inspected to set for proteins that coimmunoprecipitated with V5-tagged SPTBN1 from stressed HEK293T cells and were implicated in de novo lipogenesis or its regulation (Table 3). Unexpectedly, SREBP1 was a potential binding partner for SPTBN1 based on those data. Thus, SREBP1 was focused on.

TABLE 3
Mass spectrometry Sptbn1 pull down in HEK293T Cells.

OST48_HUMAN
HNRH1_HUMAN
IF4A1_HUMAN
TBB5_HUMAN
AZI2_HUMAN
IMPG1_HUMAN
G3P_HUMAN
GLIS1_HUMAN
SHRM3_HUMAN
ALMS1_HUMAN
PHB_HUMAN
DNJC2_HUMAN
KPYM_HUMAN
SREBP1_HUMAN
CM035_HUMAN
ACTA_HUMAN
RSSA_HUMAN
ECHB_HUMAN
NONO_HUMAN

Because SPTBN1 has scaffolding functions in both the cytoplasm and the nucleus, the evaluation was conducted to see if SREBP1 distribution or abundance in the liver was altered in the LSKO mice, either fed normal chow or a HFD. In the livers of Flox mice fed normal chow, SREBP1 had an intense punctate distribution with few cells showing nuclear staining (FIG. 2B, lower). In mice fed a HFD for 12-16 weeks, most hepatocytes in the livers from Flox control mice had nuclear localized SREBP1, consistent with SREBP1 activation. SREBP1 staining was less intense in the livers from the LSKO mice fed either normal chow or a HFD, and in the livers from HFD-fed LSKO mice fewer cells had nuclear SREBP1, suggesting less SREBP1 was active (FIG. 2B). In mice fed a normal chow, no difference was detected in SREBP1 transcript abundance in the livers from Flox control and LSKO mice (Supplementary FIG. 2D), indicating that the reduction in SREBP1 abundance was not mediated at the level of gene expression.

There are 2 genes encoding SREBP1 and SREBP2, thus the abundance of pre-SREBP (ER-localized, full-length SREBP) and n-SREBP (cleaved, nuclear SREBP) were evaluated for both proteins, as well as for their ER-localized regulators SCAP and INSIG in the livers of Flox and LSKO mice fed normal chow or a HFD for 12-16 weeks. SCAP and INSIG abundance was similar between Flox and LSKO mice in the normal chow or HFD groups with SCAP showing a consistent decrease in abundance in the mice fed a HFD (FIG. 2C). In addition, knockdown of SPTBN1 by siRNA in THLE2 human hepatocytes did not affect INSIG1 abundance (Supplementary FIG. 2E). Although variable in the individual mice, compared with the abundance in the livers of Flox control mice fed normal chow, the abundance of pre-SREBP1 and n-SREBP1 appeared reduced in the livers of LSKO mice fed normal chow or a HFD (FIG. 2C). In mice fed a HFD, the abundance of pre-SREBP2 was similar or slightly less in the livers from Flox control mice, but the amount of n-SREBP2 appeared similar or slightly less in the LSKO mouse livers (FIG. 2C). Thus, the decrease in SREBP1 abundance detected by immunohistochemistry and immunoblot in LSKO mouse livers was not through changing the abundance of the SREBP ER-localized regulators or impairing SREBP1 gene expression.

To determine if the reduction in SREBP1 in LSKO mouse livers corresponded to a decrease in SREBP1 target gene expression, either transcript and protein abundance of products of de novo lipogenesis genes or performed luciferase reporter assays using SRE-containing promoters was analyzed. In livers of mice fed normal chow or a HFD for 12-16 weeks, ACC1 (encoding acetyl-CoA carboxylase 1), SCD1 (encoding stearoyl-CoA desaturase-1), and FASN (encoding fatty acid synthase) were less in the LSKO tissue, and these changes were reflected in less protein as well (FIG. 2D, E). Thus, reduced expression of SREBP1 target genes was detected in the absence of SPTBN1. Consistent with the minimal differences observed for SREBP2 abundance in the livers of Flox and LSKO mice, transcripts for LDLR, the main target of SREBP2 in cholesterol metabolism (34), were found to be similar the livers of the mice and the amount of LDLR protein was also found to be similar (FIG. 2D, E). The data suggested that SPTBN1 predominantly regulates SREBP1 and not SREBP2.

The SREBP-responsive luciferase assays used luciferase reporters containing wild-type SRE from the LDLR promoter region (LDLR-luc), SREBP-unresponsive mutant SRE (mut-LDLR-luc), or SRE from the SCD1 promoter (SCD-luc) and were performed in mouse embryonic fibroblasts (MEFs) cells isolated from wild-type mice and systemic Sptbn^{-/-}. Serum starvation, which activates SREBP in cultured cells (35), was found to increase SRE-dependent LDL-luc and SCD-luc activity in wild-type MEFs, and no or minimal SRE-dependent luciferase activity was found to be detected in Sptbn1^{-/-} cells (FIG. 2F), indicating impaired SREBP1-dependent target gene expression in the absence of SPTBN1. Re-expressing SPTBN1 in Sptbn1^{-/-} MEF cells restored SRE-dependent luciferase activity (FIG. 2G). The evaluation was conducted to see if TGF-β stimulated SRE-dependent gene expression and was found that TGF-β stimulated SRE-dependent luciferase activity in a manner dependent on SPTBN1 (FIG. 2F), suggesting that TGF-β signaling involving SPTBN1 could also contribute to de novo lipogenesis.

The examination was conducted to see if TGF-β signaling through SMAD3 affected SRE-dependent gene expression using the luciferase reporter genes expressed in WT or SMAD3^{-/-} MEFs. A trend was observed toward increased SRE-driven gene expression SMAD3^{-/-} MEFs (FIG. 2H, left). As a positive control, TGF-β stimulated luciferase expression was from a SMAD-dependent promoter in WT MEFs but not in the SMAD3^{-/-} MEFs (FIG. 2H, right). These data indicated that TGF-β signaling through SMAD3 is not responsible for the TGF-β-mediated enhancement of SRE-dependent gene expression.

### EXAMPLE 3

### SPTBN1 Binds to SREBP1

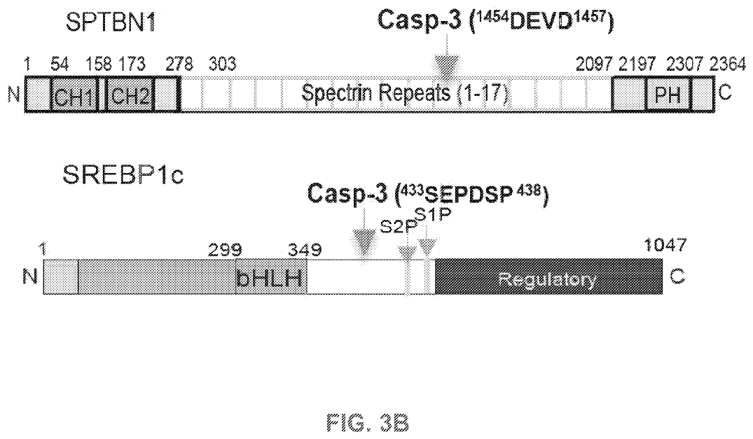

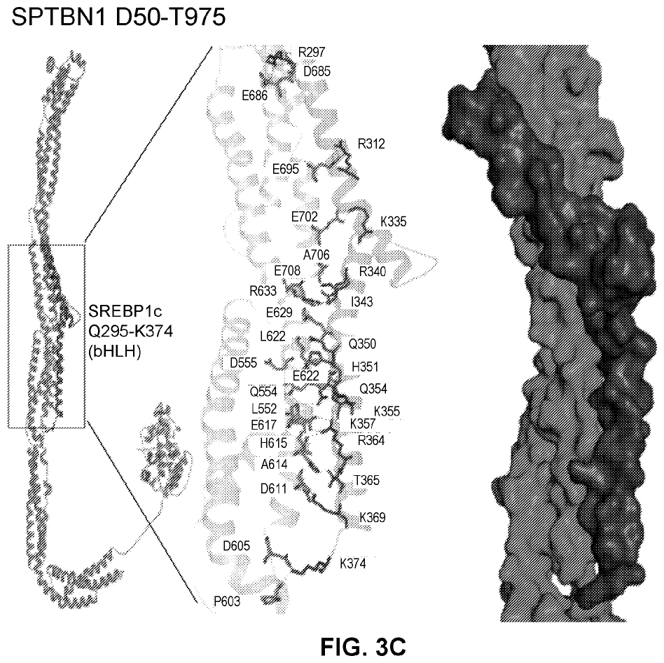

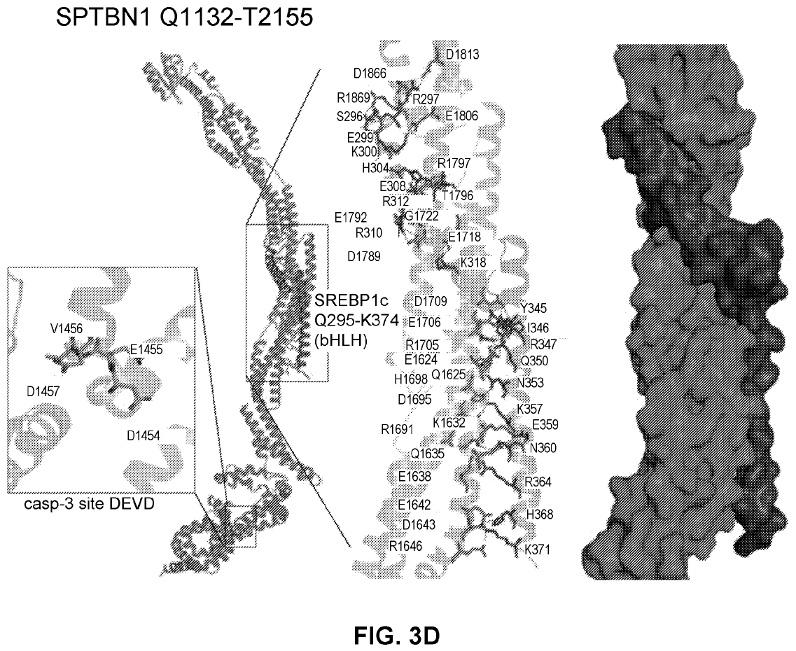

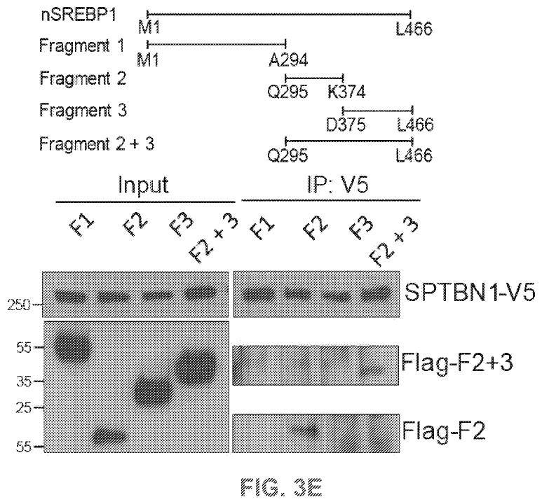

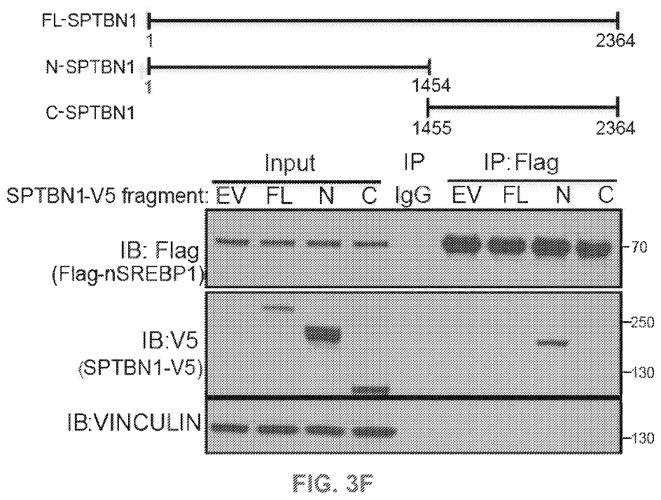

To verify the mass spectrometry data suggesting that SPTBN1 and SREBP1 were part of the same complex (table 3), SPTBN1 was immunoprecipitated from 3 HCC cell lines. Pre-SREBP1 and n-SREBP1 were detected in the immunoprecipitates from all 3 cell lines (FIG. 3A, Supplementary FIG. 3A) with the interaction between SPTBN1 and pre-SREBP1 occurring in the cytoplasm (Supplementary FIG. 3B).

To explore the structural basis of SPTBN1 and SREBP1 binding and map their potential interaction sites, the three-dimensional structures of SPTBN1 and SREBP1c were modelled and then structure-based molecular docking simulations were performed. There are 2 splice variants of SREBP1: SREBP1a and SREBP1c (36). SREBP1c was selected for the molecular docking simulations because this is the form that specifically stimulates fatty acid synthesis and is associated with liver steatosis (37).

SPTBN1 is comprised of 2,364 amino acid residues and characterized by multiple homologous tandem spectrin repeats, each composed of three antiparallel helices, flanked by a pair of calponin-homology (CH) domains at the N-terminal side and a pleckstrin homology (PH) domain at the C-terminal side (FIG. 3B). SPTBN1 has a caspase-3 cleavage site (1454DEVD1457) in the 11th spectrin repeat. SREBP1c is comprised of 1,047 amino acid residues that form an N-terminal DNA binding domain (bHLH), two transmembrane domains each with a cleavage site in the middle, and a regulatory domain at the C-terminus. SREBP1c has a caspase-3 cleavage site before the transmembrane domains (433SEPDSP438) (12) (FIG. 3B).

For the modeling, SPTBN1 was examined in three fragments covering D50-T975 and Q1132-T2155 (FIG. 3C) and A2198-K2364 (Supplementary FIG. 3C); SREBP1c was modelled in two fragments covering Q295-K374 (FIG. 3C) and P546-S705. SREBP1c P546-S705 can not be docked onto SPTBN1. However, the interactions were successfully simulated between the SREBP1c Q295-K374 fragment and SPTBN1 (FIG. 3C, D; Supplementary FIG. 3C; table 4).

The models predicted that SREBP1 c Q295-K374 has a high affinity conformational fit within a binding cavity of SPTBN1 and that the interactions are stabilized by multiple hydrogen bonds and Van der Waals interactions (table 4). On the basis of the lowest interaction energy values (38, 39), the predicted models were ranked as having the following affinities (table 4): SREBP1c Q295-K374/SPTBN1 Q1132-T2155 (FIG. 3D) >SREBP1c Q295-K374/SPTBN1 D50-T975 (FIG. 3C) >>SREBP1c Q295-K374/SPTBN1 A2198-K2364 (Supplementary FIG. 3C). Overall, the docking analysis predicted SPTBN1 fragments D50-T975 and Q1132-T2155 as preferred SREBP1c Q295-K374 binding sites.

TABLE 4
Predictive interacting residues
of SPTBN1 and SREBP1_C fragments

| | | | | Number of | Number of |
|---|---|---|---|---|---|
| | | | | SPTBN1 | SREBP1_C |
| | | | | residues | residues |
| Potential | Rep- | Weight- | Number | participating | participating |
| interacting | resen- | ed | of H- | in H-bond | in H-bond |
| residues | tative | Score | Bonds | interactions | interactions |
| SPTBN1 | Center | −843.3 | 30 | 19 | 14 |
| (D50-T975) - | Lowest | −962.2 | | | |
| SREBP1_C | Energy | | | | |
| (Q295-K374) | | | | | |
| SPTBN1 | Center | −1047.4 | 43 | 24 | 20 |
| (Q1132- | Lowest | −1047.4 | | | |
| T2155) - | Energy | | | | |
| SREBP1_C | | | | | |
| (Q295-K374) | | | | | |
| SPTBN1 | Center | −631.3 | 18 | 10 | 11 |
| (A2198- | Lowest | −691.5 | | | |
| K2364) - | Energy | | | | |
| SREBP1_C | | | | | |
| (Q295-K374) | | | | | |

To test the predicted models, SREBP1 fragments and SPTBN1 fragments were generated to identify the regions required for their interaction. N-SREBP1 (amino acids M1-L466) was started and four fragments (FIG. 3D) were generated. Fragment 2 of SREBP1 corresponds to the modeled portion (Q295-K374) and was most effectively coimmunoprecipitated with full-length V5-tagged SPTBN1 (FIG. 3D). Indeed, this region was required for the interaction. Caspase-3 cleaves SPTBN1 into two products, N-SPTBN1 (amino acids M1-D1454) and C-SPTBN1 (E1455-K2364), which are detected in acetaminophen-damaged livers (27). N-SPTBN1 and C-SPTBN1 were generated to be tagged with V5 and only N-SPTBN1 was found to coimmunoprecipitate with Flag-tagged n-SREBP1 (FIG. 3E). These data suggested SPTBN1 and SREBP1 can interact directly. From the modeling and fragment interaction analyses, conditions causing activation of caspase-3 in cells were hypothesized to result in cleavage of SPTBN1, which promotes an interaction between N-SPTBN1 and n-SREBP1.

### EXAMPLE 4

### Cell Stress Induces Caspase-3-Dependent Cleavage of Both SREBP1 and SPTBN1 in Human Hepatocytes and HCC Cells

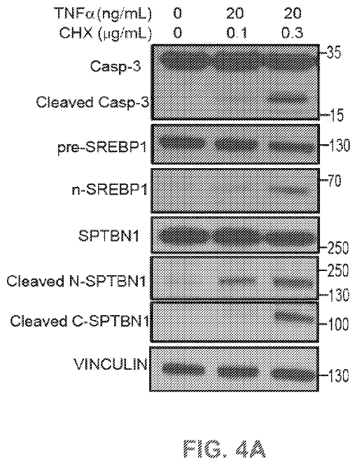

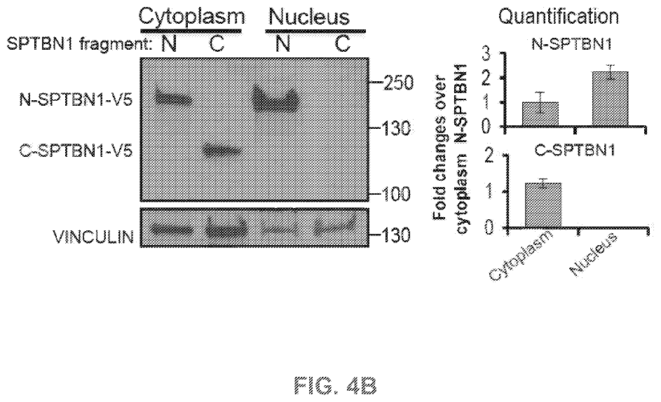

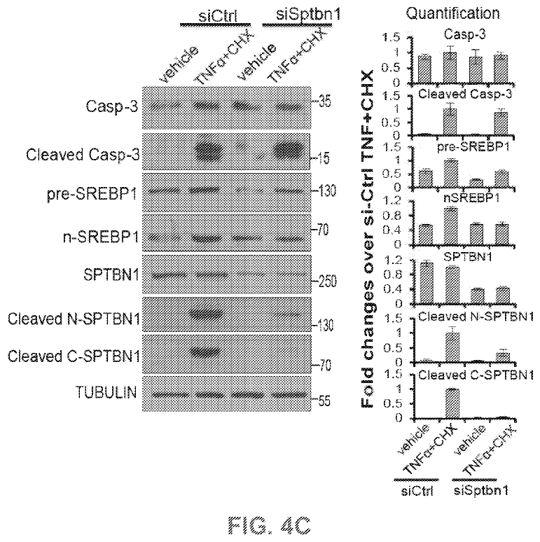

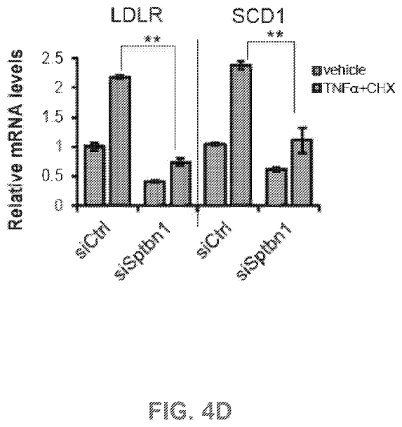

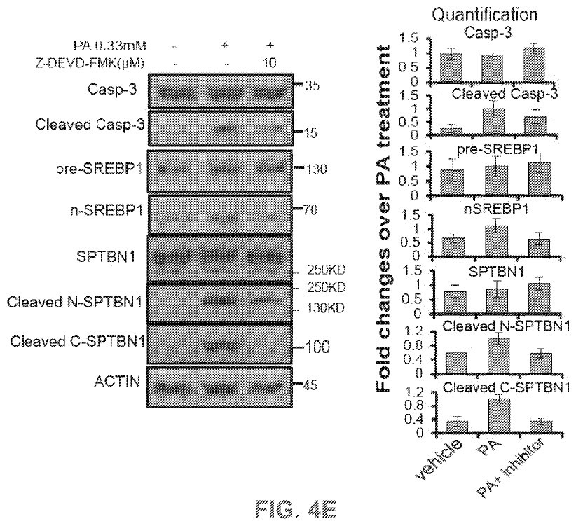

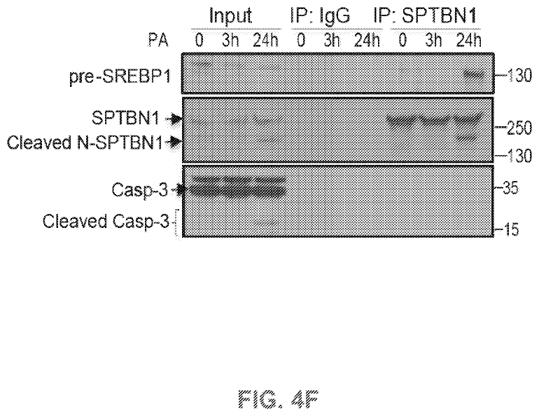

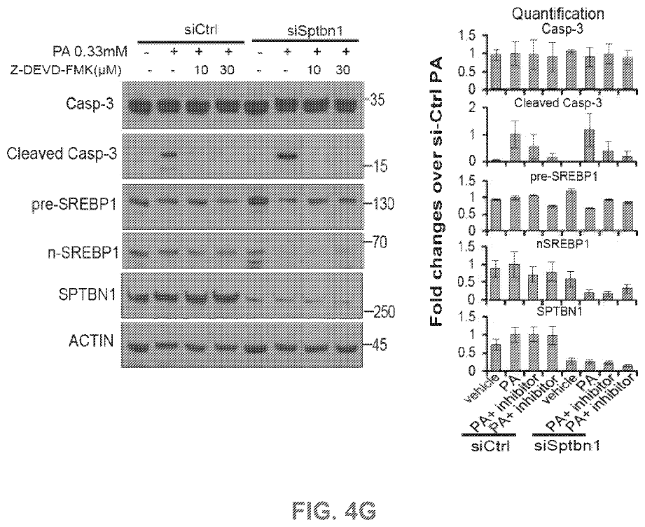

Stress-induced caspase activation is linked to the NASH phenotype (8, 9). The RNA-seq data revealed alteration in ER stress and UPR pathways in the livers of HFD-fed LSKO mice (FIG. 2A). Because both SREBP1 and SPTBN1 are caspase-3 substrates (12, 27) and the interaction data suggested that their cleavage products interacted (FIG. 3E, F), the relationship was explored among caspase-3, SPTBN1, and SREBP1 in the immortalized human hepatocyte cell line THLE2 and the HCC cell line Huh7. TNFα and cycloheximide or palmitic acid (PA) were used to induce caspase-3 activation. TNFα and PA induce caspase-3 activation in liver cells (40, 41) and are associated with NASH development (42, 43). THLE2 cells exposed to TNFα and cycloheximide exhibited caspase-3 activation (cleaved caspase-3), SREBP1 activation (n-SREBP1), and cleavage of SPTBN1 to produce both N-SPTBN1 and C-SPTBN1 (FIG. 4A). Analysis of the cytosolic and nuclear distribution of N-SPTBN1 and C-SPTBN1, expressed as V5-tagged proteins in THLE2 cells, showed that N-SPTBN1 was present in both the cytoplasm and nucleus, whereas C-SPTBN1 was only detected in the cytoplasm (FIG. 4B). Knockdown of SPTBN1 in Huh7 cells did not impair caspase-3 activation induced by TNFα and cycloheximide or cleavage of SREBP1 in these conditions (FIG. 4C). However, expression of SCD1, a SREBP1 target gene, was reduced (FIG. 4C). In this HCC cell line exposed to TNFα and cycloheximide, impaired expression of the SREBP2 target gene LDLR was observed (FIG. 4D), which contrasts with the findings in the livers of normal chow-fed LSKO mice (FIG. 2E) and likely relates to the lack of stress in the in vivo condition.

Exposing Huh7 cells to PA induced caspase-3 activation, which was reduced by caspase-3 inhibitor (Z-DEVD-FMK) (FIG. 4E). PA also triggered cleavage of SPTBN1, which was also reduced by the caspase-3 inhibitor, indicating that STPBN1 cleavage depends on caspase-3 activity (FIG. 4E). Pre-SREBP1 and SPTBN1 and N-SPTBN1 were found to coimmunoprecipitate from Huh7 cells exposed to PA, consistent with an interaction between SREBP1 and SPTBN1 occurring with the caspase-3 cleavage product N-SPTBN1 (FIG. 4F). N-SREBP was found to be reduced in PA-treated cells in which SPTBN1 was knocked down even though caspase-3 activation was unaffected, suggesting that the interaction with SPTBN1 may stabilize n-SREBP1 (FIG. 4G).

In stressed hepatocytes or hepatocytes cultured with high amounts of PA, caspase-3 cleaved both SREBP1 and SPTBN1 and the cleaved products interacted to stabilize the nuclear form of SREBP1, thereby promoting de novo lipogenesis.

The biochemical data provided a mechanistic link between nonapoptotic caspase-3 activity and de novo lipogenesis through formation of nSREBPs and stabilization of nSREBP1 by interaction with caspase-cleaved N-SPTNB1. This provides a second pathway, in addition to ER stress-induced activation of SREBPs, for aberrant activation of de novo lipogenesis that results in steatosis and development of NAFLD. The data suggested that caspase-mediated activation of SREBP1 was independent of changes in SCAP and INSIG, the ER-localized regulators of SREBP activation (6, 52); thereby bypassing the normal controls that limit de novo lipogenesis under conditions of sufficient or excess lipids.

### EXAMPLE 5

Human NASH is Associated with Increased SPTBN1 and CASPASE3 Expression and Increased TGF-β Pathway and SREBP1 Activity

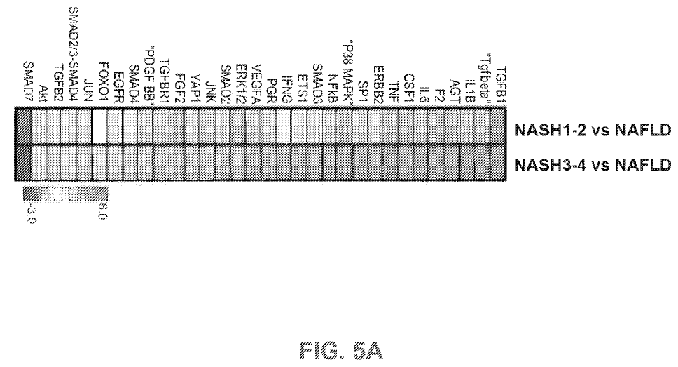

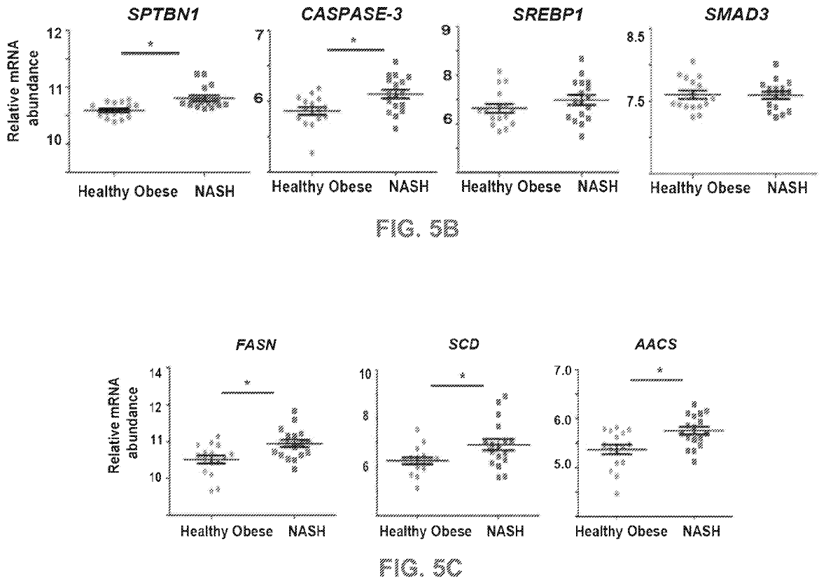

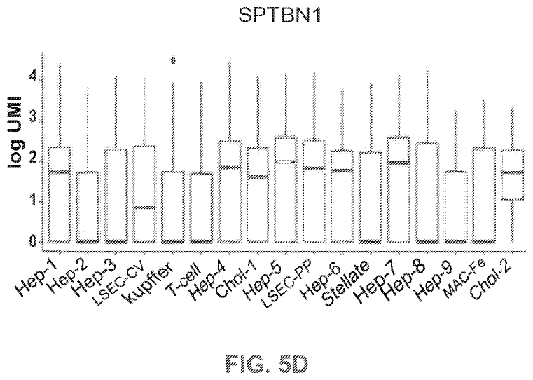

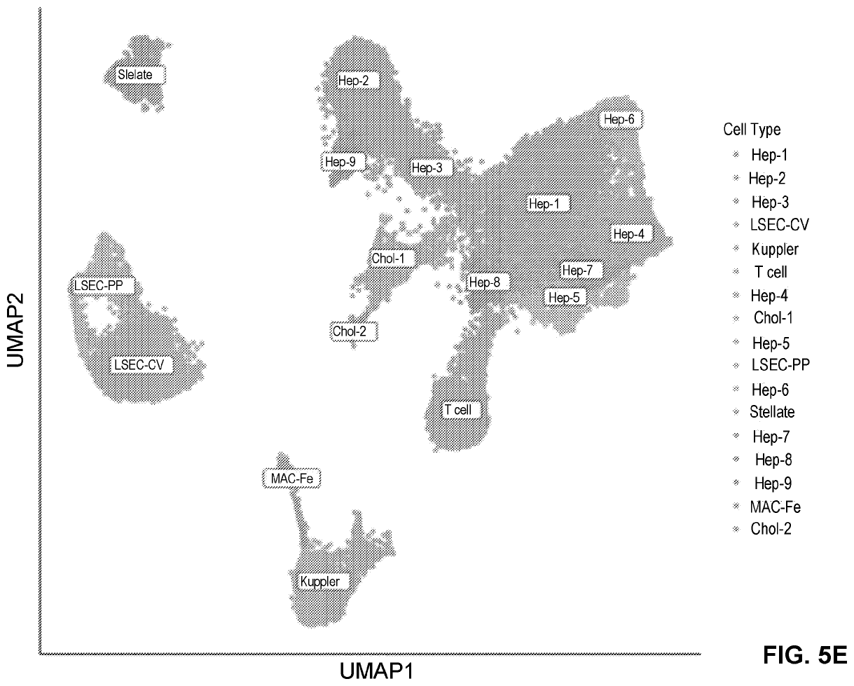

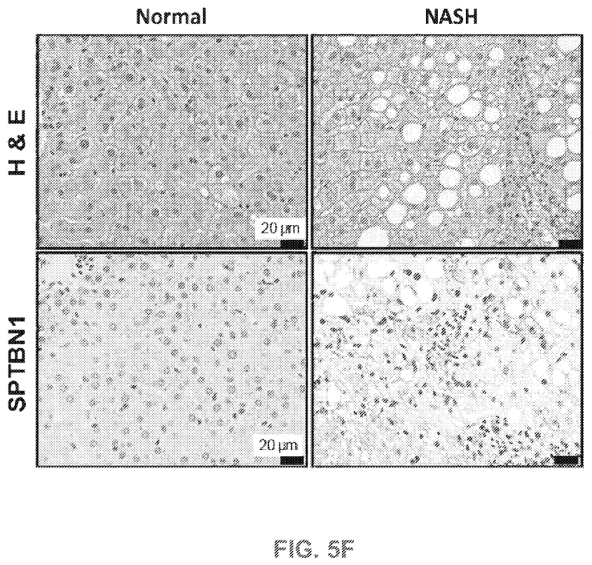

To gain insight into the relevance of the findings implicating SPTBN1 and a pathway involving caspase-3-mediated interaction between cleavage products of SPTBN1 and SREBP1 in steatosis, data were analyzed from four publicly available databases with information on healthy obese subjects and patients with NAFLD, NASH, or HCC. Ingenuity “Upstream Regulator” analysis was used to evaluate proteins associated with differences in gene expression between early stages of NASH (NASH1 and 2) and NAFLD and between late stages of NASH (NASH3 and 4) and NAFLD. Consistent with the involvement of TGF-β signaling in fibrosis, a key indicator of the progression of NASH from NAFLD (14, 44, 45), the upstream regulators with increased activity included several proteins in the TGF-β pathway, and the only downregulated protein was SMAD7, an inhibitor of TGF-β signaling (FIG. 5A).

Liver tissue data from healthy obese were compared with that from NASH patients for transcripts for SPTBN1, CASPASE-3, SREBP1, and SMAD3, as well as for SREBP1 target genes involved in lipogenesis. Both SPTBN1 and CASPASE-3 transcripts were increased in NASH patients, whereas no differences were observed for SREBP1 and SMAD3 (FIG. 5B). Indicative of increased SREBP1 activity, significantly increased expression of FASN, SCD, and AACS was found (FIG. 5C).

Because NASH represents a high risk for progression to HCC (1), single-cell RNA-seq was performed for SPTBN1 expression in liver tissue from patients with both NASH and HCC but without cirrhosis. A subset of cells was identified with high SPTBN1 expression (FIG. 5D, table 1): 5 subtypes of hepatocytes (Hep-1, Hep-4, Hep-5, Hep-6, and Hep-7), both types of cholangiocytes (Chol-1 and Chol-2), and liver sinusoidal endothelial cells of the portal vein (LSEC-PP). Analysis of the single-cell RNA-seq data showed that the hepatocytes with high SPTBN1 expression clustered together when plotted on a phenograph (FIG. 5E). SPTBN1 abundance was confirmed to be greater in liver tissue from NASH patients than in liver tissue from healthy controls (FIG. 5F).

Collectively, this evaluation of human NASH liver data is consistent with the findings from the HFD-fed mice that implicate SPTBN1 in progression of this condition. Furthermore, these data confirmed increased SPTBN1 and caspase-3, along with increased activity of TGF-β/SMAD signaling and SREBP1-dependent gene expression, in NASH.

### EXAMPLE 6

Targeting SPTBN1 by siRNA In Vivo Attenuates HFD-Induced NAFLD and NASH

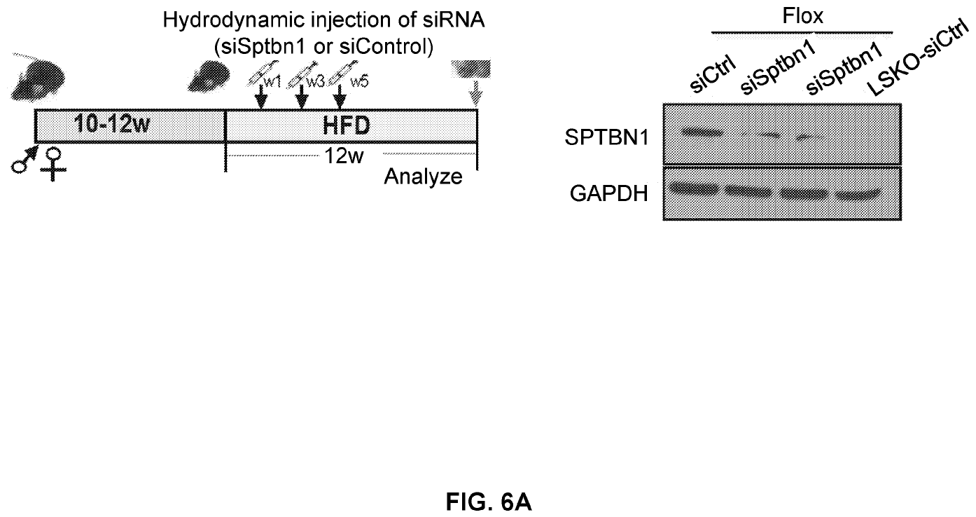

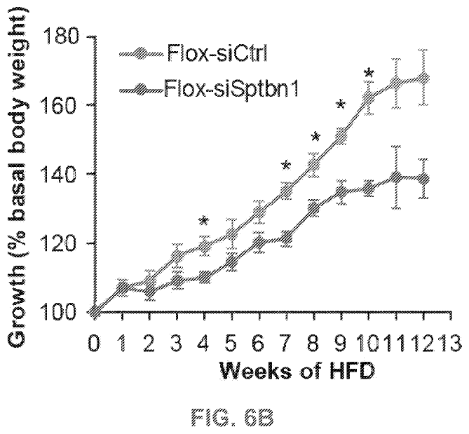

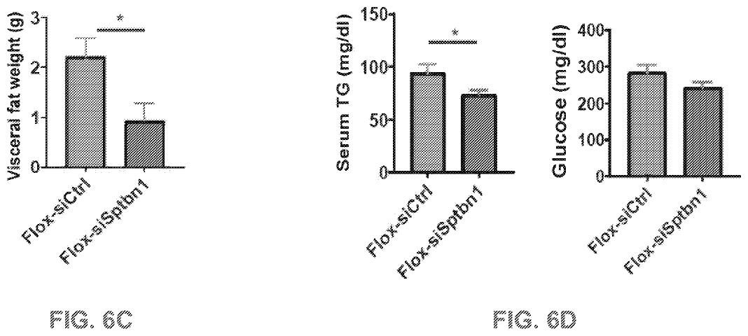

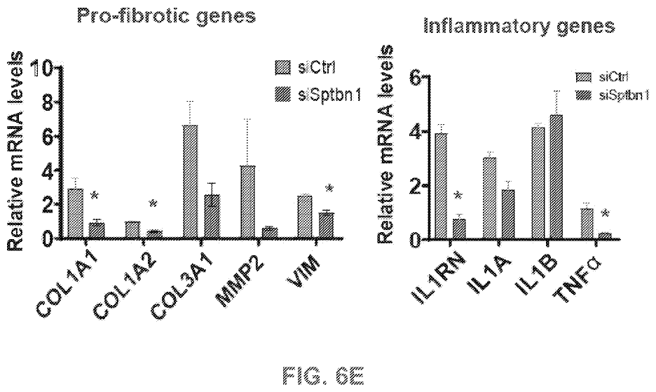

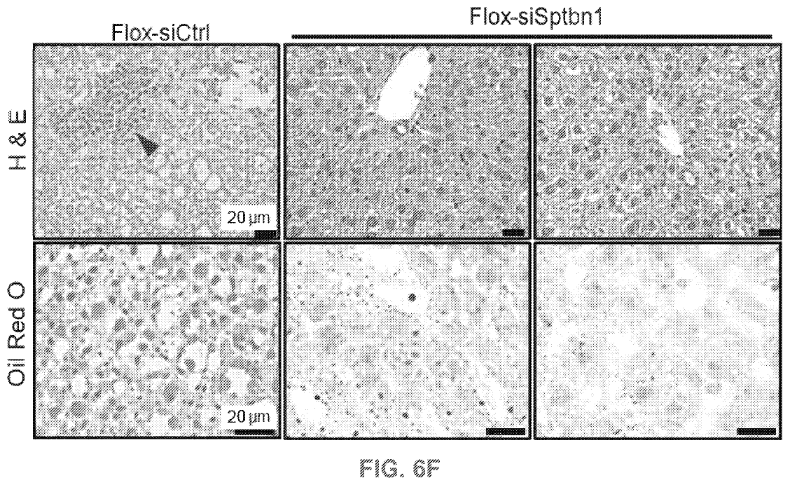

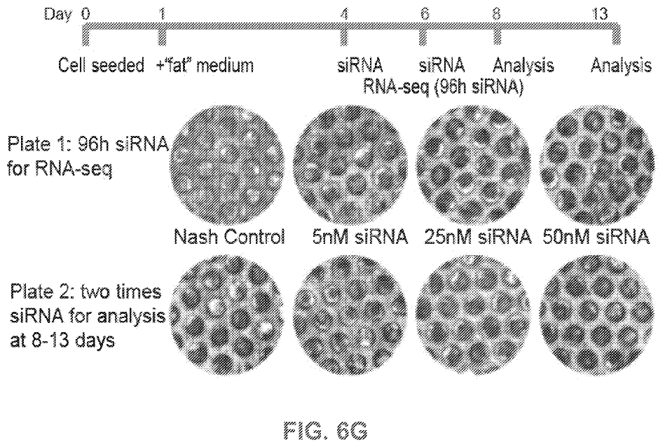

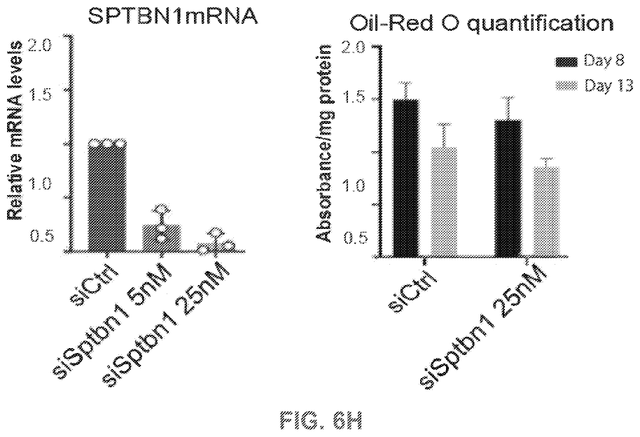

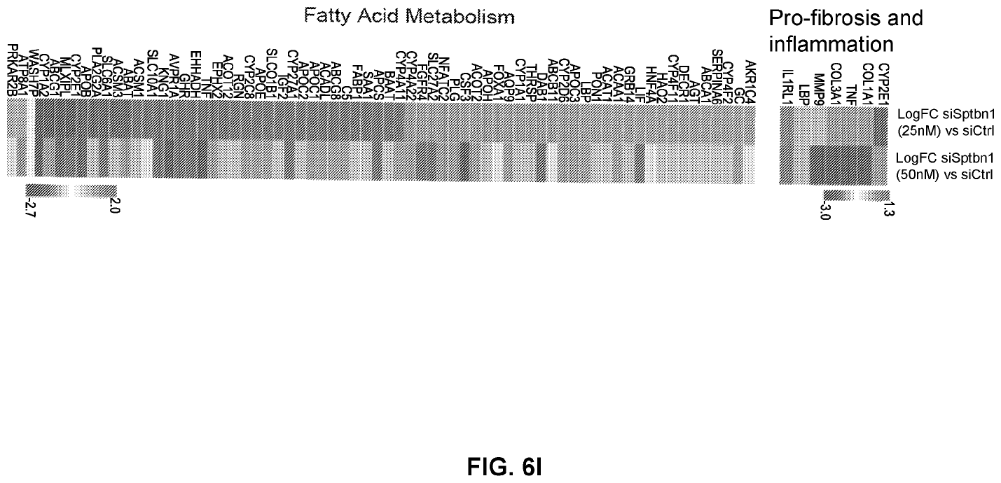

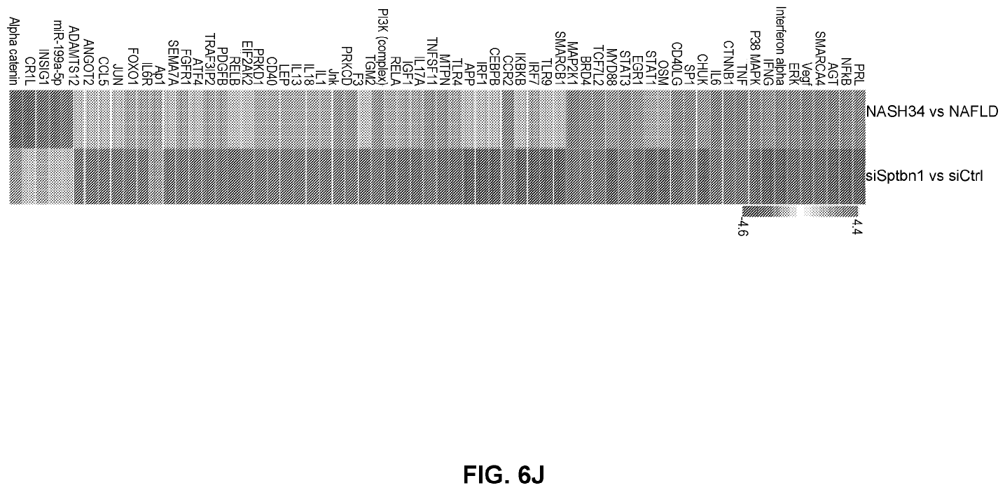

Liver diseases are one of the few that have FDA-approved siRNA-based treatments (46). Therefore, an examination was conducted to see if siRNA-mediated knockdown of SPTBN1 in Flox mice protected them from HFD-induced NAFLD and NASH. Flox mice (10-12 weeks old) were fed a HFD for 12 weeks. One week after start of the HFD, mice were injected hydrodynamically with either siRNA targeting SPTBN1 (siSptbn1) or an equivalent volume of siRNA negative control (siCtrl) every two weeks for a total of 3 injections (FIG. 6A). Mice were monitored weekly for body weight gain and survival. Knockdown of SPTBN1 was confirmed by siSptbn1 in the livers of the Flox mice at the end of the experiment (FIG. 6A, right). For each of the parameters, the siSptbn1-treated mice responded similarly to the LSKO mice to HFD. Compared to the siCtrl-treated mice, the siSptbn1-treated mice gained significantly less weight (FIG. 6B) and accumulated less visceral body fat (FIG. 6C), had lower blood TG concentrations, and similar blood glucose concentrations (FIG. 6D). Histology, lipid accumulation, and expression of pro-fibrotic genes and inflammatory genes in the livers of the siSptbn1-treated mice were also similar to those of the LSKO mice with significantly less expression of pro-fibrotic genes and inflammatory genes than in the livers from siCtrl-treated mice (FIG. 6E) and normal liver architecture and low lipid accumulation without any signs of NAFLD or NASH (FIG. 6F).

The same benefit of siSPTBN1 was observed in liver-specific SPTBN1 heterozygous mice (LHET) (Supplementary FIG. 4A, B) and in female Flox mice (Supplementary FIG. 4C). Thus, siSPTBN1 treatment prevented NAFLD development and progression to NASH in mice when started early in a period of HFD consumption.

Hepatocyte-specific absence of SPTBN1 had obviously no detrimental effects on liver morphology or function, and siRNA targeting SPTBN1 was an effective therapeutic in a preclinical mouse NASH model and in reducing HCC development in a chemically induced mouse model. Additionally, acetaminophen is a liver-toxic, commonly used over-the-counter medication (51). Low doses of acetaminophen induce phosphorylation of TGFBR2 and SMAD signaling with higher toxic doses promoting caspase activation and leading to production of caspase-cleaved SPTBN1 and severe hepatotoxicity (27). The data suggested that targeting SPTBN1 could be a useful therapy to prevent acetaminophen liver toxicity.

The results placed SPTBN1 as a central player in both steatosis and fibrosis in response to HFD: as a participant in TGF-3/SMAD3 signaling, SPTBN1 promotes fibrosis and, as a participant in stress-activated SREBP1 signaling, SPTBN1 promotes de novo lipogenesis and steatosis.

### EXAMPLE 7

SPTBN1 Knockdown in a Human 3D-Culture NASH Model Reduces Transcriptional Changes Associated with NASH

The mouse results showed that SPTBN1 knockdown prevented HFD-induced NAFLD and progression to NASH. This potential therapy was also tested in a newly developed 3D perfused microphysical system that enables the co-culture of primary human hepatocytes, hepatic Kupffer cells, and stellate cells in medium enriched in fatty acids, sugars, and insulin for 2 weeks as a culture model of human NASH (47, 48). The 3D cultures were exposed to different concentrations of siSptbn1 and equivalent concentrations of siCtrl, applying the siRNA on day 4 and on day 6 (FIG. 6G). More than 90% decrease was observed in SPTBN1 transcripts after 96 h with the 25 nM siRNA treatment (FIG. 6H, left). No significant difference was found in lipid accumulation at day 8 or day 14 (2 days or 7 days after the addition of the siRNA); however, the amount was trending downward in the siSptbn1-treated cultures (FIG. 6H, right).

RNA-seq analysis of cultures exposed to siSptbn1 (25 nM or 50 nM) or siCtrl days was performed on samples collected 96 h after the siRNA treatment. Pathway analysis revealed significant decreases in the siSptbn1-treated cultures in transcripts encoding proteins involved in fatty acid metabolism, including those involved in lipid transport, triglyceride and glycogen metabolism, and lipoprotein catabolism (FIG. 6I). A reduction was found in the transcripts encoding proteins involved in fibrosis and altered inflammatory gene expression in the siSptbn1-treated cultures (FIG. 6I). These changes indicated that si SPTBN1 treatment reduced de novo lipogenesis, inflammation, and fibrosis. As expected, a marked decreased was noted in a gene expression signature associated with TGF-β signaling in siSptbn1-treated cultures (Supplementary FIG. 4E), consistent with the mouse models showing that this profile was reduced in LSKO and was activated in mouse (HFD-induced NASH) and human NASH (Supplementary FIG. 4E).

“Upstream Regulators” analysis with IPA indicated that regulators with higher activity in human NASH compared to NAFLD were uniformly associated with lower activity in the siSptbn1-treated cultures compared to the siCtrl-treated cultures (FIG. 5J). Furthermore, upstream regulators with lower activity in NASH versus NAFLD had higher activity in the siSPTBN1-treated cultures versus the siCtrl-treated cultures (FIG. 5J). Thus, siSptbn1 appeared to reverse or counter NASH-associated regulatory changes, further supporting the concept of siSptbn1 as a potential therapy.

### EXAMPLE 8

LSKO Mice are Protected Mice from HFD-Induced HCC

The data indicated that the complete liver-specific loss of SPTBN1 protects mice from HFD-induced NASH, a well-established risk factor for HCC (1). Here, the implications of homozygous loss of hepatic SPTBN1 were evaluated on HCC. First, the HCC incidence was explored in LSKO mice and LHET mice fed a HFD for 20 weeks. Whereas two tumor nodules were found in one of four HFD-fed LHET mice, no liver tumors were found in any of the age-matched, HFD-fed LSKO mice (FIG. S7A). Because HCC development did not occur with sufficient frequency in this experimental condition, a chemically induced HCC model was switched to.

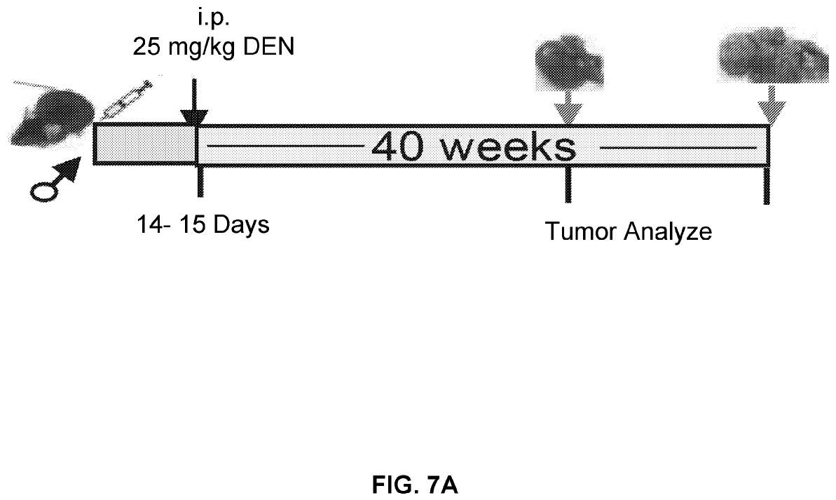

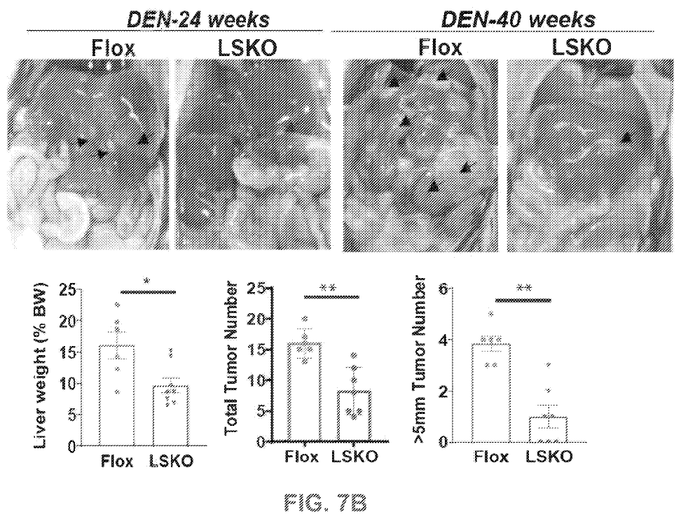

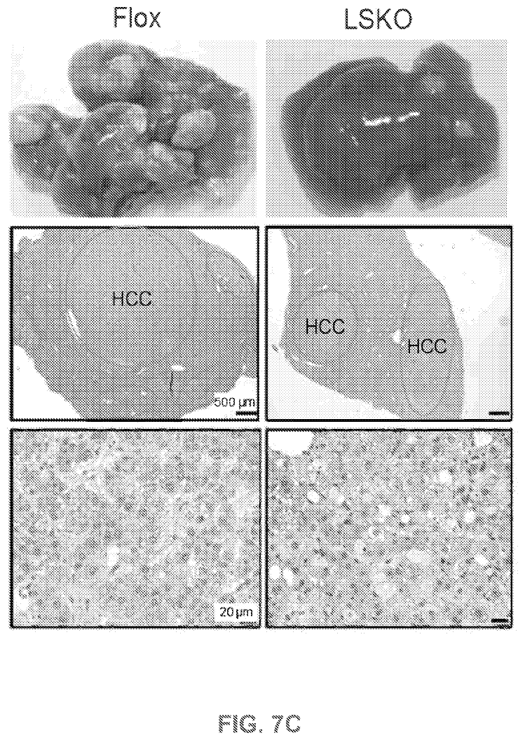

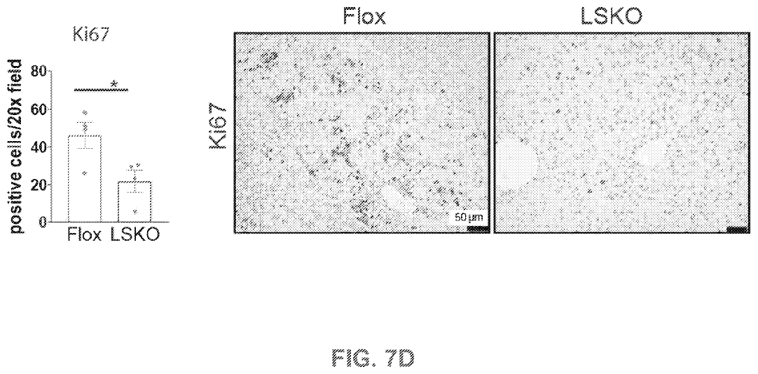

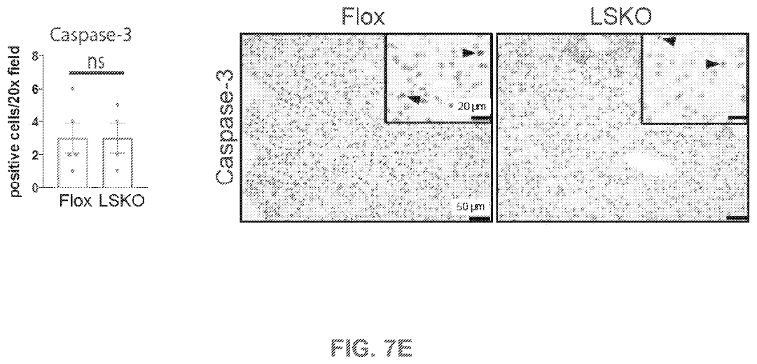

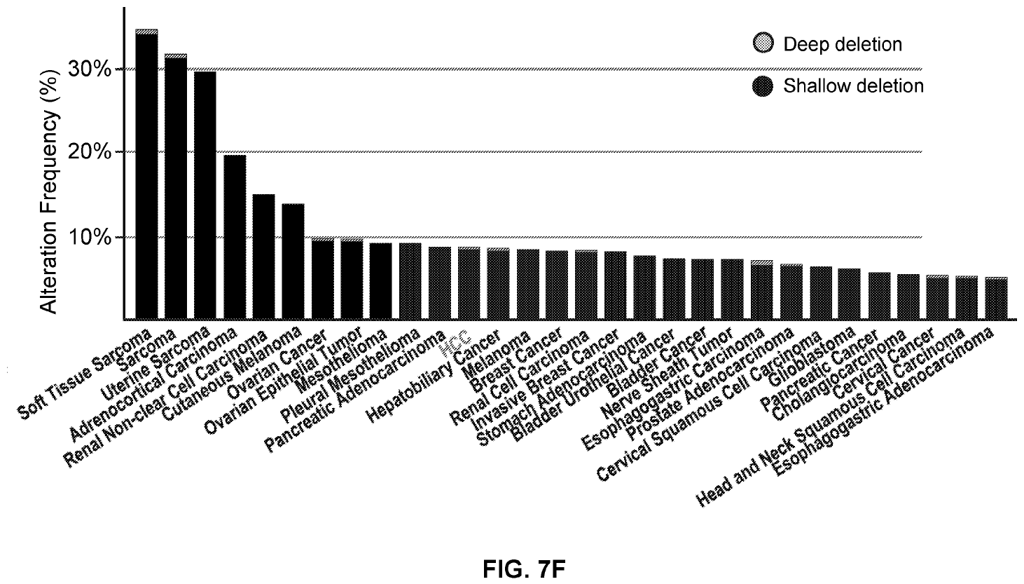

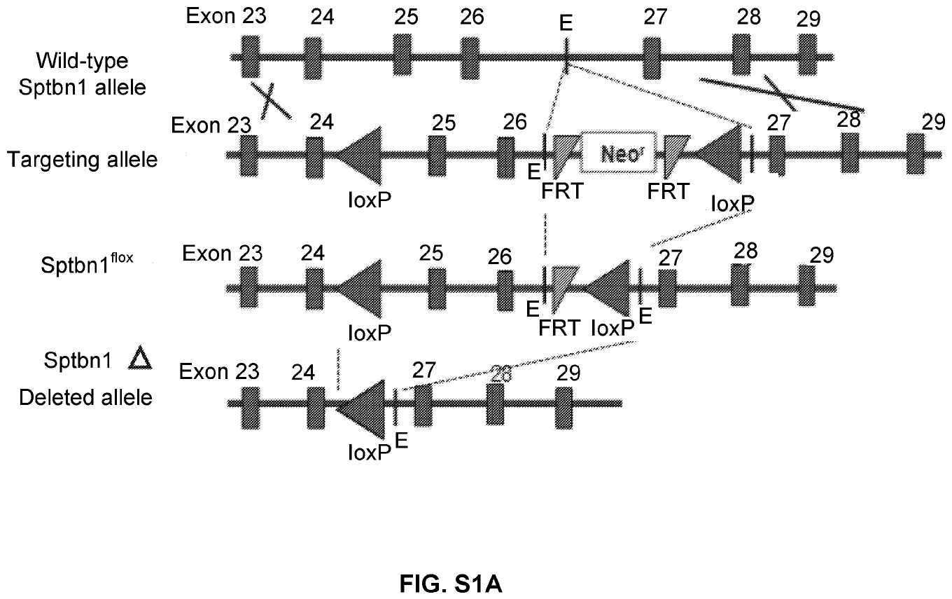

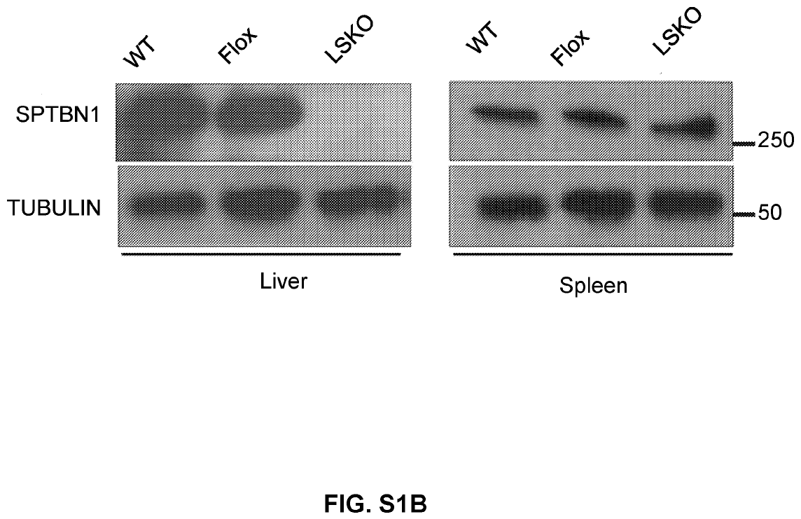

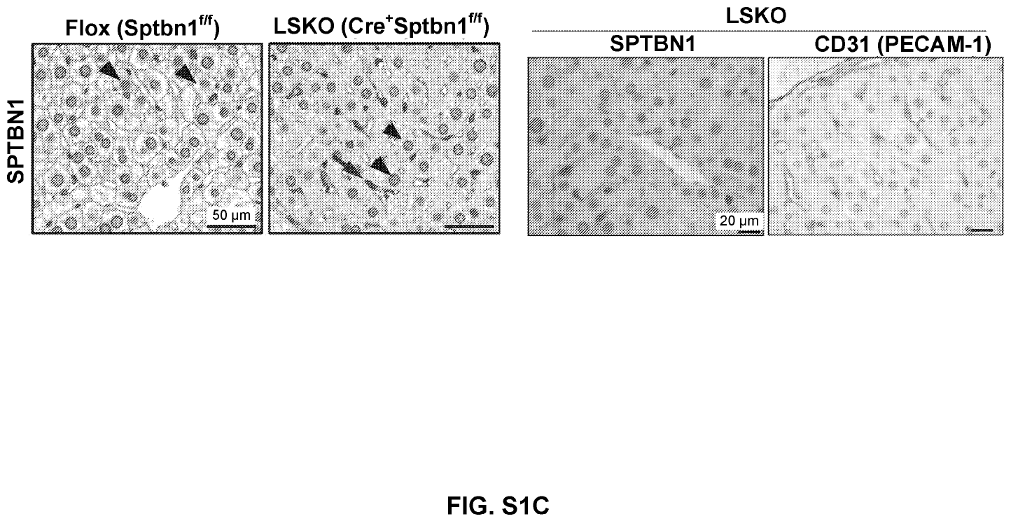

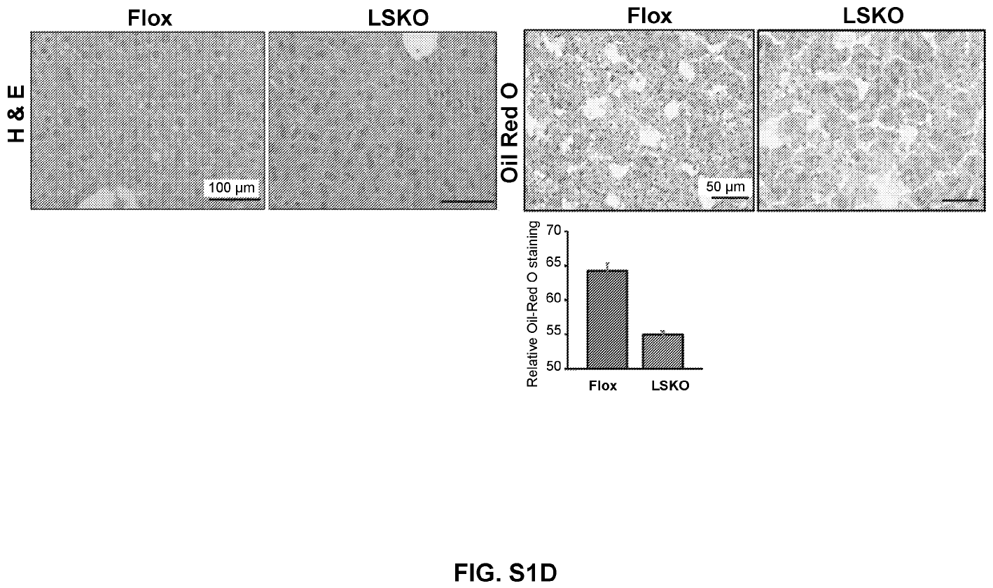

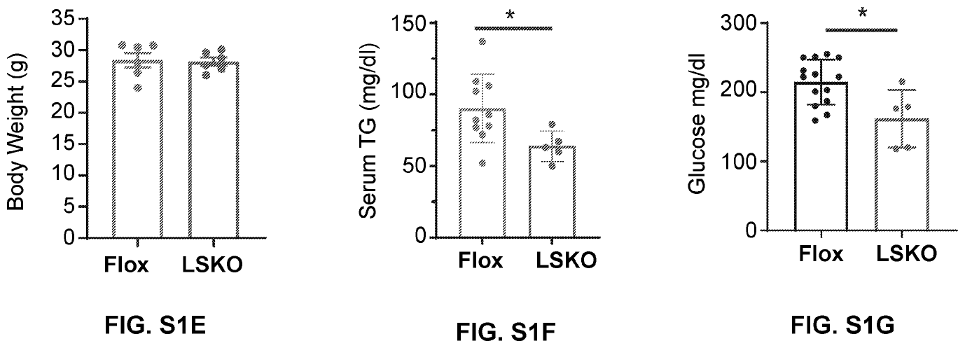

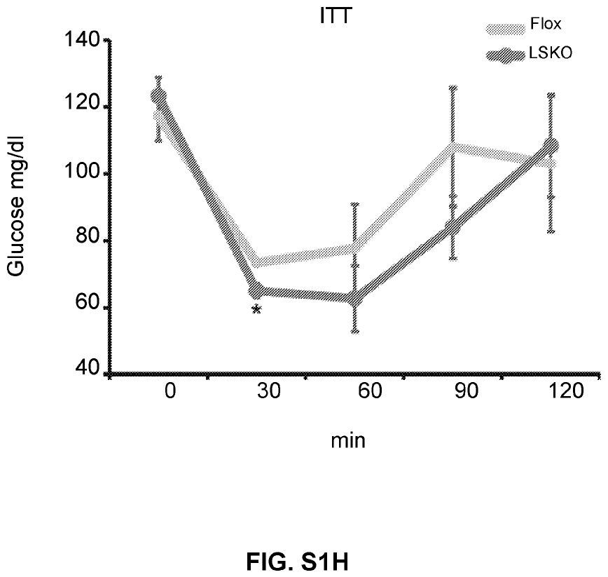

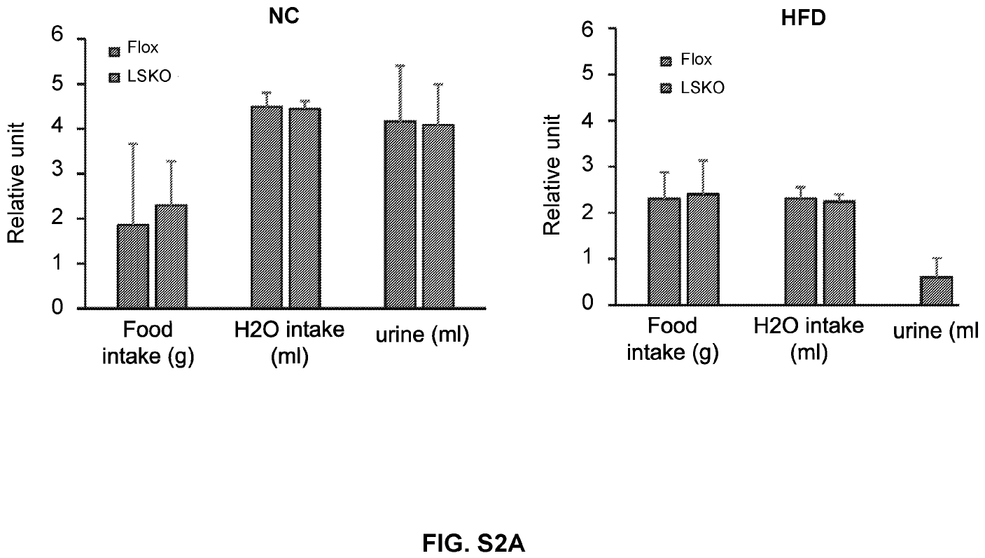

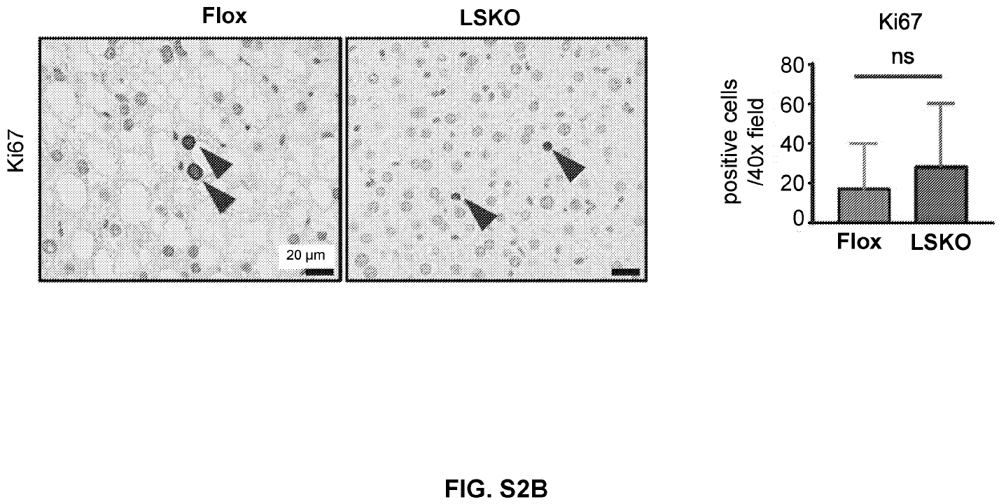

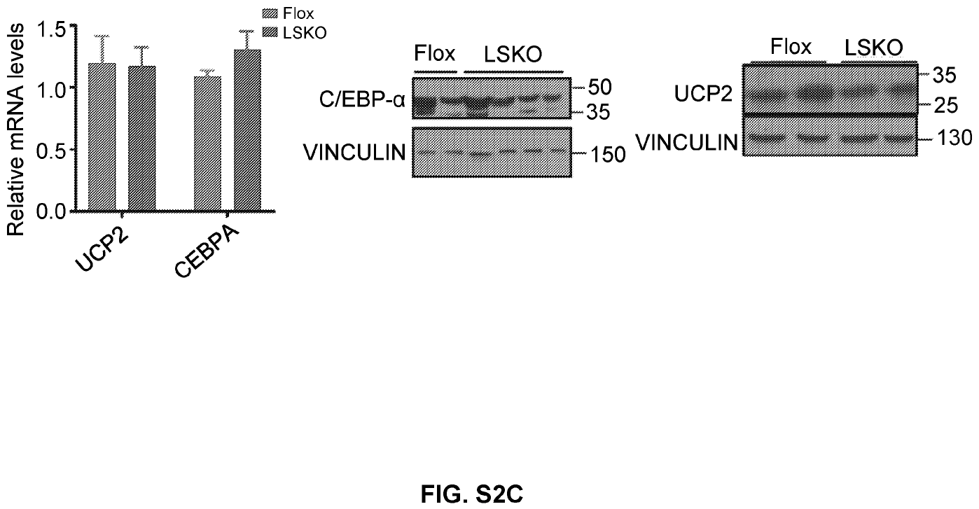

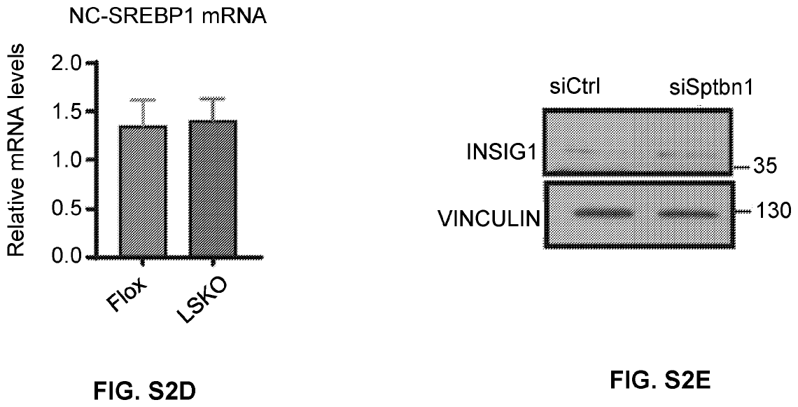

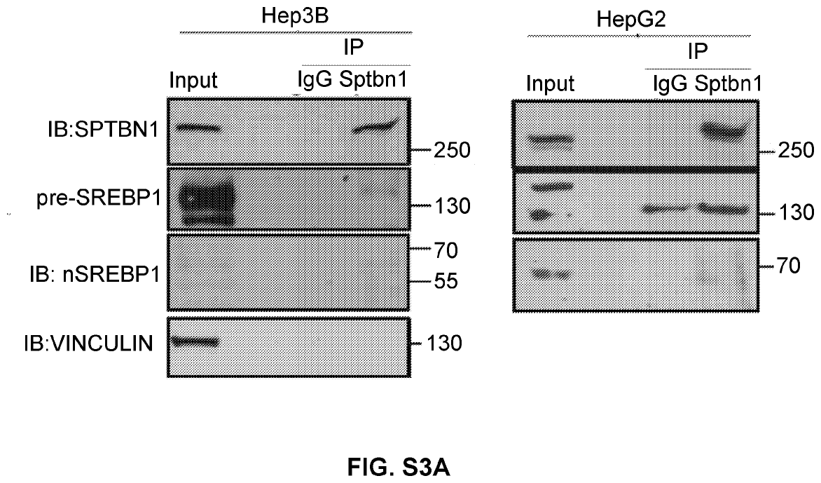

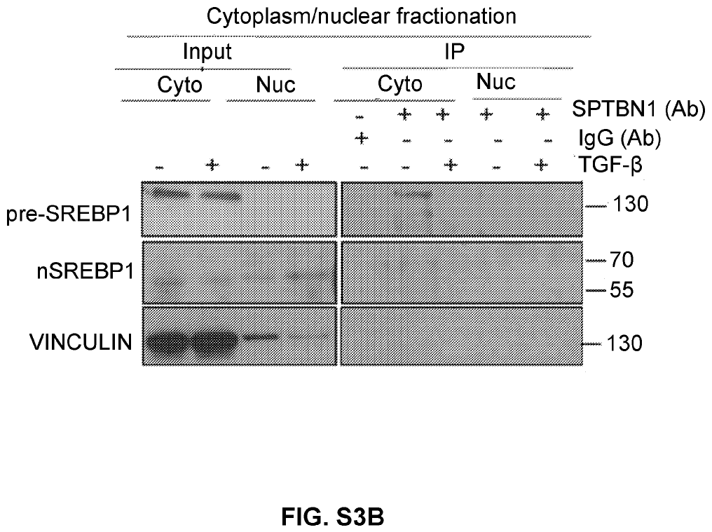

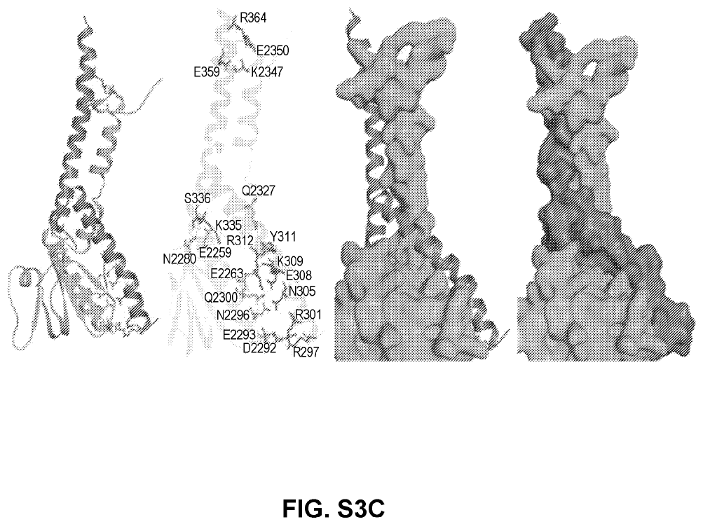

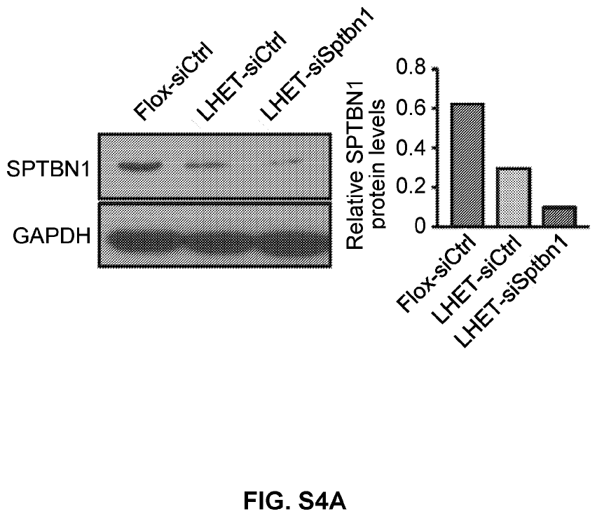

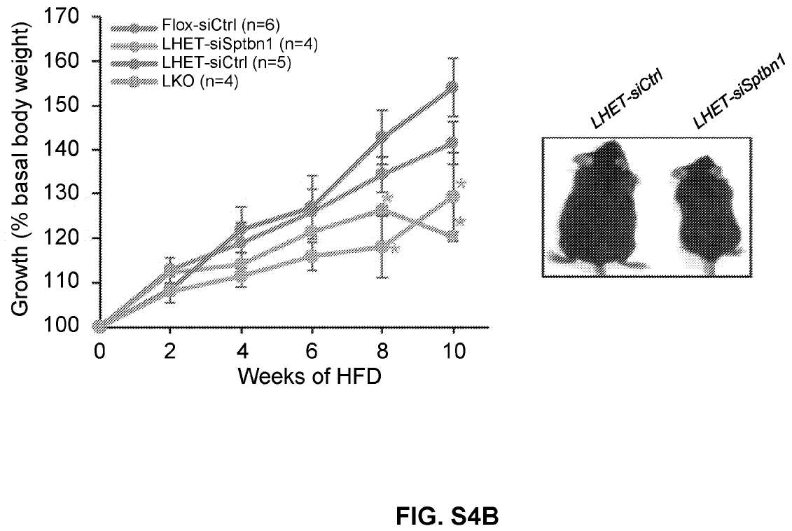

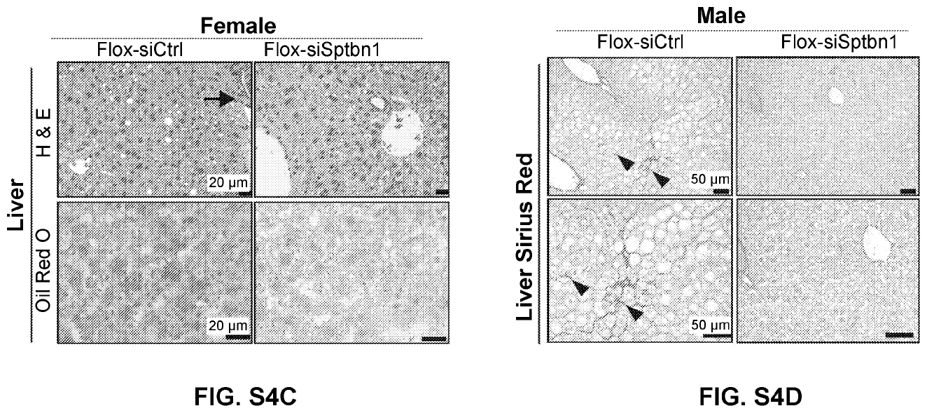

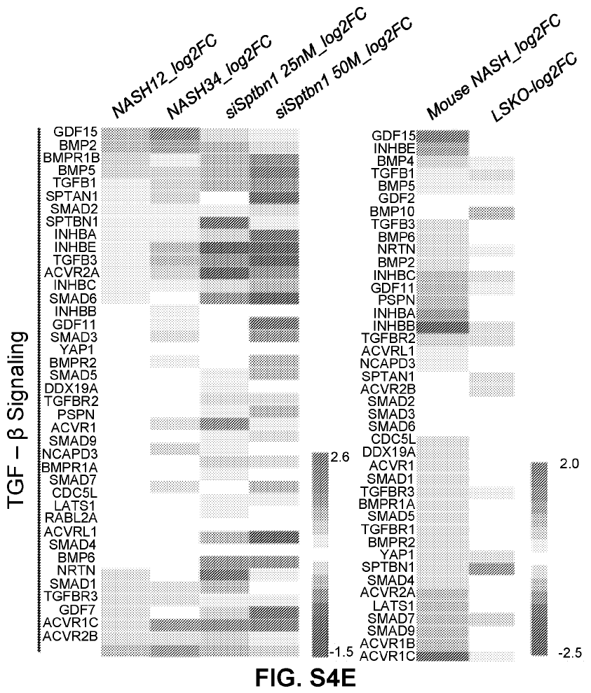

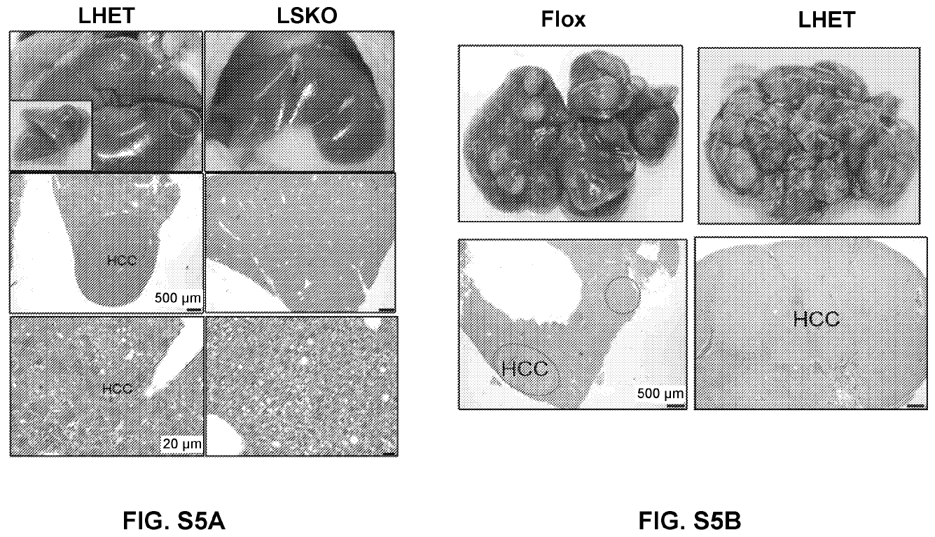

Diethylnitrosamine (DEN) was used to induce HCC. The evaluation was conducted on liver tumor development 24 weeks or 40 weeks after DEN injection. Whereas Flox control mice, LHET mice, and LSKO mice all developed tumors in this model, LSKO mice were relatively protected (FIG. 7B, C; FIG. S7B). Small nodules were found in the livers 24 weeks after DEN injection (FIG. 7B), and tumors were found to be developed by 40 weeks (FIG. 7B, C; FIG. S7B). Most of the LSKO mice had lower tumor burden indicated by lower tumor numbers, smaller tumor sizes, and improved liver pathology, when compared with the Flox control mice (FIG. 7B, C). Furthermore, fewer proliferating cells, indicated by Ki67 labeling, were observed in LSKO mouse livers compared with livers of Flox control mice (FIG. 7D). However, no significant differences were observed in caspase-3 labeling, a marker for apoptosis, suggesting that the reduced tumor burden in LSKO mice is not due to the increased apoptosis (FIG. 7E). In agreement to the LSKO HCC protection phenotype, STAT4, an inhibitor of HCC (49, 50), was identified as an activated upstream regulator of the differentially regulated genes in livers of LSKO mice fed a HFD (FIG. 2A).

To validate whether complete loss of SPTBN1 has a protective effect in human HCC, the frequency of SPTBN1 homozygous loss (deep deletion) was analyzed in human HCC. In data from TCGA, only one HCC patient (0.2%) had a homozygous loss of SPTBN1 (1/440); whereas the heterozygous deletion occurred in 7.8% (30/440) of HCC patients (30/440). Evaluation of copy number alterations in SPTBN1 in 33 cancer types within data in TCGA also showed that homozygous loss of SPTBN1 is rare with an overall frequency of 0.1% across 33 cancer types, representing 33,039 cancer patients (FIG. 7F). Thus, the human cancer results indicate that the findings with the LSKO mice are translationally relevant: Loss of SPTBN1 protects against NASH progression and HCC development.

All of the features described herein (including any accompanying claims, abstract and drawings), and/or all of the steps of any method so disclosed, may be combined with any of the above aspects in any combination, except combinations where at least some of such features and/or steps are mutually exclusive.

It is to be understood that the foregoing aspects and exemplifications are not intended to be limiting in any respect to the scope of the disclosure, and that the claims presented herein are intended to encompass all aspects, embodiments, and exemplifications whether or not explicitly presented herein.

All patents, patent applications, and publications cited herein are fully incorporated by reference in their entirety

### REFERENCE

1. Q. M. Anstee, H. L. Reeves, E. Kotsiliti, O. Govaere, M. Heikenwalder, From NASH to HCC: current concepts and future challenges. Nat Rev Gastroenterol Hepatol 16, 411-428 (2019).
2. Z. Liu et al., Global incidence trends in primary liver cancer by age at diagnosis, sex, region, and etiology, 1990-2017. Cancer 126, 2267-2278 (2020).
3. N. Chalasani et al., The diagnosis and management of nonalcoholic fatty liver disease: Practice guidance from the American Association for the Study of Liver Diseases. Hepatology 67, 328-357 (2018).
4. M. Yarchoan et al., Recent Developments and Therapeutic Strategies against Hepatocellular Carcinoma. Cancer Res 79, 4326-4330 (2019).
5. S. L. Friedman, B. A. Neuschwander-Tetri, M. Rinella, A. J. Sanyal, Mechanisms of NAFLD development and therapeutic strategies. Nat Med 24, 908-922 (2018).
6. B. A. Neuschwander-Tetri, Non-alcoholic fatty liver disease. BMC Med 15, 45 (2017).
7. D. Kumar, G. C. Farrell, C. Fung, J. George, Hepatitis C virus genotype 3 is cytopathic to hepatocytes: Reversal of hepatic steatosis after sustained therapeutic response. Hepatology 36, 1266-1272 (2002).
8. J. Y. Kim et al., ER Stress Drives Lipogenesis and Steatohepatitis via Caspase-2 Activation of S1P. Cell 175, 133-145 e115 (2018).
9. P. Zhao et al., An AMPK-caspase-6 axis controls liver damage in nonalcoholic steatohepatitis. Science 367, 652-660 (2020).
10. H. Shimano, R. Sato, SREBP-regulated lipid metabolism: convergent physiology—divergent pathophysiology. Nat Rev Endocrinol 13, 710-730 (2017).
11. R. A. DeBose-Boyd et al., Transport-dependent proteolysis of SREBP: relocation of site-1 protease from Golgi to ER obviates the need for SREBP transport to Golgi. Cell 99, 703-712 (1999).
12. X. Wang et al., Cleavage of sterol regulatory element binding proteins (SREBPs) by CPP32 during apoptosis. EMBO J 15, 1012-1020 (1996).
13. M. Ekstedt et al., Fibrosis stage is the strongest predictor for disease-specific mortality in NAFLD after up to 33 years of follow-up. Hepatology 61, 1547-1554 (2015).
14. A. Wree, L. Broderick, A. Canbay, H. M. Hoffman, A. E. Feldstein, From NAFLD to NASH to cirrhosis-new insights into disease mechanisms. Nat Rev Gastroenterol Hepatol 10, 627-636 (2013).
15. N. Sanderson et al., Hepatic expression of mature transforming growth factor beta 1 in transgenic mice results in multiple tissue lesions. Proc Natl Acad Sci US A 92, 2572-2576 (1995).
16. K. C. Flanders, Smad3 as a mediator of the fibrotic response. Int J Exp Pathol 85, 47-64 (2004).
17. H. Yadav et al., Protection from obesity and diabetes by blockade of TGF-beta/Smad3 signaling. Cell Metab 14, 67-79 (2011).
18. A. Majumdar et al., Hepatic stem cells and transforming growth factor beta in hepatocellular carcinoma. Nat Rev Gastroenterol Hepatol 9, 530-538 (2012).
19. V. Bennett, Spectrin-based membrane skeleton: a multipotential adaptor between plasma membrane and cytoplasm. Physiol Rev 70, 1029-1065 (1990).
20. V. Bennett, D. N. Lorenzo, An Adaptable Spectrin/Ankyrin-Based Mechanism for Long-Range Organization of Plasma Membranes in Vertebrate Tissues. Curr Top Membr 77, 143-184 (2016).
21. A. Viel, D. Branton, Spectrin: on the path from structure to function. Curr Opin Cell Biol 8, 49-55 (1996).
22. Y. Tang et al., Disruption of transforming growth factor-beta signaling in ELF beta-spectrin-deficient mice. Science 299, 574-577 (2003).
23. J. Chen et al., TGF-beta/beta2-spectrin/CTCF-regulated tumor suppression in human stem cell disorder Beckwith-Wiedemann syndrome. J Clin Invest 126, 527-542 (2016).
24. J. Chen et al., Analysis of Genomes and Transcriptomes of Hepatocellular Carcinomas Identifies Mutations and Gene Expression Changes in the Transforming Growth Factor-beta Pathway. Gastroenterology 154, 195-210 (2018).
25. M. A. De Matteis, J. S. Morrow, Spectrin tethers and mesh in the biosynthetic pathway. J Cell Sci 113 (Pt 13), 2331-2343 (2000).
26. V. Bennett, D. N. Lorenzo, Spectrin- and ankyrin-based membrane domains and the evolution of vertebrates. Curr Top Membr 72, 1-37 (2013).
27. H. J. Baek et al., Caspase-3/7-mediated Cleavage of beta2-spectrin is Required for Acetaminophen-induced Liver Damage. Int J Biol Sci 12, 172-183 (2016).
28. A. Korkut et al., A Pan-Cancer Analysis Reveals High-Frequency Genetic Alterations in Mediators of Signaling by the TGF-beta Superfamily. Cell Syst 7, 422-437 e427 (2018).
29. J. Gutierrez-Cuevas et al., Prolonged-release pirfenidone prevents obesity-induced cardiac steatosis and fibrosis in a mouse NASH model. Cardiovasc Drugs Ther, (2020).
30. A. Sandoval-Rodriguez et al., Pirfenidone Is an Agonistic Ligand for PPARalpha and Improves NASH by Activation of SIRT1/LKB1/pAMPK. Hepatol Commun 4, 434-449 (2020).
31. M. P. Valdecantos et al., A novel glucagon-like peptide 1/glucagon receptor dual agonist improves steatohepatitis and liver regeneration in mice. Hepatology 65, 950-968 (2017).
32. M. I. Lefterova et al., PPARgamma and C/EBP factors orchestrate adipocyte biology via adjacent binding on a genome-wide scale. Genes Dev 22, 2941-2952 (2008).
33. G. Serviddio et al., Uncoupling protein-2 (UCP2) induces mitochondrial proton leak and increases susceptibility of non-alcoholic steatohepatitis (NASH) liver to ischaemia-reperfusion injury. Gut 57, 957-965 (2008).
34. A. Nohturfft, R. A. DeBose-Boyd, S. Scheek, J. L. Goldstein, M. S. Brown, Sterols regulate cycling of SREBP cleavage-activating protein (SCAP) between endoplasmic reticulum and Golgi. Proc Natl Acad Sci USA 96, 11235-11240 (1999).
35. R. Bertolio et al., Sterol regulatory element binding protein 1 couples mechanical cues and lipid metabolism. Nat Commun 10, 1326 (2019).
36. M. S. Brown, J. L. Goldstein, A proteolytic pathway that controls the cholesterol content of membranes, cells, and blood. Proc Natl Acad Sci USA 96, 11041-11048 (1999).
37. B. Knebel et al., Liver-specific expression of transcriptionally active SREBP-1c is associated with fatty liver and increased visceral fat mass. PLoS One 7, e31812 (2012).
38. D. Kozakov et al., The ClusPro web server for protein-protein docking. Nature protocols 12, 255 (2017).
39. D. Kozakov, R. Brenke, S. R. Comeau, S. Vajda, PIPER: an FFT-based protein docking program with pairwise potentials. Proteins: Structure, Function, and Bioinformatics 65, 392-406 (2006).
40. M. Ricchi et al., Differential effect of oleic and palmitic acid on lipid accumulation and apoptosis in cultured hepatocytes. J Gastroenterol Hepatol 24, 830-840 (2009).
41. T. Urbanik et al., Down-regulation of CYLD as a trigger for NF-kappaB activation and a mechanism of apoptotic resistance in hepatocellular carcinoma cells. Int J Oncol 38, 121-131 (2011).
42. K. Tomita et al., Tumour necrosis factor alpha signalling through activation of Kupffer cells plays an essential role in liver fibrosis of non-alcoholic steatohepatitis in mice. Gut 55, 415-424 (2006).
43. F. Wandrer et al., TNF-Receptor-1 inhibition reduces liver steatosis, hepatocellular injury and fibrosis in NAFLD mice. Cell Death Dis 11, 212 (2020).
44. L. Calzadilla Bertot, L. A. Adams, The Natural Course of Non-Alcoholic Fatty Liver Disease. Int J Mol Sci 17, (2016).
45. S. Singh et al., Fibrosis progression in nonalcoholic fatty liver vs nonalcoholic steatohepatitis: a systematic review and meta-analysis of paired-biopsy studies. Clin Gastroenterol Hepatol 13, 643-654 e641-649; quiz e639-640 (2015).
46. B. Hu et al., Therapeutic siRNA: state of the art. Signal Transduct Target Ther 5, 101 (2020).
47. T. Kostrzewski et al., Three-dimensional perfused human in vitro model of non-alcoholic fatty liver disease. World J Gastroenterol 23, 204-215 (2017).
48. T. Kostrzewski et al., A Microphysiological System for Studying Nonalcoholic Steatohepatitis. Hepatol Commun 4, 77-91 (2020).
49. D. K. Jiang et al., Genetic variants in STAT4 and HLA-DQ genes confer risk of hepatitis B virus-related hepatocellular carcinoma. Nat Genet 45, 72-75 (2013).
50. J. Li et al., Clinicopathological significance of STAT4 in hepatocellular carcinoma and its effect on cell growth and apoptosis. Onco Targets Ther 9, 1721-1734 (2016).
51. A. M. Larson et al., Acetaminophen-induced acute liver failure: results of a United States multicenter, prospective study. Hepatology 42, 1364-1372 (2005).
52. T. Yang et al., Crucial step in cholesterol homeostasis: sterols promote binding of SCAP to INSIG-1, a membrane protein that facilitates retention of SREBPs in ER. Cell 110, 489-500 (2002).
53. L. A. Kelley et al., The Phyre2 web portal for protein modeling, prediction and analysis. Nat Protoc 10(6), 845-858 (2015).
54. A. Fiser and A. Sali, ModLoop: automated modeling of loops in protein structures. Bioinformatics 19(18), 2500-2501 (2003).
55. N. Guex and M. C. Peitsch, SWISS-MODEL and the Swiss-PdbViewer: an environment for comparative protein modeling. Electrophoresis 18(15), 2714-2723 (1997).

1. A method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1.

2. The method of claim 1, wherein at least one of the siRNA molecules that inhibits expression of SPTBN1 comprises 15 to 30 nucleotides.

3. The method of claim 2, wherein at least one of the siRNA molecules that inhibits expression of SPTBN1 comprises 15 to 20 nucleotides.

4. The method of claim 1, wherein at least one of the siRNA molecules that inhibits expression of SPTBN1 comprises an overhang region of 1 to 6 nucleotides.

5. The method of claim 1, wherein at least one of the siRNA molecules that inhibits expression of SPTBN1 comprises no overhang region.

6. The method of claim 1, wherein at least one of the siRNA molecules that inhibits expression of SPTBN1 is set forth as SEQ ID No. 7, SEQ ID No. 8, SEQ ID No. 9, or SEQ ID No. 10.

7. A method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 is homologous with at least 10 nucleotides of SEQ ID No. 7, SEQ ID No. 8, SEQ ID No. 9, or SEQ ID No. 10.

8. A method of treating obesity, nonalcohol-related fatty liver disease, nonalcoholic steatohepatitis, or hepatocellular cancer in a subject in need thereof, the method comprising administering a therapeutically effective amount of at least one siRNA molecule that inhibits expression of SPTBN1, wherein at least one of the siRNA molecules that inhibits expression of SPTBN1 has at least 90% sequence identity with SEQ ID No. 7, SEQ ID No. 8, SEQ ID No. 9, or SEQ ID No. 10.

9. The method of claim 1 for treating obesity in a subject in need thereof.

10. The method of claim 9, wherein treating obesity comprises reducing the amount of body fat in the subject and/or reducing the body weight of the subject.

11. The method of claim 1 for treating nonalcohol-related fatty liver disease in a subject in need thereof.

12. The method of claim 11, wherein treating nonalcohol-related fatty liver disease comprises reducing blood triglycerides in the subject.

13. The method of claim 1 for treating nonalcoholic steatohepatitis in a subject in need thereof.

14. The method of claim 13, wherein treating nonalcoholic steatohepatitis comprises reducing blood triglycerides in the subject.

15. The method of claim 1 for treating hepatocellular cancer in a subject in need thereof.

16. The method of claim 15, wherein treating hepatocellular cancer comprises reducing tumor mass in the subject.

17. The method of claim 1, comprising administering one siRNA molecule that inhibits expression of SPTBN1 to the subject.

18. The method of claim 1, comprising administering two siRNA molecules that inhibit expression of SPTBN1 to the subject.

19. The method of claim 1, comprising administering three siRNA molecules that inhibits expression of SPTBN1 to the subject.

20. The method of claim 1, comprising administering four to ten siRNA molecules that inhibits expression of SPTBN1 to the subject.
